(19) United States
(12) Patent Application Publication
(10) Pub. No.: US 2002/0150922 A1
(43) Pub. Date: Oct. 17, 2002

(54) Compositions and methods for the therapy and diagnosis of colon cancer

(75) Inventors: John A. Stolk, Bothell, WA (US); Jiangchun Xu, Bellevue, WA (US); Ruth A. Chenault, Seattle, WA (US); Madeleine Joy Meagher, Seattle, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Corixa Corporation, Seattle, WA (US)

(21) Appl. No.: 09/998,598

(22) Filed: Nov. 16, 2001

Related U.S. Application Data
(60) Provisional application No. 60/304,037, filed on Jul. 10, 2001.
(60) Provisional application No. 60/279,670, filed on Mar. 28, 2001.
(60) Provisional application No. 60/267,011, filed on Feb. 6, 2001.
(60) Provisional application No. 60/252,222, filed on Nov. 20, 2000.

(51) Int. Cl. C12Q 1/68; G01N 33/574; C07H 21/04; C12N 9/00; C12P 21/02; C12N 5/06

(52) U.S. Cl. 435/6; 435/7.23; 435/69.1; 435/183; 435/320.1; 435/325; 536/23.2

(57) ABSTRACT

Compositions and methods for the therapy and diagnosis of cancer, particularly colon cancer, are disclosed. Illustrative compositions comprise one or more colon tumor polypeptides, immunogenic portions thereof, polynucleotides that encode such polypeptides, antigen presenting cell that expresses such polypeptides, and T cells that are specific for cells expressing such polypeptides. The disclosed compositions are useful, for example, in the diagnosis, prevention and/or treatment of diseases, particularly colon cancer.

### BACKGROUND OF THE INVENTION

1. Technical Field of the Invention

The present invention relates generally to therapy and diagnosis of cancer, such as colon cancer. The invention is more specifically related to polypeptides, comprising at least a portion of a colon tumor protein, and to polynucleotides encoding such polypeptides. Such polypeptides and polynucleotides are useful in pharmaceutical compositions, e.g., vaccines, and other compositions for the diagnosis and treatment of colon cancer.

2. Description of the Related Art

Cancer is a significant health problem throughout the world. Although advances have been made in detection and therapy of cancer, no vaccine or other universally successful method for prevention and/or treatment is currently available. Current therapies, which are generally based on a combination of chemotherapy or surgery and radiation, continue to prove inadequate in many patients.

Colon cancer is the second most frequently diagnosed malignancy in the United States as well as the second most common cause of cancer death. The five-year survival rate for patients with colorectal cancer detected in an early localized stage is 92%; unfortunately, only 37% of colorectal cancer is diagnosed at this stage. The survival rate drops to 64% if the cancer is allowed to spread to adjacent organs or lymph nodes, and to 7% in patients with distant metastases.

The prognosis of colon cancer is directly related to the degree of penetration of the tumor through the bowel wall and the presence or absence of nodal involvement, consequently early detection and treatment are especially important. Currently, diagnosis is aided by the use of screening assays for fecal occult blood, sigmoidoscopy, colonoscopy and double contrast barium enemas. Treatment regimens are determined by the type and stage of the cancer, and include surgery, radiation therapy and/or chemotherapy. Recurrence following surgery (the most common form of therapy) is a major problem and is often the ultimate cause of death.

In spite of considerable research into therapies for these and other cancers, colon cancer remains difficult to diagnose and treat effectively. Accordingly, there is a need in the art for improved methods for detecting and treating such cancers. The present invention fulfills these needs and further provides other related advantages.

### BRIEF SUMMARY OF THE INVENTION

In one aspect, the present invention provides polynucleotide compositions comprising a sequence selected from the group consisting of:

(a) sequences provided in SEQ ID NO:1-2589, 2594-2602, and 2604-2605;

(b) complements of the sequences provided in SEQ ID NO:1-2589, 2594-2602, and 2604-2605

(c) sequences consisting of at least 20 contiguous residues of a sequence provided in SEQ ID NO:1-2589, 2594-2602, and 2604-2605;

(d) sequences that hybridize to a sequence provided in SEQ ID NO:1-2589, 2594-2602, and 2604-2605, under moderately stringent conditions;

(e) sequences having at least 75% identity to a sequence of SEQ ID NO:1-2589, 2594-2602, and 2604-2605;

(f) sequences having at least 90% identity to a sequence of SEQ ID NO:1-2589, 2594-2602, and 2604-2605; and

(g) degenerate variants of a sequence provided in SEQ ID NO:1-2589, 2594-2602, and 2604-2605.

In one preferred embodiment, the polynucleotide compositions of the invention are expressed in at least about 20%, more preferably in at least about 30%, and most preferably in at least about 50% of colon tumors samples tested, at a level that is at least about 2-fold, preferably at least about 5-fold, and most preferably at least about 10-fold higher than that for normal tissues.

The present invention, in another aspect, provides polypeptide compositions comprising an amino acid sequence that is encoded by a polynucleotide sequence described above.

The present invention further provides polypeptide compositions comprising an amino acid sequence selected from the group consisting of sequences recited in SEQ ID NOs:2590-2593, 2603, and 2606.

In certain preferred embodiments, the polypeptides and/or polynucleotides of the present invention are immunogenic, i.e., they are capable of eliciting an immune response, particularly a humoral and/or cellular immune response, as further described herein.

The present invention further provides fragments, variants and/or derivatives of the disclosed polypeptide and/or polynucleotide sequences, wherein the fragments, variants and/or derivatives preferably have a level of immunogenic activity of at least about 50%, preferably at least about 70% and more preferably at least about 90% of the level of immunogenic activity of a polypeptide sequence set forth in SEQ ID NOs:2590-2593, 2603, and 2606 or a polypeptide sequence encoded by a polynucleotide sequence set forth in SEQ ID NO:1-2589, 2594-2602, and 2604-2605.

The present invention further provides polynucleotides that encode a polypeptide described above, expression vectors comprising such polynucleotides and host cells transformed or transfected with such expression vectors.

Within other aspects, the present invention provides pharmaceutical compositions comprising a polypeptide or polynucleotide as described above and a physiologically acceptable carrier.

Within a related aspect of the present invention, the pharmaceutical compositions, e.g., vaccine compositions, are provided for prophylactic or therapeutic applications. Such compositions generally comprise an immunogenic polypeptide or polynucleotide of the invention and an immunostimulant, such as an adjuvant.

The present invention further provides pharmaceutical compositions that comprise: (a) an antibody or antigen-binding fragment thereof that specifically binds to a polypeptide of the present invention, or a fragment thereof; and (b) a physiologically acceptable carrier.

Within further aspects, the present invention provides pharmaceutical compositions comprising: (a) an antigen presenting cell that expresses a polypeptide as described above and (b) a pharmaceutically acceptable carrier or excipient. Illustrative antigen presenting cells include dendritic cells, macrophages, monocytes, fibroblasts and B cells.

Within related aspects, pharmaceutical compositions are provided that comprise: (a) an antigen presenting cell that expresses a polypeptide as described above and (b) an immunostimulant.

The present invention further provides, in other aspects, fusion proteins that comprise at least one polypeptide as described above, as well as polynucleotides encoding such fusion proteins, typically in the form of pharmaceutical compositions, e.g., vaccine compositions, comprising a physiologically acceptable carrier and/or an immunostimulant. The fusions proteins may comprise multiple immunogenic polypeptides or portions/variants thereof, as described herein, and may further comprise one or more polypeptide segments for facilitating the expression, purification and/or immunogenicity of the polypeptide(s).

Within further aspects, the present invention provides methods for stimulating an immune response in a patient, preferably a T cell response in a human patient, comprising administering a pharmaceutical composition described herein. The patient may be afflicted with colon cancer, in which case the methods provide treatment for the disease, or patient considered at risk for such a disease may be treated prophylactically.

Within further aspects, the present invention provides methods for inhibiting the development of a cancer in a patient, comprising administering to a patient a pharmaceutical composition as recited above. The patient may be afflicted with colon cancer, in which case the methods provide treatment for the disease, or patient considered at risk for such a disease may be treated prophylactically.

The present invention further provides, within other aspects, methods for removing tumor cells from a biological sample, comprising contacting a biological sample with T cells that specifically react with a polypeptide of the present invention, wherein the step of contacting is performed under conditions and for a time sufficient to permit the removal of cells expressing the protein from the sample.

Within related aspects, methods are provided for inhibiting the development of a cancer in a patient, comprising administering to a patient a biological sample treated as described above.

Methods are further provided, within other aspects, for stimulating and/or expanding T cells specific for a polypeptide of the present invention, comprising contacting T cells with one or more of: (i) a polypeptide as described above; (ii) a polynucleotide encoding such a polypeptide; and/or (iii) an antigen presenting cell that expresses such a polypeptide; under conditions and for a time sufficient to permit the stimulation and/or expansion of T cells. Isolated T cell populations comprising T cells prepared as described above are also provided.

Within further aspects, the present invention provides methods for inhibiting the development of a cancer in a patient, comprising administering to a patient an effective amount of a T cell population as described above.

The present invention further provides methods for inhibiting the development of a cancer in a patient, comprising the steps of: (a) incubating CD4+ and/or CD8+ T cells isolated from a patient with one or more of: (i) a polypeptide comprising at least an immunogenic portion of polypeptide disclosed herein; (ii) a polynucleotide encoding such a polypeptide; and (iii) an antigen-presenting cell that expressed such a polypeptide; and (b) administering to the patient an effective amount of the proliferated T cells, and thereby inhibiting the development of a cancer in the patient. Proliferated cells may, but need not, be cloned prior to administration to the patient.

Within further aspects, the present invention provides methods for determining the presence or absence of a cancer, preferably a colon cancer, in a patient comprising: (a) contacting a biological sample obtained from a patient with a binding agent that binds to a polypeptide as recited above; (b) detecting in the sample an amount of polypeptide that binds to the binding agent; and (c) comparing the amount of polypeptide with a predetermined cut-off value, and therefrom determining the presence or absence of a cancer in the patient. Within preferred embodiments, the binding agent is an antibody, more preferably a monoclonal antibody.

The present invention also provides, within other aspects, methods for monitoring the progression of a cancer in a patient. Such methods comprise the steps of: (a) contacting a biological sample obtained from a patient at a first point in time with a binding agent that binds to a polypeptide as recited above; (b) detecting in the sample an amount of polypeptide that binds to the binding agent; (c) repeating steps (a) and (b) using a biological sample obtained from the patient at a subsequent point in time; and (d) comparing the amount of polypeptide detected in step (c) with the amount detected in step (b) and therefrom monitoring the progression of the cancer in the patient.

The present invention further provides, within other aspects, methods for determining the presence or absence of a cancer in a patient, comprising the steps of: (a) contacting a biological sample obtained from a patient with an oligonucleotide that hybridizes to a polynucleotide that encodes a polypeptide of the present invention; (b) detecting in the sample a level of a polynucleotide, preferably mRNA, that hybridizes to the oligonucleotide; and (c) comparing the level of polynucleotide that hybridizes to the oligonucleotide with a predetermined cut-off value, and therefrom determining the presence or absence of a cancer in the patient. Within certain embodiments, the amount of mRNA is detected via polymerase chain reaction using, for example, at least one oligonucleotide primer that hybridizes to a polynucleotide encoding a polypeptide as recited above, or a complement of such a polynucleotide. Within other embodiments, the amount of mRNA is detected using a hybridization technique, employing an oligonucleotide probe that hybridizes to a polynucleotide that encodes a polypeptide as recited above, or a complement of such a polynucleotide.

In related aspects, methods are provided for monitoring the progression of a cancer in a patient, comprising the steps of: (a) contacting a biological sample obtained from a patient with an oligonucleotide that hybridizes to a polynucleotide that encodes a polypeptide of the present invention; (b) detecting in the sample an amount of a polynucleotide that hybridizes to the oligonucleotide; (c) repeating steps (a) and (b) using a biological sample obtained from the patient at a subsequent point in time; and (d) comparing the amount of polynucleotide detected in step (c) with the amount detected in step (b) and therefrom monitoring the progression of the cancer in the patient.

Within further aspects, the present invention provides antibodies, such as monoclonal antibodies, that bind to a polypeptide as described above, as well as diagnostic kits comprising such antibodies. Diagnostic kits comprising one or more oligonucleotide probes or primers as described above are also provided.

These and other aspects of the present invention will become apparent upon reference to the following detailed description. All references disclosed herein are hereby incorporated by reference in their entirety as if each was incorporated individually.

### BRIEF DESCRIPTION OF THE SEQUENCE IDENTIFIERS

SEQ ID NO:1 is the determined cDNA sequence for clone 62206_contig

SEQ ID NO:2 is the determined cDNA sequence for clone 62207.1

SEQ ID NO:3 is the determined cDNA sequence for clone 62209.2

SEQ ID NO:4 is the determined cDNA sequence for clone 62212.1

SEQ ID NO:5 is the determined cDNA sequence for clone 62213.1

SEQ ID NO:6 is the determined cDNA sequence for clone 62215.2

SEQ ID NO:7 is the determined cDNA sequence for clone 62216.1

SEQ ID NO:8 is the determined cDNA sequence for clone 62242.1

SEQ ID NO:9 is the determined cDNA sequence for clone 62250.2

SEQ ID NO:10 is the determined cDNA sequence for clone 62253_contig

SEQ ID NO:11 is the determined cDNA sequence for clone 62254.2

SEQ ID NO:12 is the determined cDNA sequence for clone 62255.1

SEQ ID NO:13 is the determined cDNA sequence for clone 62256.1

SEQ ID NO:14 is the determined cDNA sequence for clone 62259.1

SEQ ID NO:15 is the determined cDNA sequence for clone 62261_contig

SEQ ID NO:16 is the determined cDNA sequence for clone 62269.2

SEQ ID NO:17 is the determined cDNA sequence for clone 62275.1

SEQ ID NO:18 is the determined cDNA sequence for clone 62277.1

SEQ ID NO:19 is the determined cDNA sequence for clone 62279_contig

SEQ ID NO:20 is the determined cDNA sequence for clone 6228 1.1

SEQ ID NO:21 is the determined cDNA sequence for clone 62284.2

SEQ ID NO:22 is the determined cDNA sequence for clone 62285.2

SEQ ID NO:23 is the determined cDNA sequence for clone 62286.1

SEQ ID NO:24 is the determined cDNA sequence for clone 62287.1

SEQ ID NO:25 is the determined cDNA sequence for clone 62288.2

SEQ ID NO:26 is the determined cDNA sequence for clone 62290_contig

SEQ ID NO:27 is the determined cDNA sequence for clone 62292.2

SEQ ID NO:28 is the determined cDNA sequence for clone 62293.1

SEQ ID NO:29 is the determined cDNA sequence for clone 62294.1

SEQ ID NO:30 is the determined cDNA sequence for clone 62295.1

SEQ ID NO:31 is the determined cDNA sequence for clone 62296.1

SEQ ID NO:32 is the determined cDNA sequence for clone 62297.1

SEQ ID NO:33 is the determined cDNA sequence for clone 62298_contig

SEQ ID NO:34 is the determined cDNA sequence for clone 62301.2

SEQ ID NO:35 is the determined cDNA sequence for clone 62310.2

SEQ ID NO:36 is the determined cDNA sequence for clone 62313.2

SEQ ID NO:37 is the determined cDNA sequence for clone 62315.1

SEQ ID NO:38 is the determined cDNA sequence for clone 62316.2

SEQ ID NO:39 is the determined cDNA sequence for clone 62318.1

SEQ ID NO:40 is the determined cDNA sequence for clone 62323.2

SEQ ID NO:41 is the determined cDNA sequence for clone 62324.2

SEQ ID NO:42 is the determined cDNA sequence for clone 62329.1

SEQ ID NO:43 is the determined cDNA sequence for clone 62330.1

SEQ ID NO:44 is the determined cDNA sequence for clone 62333.1

SEQ ID NO:45 is the determined cDNA sequence for clone 63644_contig

SEQ ID NO:46 is the determined cDNA sequence for clone 63649.2

SEQ ID NO:47 is the determined cDNA sequence for clone 63650.1

SEQ ID NO:48 is the determined cDNA sequence for clone 63652.2

SEQ ID NO:49 is the determined cDNA sequence for clone 63658

SEQ ID NO:50 is the determined cDNA sequence for clone 63659.1

SEQ ID NO:51 is the determined cDNA sequence for clone 63661.1

SEQ ID NO:52 is the determined cDNA sequence for clone 63663.2

SEQ ID NO:53 is the determined cDNA sequence for clone 63666.1

SEQ ID NO:54 is the determined cDNA sequence for clone 63667.1

SEQ ID NO:55 is the determined cDNA sequence for clone 63668.2

SEQ ID NO:56 is the determined cDNA sequence for clone 63670.1

SEQ ID NO:57 is the determined cDNA sequence for clone 63672.1

SEQ ID NO:58 is the determined cDNA sequence for clone 63673.2

SEQ ID NO:59 is the determined cDNA sequence for clone 63675.2

SEQ ID NO:60 is the determined cDNA sequence for clone 63676.1

SEQ ID NO:61 is the determined cDNA sequence for clone 63677.1

SEQ ID NO:62 is the determined cDNA sequence for clone 63682.1

SEQ ID NO:63 is the determined cDNA sequence for clone 63683.2

SEQ ID NO:64 is the determined cDNA sequence for clone 63688.2

SEQ ID NO:65 is the determined cDNA sequence for clone 63693.2

SEQ ID NO:66 is the determined cDNA sequence for clone 63696.1

SEQ ID NO:67 is the determined cDNA sequence for clone 63697.2

SEQ ID NO:68 is the determined cDNA sequence for clone 63698.1

SEQ ID NO:69 is the determined cDNA sequence for clone 63949.2

SEQ ID NO:70 is the determined cDNA sequence for clone 63950.2

SEQ ID NO:71 is the determined cDNA sequence for clone 63956.2

SEQ ID NO:72 is the determined cDNA sequence for clone 62410.1

SEQ ID NO:73 is the determined cDNA sequence for clone 62412.1

SEQ ID NO:74 is the determined cDNA sequence for clone 62413.1

SEQ ID NO:75 is the determined cDNA sequence for clone 62414.1

SEQ ID NO:76 is the determined cDNA sequence for clone 62415.1

SEQ ID NO:77 is the determined cDNA sequence for clone 62416.1

SEQ ID NO:78 is the determined cDNA sequence for clone 62417.1

SEQ ID NO:79 is the determined cDNA sequence for clone 62418.1

SEQ ID NO:80 is the determined cDNA sequence for clone 62419.1

SEQ ID NO:81 is the determined cDNA sequence for clone 62420.1

SEQ ID NO:82 is the determined cDNA sequence for clone 6242 1.1

SEQ ID NO:83 is the determined cDNA sequence for clone 62422.1

SEQ ID NO:84 is the determined cDNA sequence for clone 62423.1

SEQ ID NO:85 is the determined cDNA sequence for clone 62424.1

SEQ ID NO:86 is the determined cDNA sequence for clone 62425.1

SEQ ID NO:87 is the determined cDNA sequence for clone 62426.1

SEQ ID NO:88 is the determined cDNA sequence for clone 62427.1

SEQ ID NO:89 is the determined cDNA sequence for clone 62429.1

SEQ ID NO:90 is the determined cDNA sequence for clone 62430.1

SEQ ID NO:91 is the determined cDNA sequence for clone 62431.1

SEQ ID NO:92 is the determined cDNA sequence for clone 62432.1

SEQ ID NO:93 is the determined cDNA sequence for clone 62433.1

SEQ ID NO:94 is the determined cDNA sequence for clone 62434.1

SEQ ID NO:95 is the determined cDNA sequence for clone 62435.1

SEQ ID NO:96 is the determined cDNA sequence for clone 62436.1

SEQ ID NO:97 is the determined cDNA sequence for clone 62437.1

SEQ ID NO:98 is the determined cDNA sequence for clone 62438.1

SEQ ID NO:99 is the determined cDNA sequence for clone 62439.1

SEQ ID NO:100 is the determined cDNA sequence for clone 62440.1

SEQ ID NO:101 is the determined cDNA sequence for clone 62441.1

SEQ ID NO:102 is the determined cDNA sequence for clone 62442.1

SEQ ID NO:103 is the determined cDNA sequence for clone 62443.1

SEQ ID NO:104 is the determined cDNA sequence for clone 62444.1

SEQ ID NO:105 is the determined cDNA sequence for clone 62445.1

SEQ ID NO:106 is the determined cDNA sequence for clone 62446.1

SEQ ID NO:107 is the determined cDNA sequence for clone 62448.1

SEQ ID NO:108 is the determined cDNA sequence for clone 62449.1

SEQ ID NO:109 is the determined cDNA sequence for clone 62450.1

SEQ ID NO:110 is the determined cDNA sequence for clone 62451.1

SEQ ID NO:111 is the determined cDNA sequence for clone 62452.1

SEQ ID NO:112 is the determined cDNA sequence for clone 62453.1

SEQ ID NO:113 is the determined cDNA sequence for clone 62454.1

SEQ ID NO:114 is the determined cDNA sequence for clone 62455.1

SEQ ID NO:115 is the determined cDNA sequence for clone 62456.1

SEQ ID NO:116 is the determined cDNA sequence for clone 62457.1

SEQ ID NO:117 is the determined cDNA sequence for clone 62460.1

SEQ ID NO:118 is the determined cDNA sequence for clone 62461.1

SEQ ID NO:119 is the determined cDNA sequence for clone 62462.1

SEQ ID NO:120 is the determined cDNA sequence for clone 62464.1

SEQ ID NO:121 is the determined cDNA sequence for clone 62465.1

SEQ ID NO:122 is the determined cDNA sequence for clone 62467.1

SEQ ID NO:123 is the determined cDNA sequence for clone 62468.1

SEQ ID NO:124 is the determined cDNA sequence for clone 62469.1

SEQ ID NO:125 is the determined cDNA sequence for clone 62470.1

SEQ ID NO:126 is the determined cDNA sequence for clone 62471.1

SEQ ID NO:127 is the determined cDNA sequence for clone 62472.1

SEQ ID NO:128 is the determined cDNA sequence for clone 62473.1

SEQ ID NO:129 is the determined cDNA sequence for clone 62474.1

SEQ ID NO:130 is the determined cDNA sequence for clone 62475.1

SEQ ID NO:131 is the determined cDNA sequence for clone 62476.1

SEQ ID NO:132 is the determined cDNA sequence for clone 62477.1

SEQ ID NO:133 is the determined cDNA sequence for clone 62478.1

SEQ ID NO:134 is the determined cDNA sequence for clone 62479.1

SEQ ID NO:135 is the determined cDNA sequence for clone 62480.1

SEQ ID NO:136 is the determined cDNA sequence for clone 62481.1

SEQ ID NO:137 is the determined cDNA sequence for clone 62482.1

SEQ ID NO:138 is the determined cDNA sequence for clone 62483.1

SEQ ID NO:139 is the determined cDNA sequence for clone 62484.1

SEQ ID NO:140 is the determined cDNA sequence for clone 62485.1

SEQ ID NO:141 is the determined cDNA sequence for clone 62486.1

SEQ ID NO:142 is the determined cDNA sequence for clone 62487.1

SEQ ID NO:143 is the determined cDNA sequence for clone 62488.1

SEQ ID NO:144 is the determined cDNA sequence for clone 62489.1

SEQ ID NO:145 is the determined cDNA sequence for clone 62490.1

SEQ ID NO:146 is the determined cDNA sequence for clone 62491.1

SEQ ID NO:147 is the determined cDNA sequence for clone 62492.1

SEQ ID NO:148 is the determined cDNA sequence for clone 62493.1

SEQ ID NO:149 is the determined cDNA sequence for clone 62494.1

SEQ ID NO:150 is the determined cDNA sequence for clone 62495.1

SEQ ID NO:151 is the determined cDNA sequence for clone 62496.1

SEQ ID NO:152 is the determined cDNA sequence for clone 62497.1

SEQ ID NO:153 is the determined cDNA sequence for clone 62498.1

SEQ ID NO:154 is the determined cDNA sequence for clone 62499.1

SEQ ID NO:155is the determined cDNA sequence for clone Contig_{—}133_{—}62500

SEQ ID NO:156 is the determined cDNA sequence for clone 62501

SEQ ID NO:157 is the determined cDNA sequence for clone 62502.1

SEQ ID NO:158 is the determined cDNA sequence for clone 62503.1

SEQ ID NO:159 is the determined cDNA sequence for clone 62504.1

SEQ ID NO:160 is the determined cDNA sequence for clone 62505.1

SEQ ID NO:161 is the determined cDNA sequence for clone 62506.1

SEQ ID NO:162 is the determined cDNA sequence for clone 62507.1

SEQ ID NO:163 is the determined cDNA sequence for clone 62508.1

SEQ ID NO:164 is the determined cDNA sequence for clone 62509.1

SEQ ID NO:165 is the determined cDNA sequence for clone 62510.1

SEQ ID NO:166 is the determined cDNA sequence for clone 62511.1

SEQ ID NO:167 is the determined cDNA sequence for clone 62512.1

SEQ ID NO:168 is the determined cDNA sequence for clone 62513.1

SEQ ID NO:169 is the determined cDNA sequence for clone 62514.1

SEQ ID NO:170 is the determined cDNA sequence for clone 62515.1

SEQ ID NO:171 is the determined cDNA sequence for clone 62516.1

SEQ ID NO:172 is the determined cDNA sequence for clone 62517

SEQ ID NO:173 is the determined cDNA sequence for clone 62518.1

SEQ ID NO:174 is the determined cDNA sequence for clone 62519.1

SEQ ID NO:175 is the determined cDNA sequence for clone 62520.1

SEQ ID NO:176 is the determined cDNA sequence for clone 62521.1

SEQ ID NO:177 is the determined cDNA sequence for clone 62522.1

SEQ ID NO:178 is the determined cDNA sequence for clone 62524.1

SEQ ID NO:179 is the determined cDNA sequence for clone 62525.1

SEQ ID NO:180 is the determined cDNA sequence for clone Contig_{—}146_{—}62526

SEQ ID NO:181 is the determined cDNA sequence for clone 62527.1

SEQ ID NO:182 is the determined cDNA sequence for clone 62528.1

SEQ ID NO:183 is the determined cDNA sequence for clone 62529.1

SEQ ID NO:184 is the determined cDNA sequence for clone 62530.1

SEQ ID NO:185 is the determined cDNA sequence for clone 62531.1

SEQ ID NO:186 is the determined cDNA sequence for clone 62532.1

SEQ ID NO:187 is the determined cDNA sequence for clone 62533.1

SEQ ID NO:188 is the determined cDNA sequence for clone 62534.1

SEQ ID NO:189 is the determined cDNA sequence for clone 62535.1

SEQ ID NO:190is the determined cDNA sequence for clone Contig_{—}213_{—}62536

SEQ ID NO:191 is the determined cDNA sequence for clone 62537

SEQ ID NO:192 is the determined cDNA sequence for clone 62538.1

SEQ ID NO:193 is the determined cDNA sequence for clone 62539.1

SEQ ID NO:1 94 is the determined cDNA sequence for clone 62540.1

SEQ ID NO:195 is the determined cDNA sequence for clone 62541.1

SEQ ID NO:196 is the determined cDNA sequence for clone 62542.1

SEQ ID NO:197 is the determined cDNA sequence for clone 62543.1

SEQ ID NO:198 is the determined cDNA sequence for clone Contig_{—}62_{—}62544

SEQ ID NO:199 is the determined cDNA sequence for clone 62545.1

SEQ ID NO:200 is the determined cDNA sequence for clone 62547.1

SEQ ID NO:201 is the determined cDNA sequence for clone 62548.1

SEQ ID NO:202 is the determined cDNA sequence for clone 62549.1

SEQ ID NO:203 is the determined cDNA sequence for clone 62550.1

SEQ ID NO:204 is the determined cDNA sequence for clone 62551.1

SEQ ID NO:205 is the determined cDNA sequence for clone 62553.1

SEQ ID NO:206 is the determined cDNA sequence for clone 62554.1

SEQ ID NO:207 is the determined cDNA sequence for clone 62555.1

SEQ ID NO:208 is the determined cDNA sequence for clone 62556.1

SEQ ID NO:209 is the determined cDNA sequence for clone 62557.1

SEQ ID NO:210 is the determined cDNA sequence for clone 62558.1

SEQ ID NO:211 is the determined cDNA sequence for clone 62559.1

SEQ ID NO:212 is the determined cDNA sequence for clone 62560.1

SEQ ID NO:213 is the determined cDNA sequence for clone 62561.1

SEQ ID NO:214 is the determined cDNA sequence for clone 62562.1

SEQ ID NO:215 is the determined cDNA sequence for clone 62563.1

SEQ ID NO:216 is the determined cDNA sequence for clone Contig_{—}193_{—}62564

SEQ ID NO:217 is the determined cDNA sequence for clone 62565.1

SEQ ID NO:218 is the determined cDNA sequence for clone 62566.1

SEQ ID NO:219 is the determined cDNA sequence for clone 62567.1

SEQ ID NO:220 is the determined cDNA sequence for clone 62568.1

SEQ ID NO:221 is the determined cDNA sequence for clone 62569.1

SEQ ID NO:222 is the determined cDNA sequence for clone 62570.1

SEQ ID NO:223 is the determined cDNA sequence for clone 62571.1

SEQ ID NO:224 is the determined cDNA sequence for clone 62572.1

SEQ ID NO:225 is the determined cDNA sequence for clone Contig_{—}198_{—}62573

SEQ ID NO:226 is the determined cDNA sequence for clone 62574.1

SEQ ID NO:227 is the determined cDNA sequence for clone 62575.1

SEQ ID NO:228 is the determined cDNA sequence for clone 62576.1

SEQ ID NO:229 is the determined cDNA sequence for clone 62577.1

SEQ ID NO:230 is the determined cDNA sequence for clone 62578.1

SEQ ID NO:231 is the determined cDNA sequence for clone 62579.1

SEQ ID NO:232 is the determined cDNA sequence for clone 62581.1

SEQ ID NO:233 is the determined cDNA sequence for clone 62582.1

SEQ ID NO:234 is the determined cDNA sequence for clone 62583.1

SEQ ID NO:235 is the determined cDNA sequence for clone 62585.1

SEQ ID NO:236 is the determined cDNA sequence for clone 62587.1

SEQ ID NO:237 is the determined cDNA sequence for clone 62589.1

SEQ ID NO:238 is the determined cDNA sequence for clone 62590.1

SEQ ID NO:239 is the determined cDNA sequence for clone 62591.1

SEQ ID NO:240 is the determined cDNA sequence for clone Contig_{—}207_{—}62592

SEQ ID NO:241 is the determined cDNA sequence for clone Contig_{—}18_{—}62593

SEQ ID NO:242 is the determined cDNA sequence for clone 62594.1

SEQ ID NO:243 is the determined cDNA sequence for clone 62595.1

SEQ ID NO:244 is the determined cDNA sequence for clone 62596.1

SEQ ID NO:245 is the determined cDNA sequence for clone 62597.1

SEQ ID NO:246 is the determined cDNA sequence for clone 62598.1

SEQ ID NO:247 is the determined cDNA sequence for clone 62599.1

SEQ ID NO:248 is the determined cDNA sequence for clone 62600.1

SEQ ID NO:249 is the determined cDNA sequence for clone 62601.1

SEQ ID NO:250 is the determined cDNA sequence for clone 62602.1

SEQ ID NO:251 is the determined cDNA sequence for clone 62603.1

SEQ ID NO:252 is the determined cDNA sequence for clone 62604.1

SEQ ID NO:253 is the determined cDNA sequence for clone 62605.1

SEQ ID NO:254 is the determined cDNA sequence for clone 62606.1

SEQ ID NO:255 is the determined cDNA sequence for clone 62607.1

SEQ ID NO:256 is the determined cDNA sequence for clone 62608.1

SEQ ID NO:257 is the determined cDNA sequence for clone 62609.1

SEQ ID NO:258 is the determined cDNA sequence for clone 62610.1

SEQ ID NO:259 is the determined cDNA sequence for clone 62611.1

SEQ ID NO:260 is the determined cDNA sequence for clone 62613.1

SEQ ID NO:261 is the determined cDNA sequence for clone 62614.1

SEQ ID NO:262 is the determined cDNA sequence for clone 62615.1

SEQ ID NO:263 is the determined cDNA sequence for clone 62616.1

SEQ ID NO:264 is the determined cDNA sequence for clone 62617.1

SEQ ID NO:265 is the determined cDNA sequence for clone 62618.1

SEQ ID NO:266 is the determined cDNA sequence for clone 62620.1

SEQ ID NO:267 is the determined cDNA sequence for clone 62621.1

SEQ ID NO:268 is the determined cDNA sequence for clone 62622.1

SEQ ID NO:269 is the determined cDNA sequence for clone 62623.1

SEQ ID NO:270 is the determined cDNA sequence for clone 62624.1

SEQ ID NO:271 is the determined cDNA sequence for clone 62625.1

SEQ ID NO:272 is the determined cDNA sequence for clone 62626.1

SEQ ID NO:273 is the determined cDNA sequence for clone 62627.1

SEQ ID NO:274 is the determined cDNA sequence for clone 62628.1

SEQ ID NO:275 is the determined cDNA sequence for clone 62629.1

SEQ ID NO:276 is the determined cDNA sequence for clone 62630.1

SEQ ID NO:277 is the determined cDNA sequence for clone 62631.1

SEQ ID NO:278 is the determined cDNA sequence for clone 62632.1

SEQ ID NO:279 is the determined cDNA sequence for clone 62633.1

SEQ ID NO:280 is the determined cDNA sequence for clone 62634.1

SEQ ID NO:281 is the determined cDNA sequence for clone 62635.1

SEQ ID NO:282 is the determined cDNA sequence for clone 62636.1

SEQ ID NO:283 is the determined cDNA sequence for clone 62637.1

SEQ ID NO:284 is the determined cDNA sequence for clone 62638.1

SEQ ID NO:285 is the determined cDNA sequence for clone Contig_{—}131_{—}62639

SEQ ID NO:286 is the determined cDNA sequence for clone 62640.1

SEQ ID NO:287 is the determined cDNA sequence for clone 62641.1

SEQ ID NO:288 is the determined cDNA sequence for clone 62642.1

SEQ ID NO:289 is the determined cDNA sequence for clone 62643.1

SEQ ID NO:290 is the determined cDNA sequence for clone 62644.1

SEQ ID NO:291 is the determined cDNA sequence for clone 62645.1

SEQ ID NO:292 is the determined cDNA sequence for clone 62646.1

SEQ ID NO:293 is the determined cDNA sequence for clone 62647.1

SEQ ID NO:294 is the determined cDNA sequence for clone 62648.1

SEQ ID NO:295 is the determined cDNA sequence for clone 62649.1

SEQ ID NO:296 is the determined cDNA sequence for clone 62650.1

SEQ ID NO:297 is the determined cDNA sequence for clone 62651.1

SEQ ID NO:298 is the determined cDNA sequence for clone 62652.1

SEQ ID NO:299 is the determined cDNA sequence for clone 62653.1

SEQ ID NO:300 is the determined cDNA sequence for clone 62654.1

SEQ ID NO:301 is the determined cDNA sequence for clone 62655.1

SEQ ID NO:302 is the determined cDNA sequence for clone 62656.1

SEQ ID NO:303 is the determined cDNA sequence for clone 62657.1

SEQ ID NO:304 is the determined cDNA sequence for clone 62658.1

SEQ ID NO:305 is the determined cDNA sequence for clone 62659.1

SEQ ID NO:306 is the determined cDNA sequence for clone 62660.1

SEQ ID NO:307 is the determined cDNA sequence for clone 62661.1

SEQ ID NO:308 is the determined cDNA sequence for clone 62662.1

SEQ ID NO:309 is the determined cDNA sequence for clone 62663.1

SEQ ID NO:310 is the determined cDNA sequence for clone 62664.1

SEQ ID NO:311 is the determined cDNA sequence for clone 62665.1

SEQ ID NO:312 is the determined cDNA sequence for clone 62666.1

SEQ ID NO:313 is the determined cDNA sequence for clone 62667.1

SEQ ID NO:314 is the determined cDNA sequence for clone 62668.1

SEQ ID NO:315 is the determined cDNA sequence for clone 62669.1

SEQ ID NO:316 is the determined cDNA sequence for clone 62670.1

SEQ ID NO:317 is the determined cDNA sequence for clone 62671.1

SEQ ID NO:318 is the determined cDNA sequence for clone 62672.1

SEQ ID NO:319 is the determined cDNA sequence for clone 62673.1

SEQ ID NO:320 is the determined cDNA sequence for clone 62674.1

SEQ ID NO:321 is the determined cDNA sequence for clone 62675.1

SEQ ID NO:322 is the determined cDNA sequence for clone 62676.1

SEQ ID NO:323 is the determined cDNA sequence for clone 62677.1

SEQ ID NO:324 is the determined cDNA sequence for clone 62678.1

SEQ ID NO:325 is the determined cDNA sequence for clone 62679.1

SEQ ID NO:326 is the determined cDNA sequence for clone 62680.1

SEQ ID NO:327 is the determined cDNA sequence for clone 62681.1

SEQ ID NO:328 is the determined cDNA sequence for clone 62682.1

SEQ ID NO:329 is the determined cDNA sequence for clone 62683.1

SEQ ID NO:330 is the determined cDNA sequence for clone 62684.1

SEQ ID NO:331 is the determined cDNA sequence for clone 62685.1

SEQ ID NO:332 is the determined cDNA sequence for clone 62686.1

SEQ ID NO:333 is the determined cDNA sequence for clone 62687.1

SEQ ID NO:334 is the determined cDNA sequence for clone 62688.1

SEQ ID NO:335 is the determined cDNA sequence for clone 62689.1

SEQ ID NO:336 is the determined cDNA sequence for clone 62690.1

SEQ ID NO:337 is the determined cDNA sequence for clone 62691.1

SEQ ID NO:338 is the determined cDNA sequence for clone 62692.1

SEQ ID NO:339 is the determined cDNA sequence for clone 62693.1

SEQ ID NO:340 is the determined cDNA sequence for clone 62694.1

SEQ ID NO:341 is the determined cDNA sequence for clone 62695.1

SEQ ID NO:342 is the determined cDNA sequence for clone 62696.1

SEQ ID NO:343 is the determined cDNA sequence for clone 62697.1

SEQ ID NO:344 is the determined cDNA sequence for clone 62722.1

SEQ ID NO:345 is the determined cDNA sequence for clone 62725.1

SEQ ID NO:346 is the determined cDNA sequence for clone 62728.1

SEQ ID NO:347 is the determined cDNA sequence for clone 62729.1

SEQ ID NO:348 is the determined cDNA sequence for clone 62731.1

SEQ ID NO:349 is the determined cDNA sequence for clone 67663.3_{—}

SEQ ID NO:350 is the determined cDNA sequence for clone 67664.3_{—}

SEQ ID NO:351 is the determined cDNA sequence for clone 67665.4_{—}

SEQ ID NO:352 is the determined cDNA sequence for clone C615S_{—}63676

SEQ ID NO:353 is the determined cDNA sequence for clone C616S_{—}63949

SEQ ID NO:354 is the determined cDNA sequence for clone C617S_{—}62290

SEQ ID NO:355 is the determined cDNA sequence for clone C618S_{—}63693

SEQ ID NO:356 is the determined cDNA sequence for clone C619S_{—}62293

SEQ ID NO:357 is the determined cDNA sequence for clone C620S_{—}63677

SEQ ID NO:358 is the determined cDNA sequence for clone C621S_{—}62294

SEQ ID NO:359 is the determined cDNA sequence for clone C622S_{—}63659

SEQ ID NO:360 is the determined cDNA sequence for clone C623S_{—}62301

SEQ ID NO:361 is the determined cDNA sequence for clone C624S_{—}63663

SEQ ID NO:362 is the determined cDNA sequence for clone C625S_{—}62324

SEQ ID NO:363 is the determined cDNA sequence for clone C626S_{—}62213

SEQ ID NO:364 is the determined cDNA sequence for clone

FLJ21409_fis_clone_COL03924.seq_related_to_C618S_{—}63693

SEQ ID NO:365 is the determined cDNA sequence for clone

Human_eukaryotic_initiation_factor_{—}4E.seq_related_to_C624S_{—}63663

SEQ ID NO:366 is the determined cDNA sequence for clone

KIAA0905.seq_related_to_C622S_{—}63659

SEQ ID NO:367 is the determined cDNA sequence for clone

p35_CAK1_associated_protein.seq_related_to_C616S_{—}63949

SEQ ID NO:368 is the determined cDNA sequence for clone

L6_Human_Tumor_Antigen.seq_related_to_C620S_{—}63677

SEQ ID NO:369 is the determined cDNA sequence for clone

ARF_GTPase_activating_protein_GIT1.seq_related_to_C621S_{—}62294

SEQ ID NO:370 is the determined cDNA sequence for clone

KIAA0104.seq_related_to_CC615S_{—}63676

SEQ ID NO:371 is the determined cDNA sequence for clone 62218571 R0374:A05

SEQ ID NO:372 is the determined cDNA sequence for clone 62218572 R0374:A06

SEQ ID NO:373 is the determined cDNA sequence for clone 62218575 R0374:A09

SEQ ID NO:374 is the determined cDNA sequence for clone 62218577 R0374:A11

SEQ ID NO:375 is the determined cDNA sequence for clone 62218578 R0374:A12

SEQ ID NO:376 is the determined cDNA sequence for clone 62218579 R0374:B01

SEQ ID NO:377 is the determined cDNA sequence for clone 62218580 R0374:B02

SEQ ID NO:378 is the deter-mined cDNA sequence for clone 62218582 R0374:B4

SEQ ID NO:379 is the determined cDNA sequence for clone 62218585 R0374:B07

SEQ ID NO:380 is the determined cDNA sequence for clone 62218586 R0374:B08

SEQ ID NO:381 is the determined cDNA sequence for clone 62218587 R0374:B09

SEQ ID NO:382 is the determined cDNA sequence for clone 62218588 R0374:B10

SEQ ID NO:383 is the determined cDNA sequence for clone 62218591 R0374:C01

SEQ ID NO:384 is the determined cDNA sequence for clone 62218592 R0374:C02

SEQ ID NO:385 is the determined cDNA sequence for clone 62218593 R0374:C03

SEQ ID NO:386 is the determined cDNA sequence for clone 62218594 R0374:C04

SEQ ID NO:387 is the determined cDNA sequence for clone 62218595 R0374:C05

SEQ ID NO:388 is the determined cDNA sequence for clone 62218596 R0374:C06

SEQ ID NO:389 is the determined cDNA sequence for clone 62218597 R0374:C07

SEQ ID NO:390 is the determined cDNA sequence for clone 62218598 R0374:C08

SEQ ID NO:391 is the determined cDNA sequence for clone 62218599 R0374:C09

SEQ ID NO:392 is the determined cDNA sequence for clone 62218600 R0374:C10

SEQ ID NO:393 is the determined cDNA sequence for clone 62218601 R0374:C11

SEQ ID NO:394 is the determined cDNA sequence for clone 62218603 R0374:D01

SEQ ID NO:395 is the determined cDNA sequence for clone 62218604 R0374:D02

SEQ ID NO:396 is the determined cDNA sequence for clone 62218605 R0374:D03

SEQ ID NO:397 is the determined cDNA sequence for clone 62218607 R0374:D05

SEQ ID NO:398 is the determined cDNA sequence for clone 62218609 R0374:D07

SEQ ID NO:399 is the determined cDNA sequence for clone 62218611 R0374:D09

SEQ ID NO:400 is the determined cDNA sequence for clone 62218612 R0374:D10

SEQ ID NO:401 is the determined cDNA sequence for clone 62218613 R0374:D11

SEQ ID NO:402 is the determined cDNA sequence for clone 62218614 R0374:D12

SEQ ID NO:403 is the determined cDNA sequence for clone 62218615 R0374:E01

SEQ ID NO:404 is the determined cDNA sequence for clone 62218617 R0374:E03

SEQ ID NO:405 is the determined cDNA sequence for clone 62218618 R0374:E04

SEQ ID NO:406 is the determined cDNA sequence for clone 62218619 R0374:E05

SEQ ID NO:407 is the determined cDNA sequence for clone 62218620 R0374:E06

SEQ ID NO:408 is the determined cDNA sequence for clone 62218621 R0374:E07

SEQ ID NO:409 is the determined cDNA sequence for clone 62218622 R0374:E08

SEQ ID NO:410 is the determined cDNA sequence for clone 62218623 R0374:E09

SEQ ID NO:411 is the determined cDNA sequence for clone 62218624 R0374:E10

SEQ ID NO:412 is the determined cDNA sequence for clone 62218625 R0374:E11

SEQ ID NO:413 is the determined cDNA sequence for clone 62218626 R0374:E12

SEQ ID NO:414 is the determined cDNA sequence for clone 62218627 R0374:F01

SEQ ID NO:415 is the determined cDNA sequence for clone 62218628 R0374:F02

SEQ ID NO:416 is the determined cDNA sequence for clone 62218629 R0374:F03

SEQ ID NO:417 is the determined cDNA sequence for clone 62218631 R0374:F05

SEQ ID NO:418 is the determined cDNA sequence for clone 62218632 R0374:F06

SEQ ID NO:419 is the determined cDNA sequence for clone 62218633 R0374:F07

SEQ ID NO:420 is the determined cDNA sequence for clone 62218634 R0374:F08

SEQ ID NO:421 is the determined cDNA sequence for clone 62218635 R0374:F:09

SEQ ID NO:422 is the determined cDNA sequence for clone 62218636 R0374:F10

SEQ ID NO:423 is the determined cDNA sequence for clone 62218638 R0374:F12

SEQ ID NO:424 is the determined cDNA sequence for clone 62218639 R0374:G01

SEQ ID NO:425 is the determined cDNA sequence for clone 62218640 R0374:G02

SEQ ID NO:426 is the determined cDNA sequence for clone 62218642 R0374:G04

SEQ ID NO:427 is the determined cDNA sequence for clone 62218643 R0374:G05

SEQ ID NO:428 is the determined cDNA sequence for clone 62218644 R0374: G06

SEQ ID NO:429 is the determined cDNA sequence for clone 62218645 R0374:G07

SEQ ID NO:430 is the determined cDNA sequence for clone 62218646 R0374:G08

SEQ ID NO:431 is the determined cDNA sequence for clone 62218647 R0374:G09

SEQ ID NO:432 is the determined cDNA sequence for clone 62218648 R0374:G10

SEQ ID NO:433 is the determined cDNA sequence for clone 62218649 R0374:G11

SEQ ID NO:434 is the determined cDNA sequence for clone 62218652 R0374:H02

SEQ ID NO:435 is the determined cDNA sequence for clone 62218653 R0374:H03

SEQ ID NO:436 is the determined cDNA sequence for clone 62218654 R0374:H04

SEQ ID NO:437 is the determined cDNA sequence for clone 62218655 R0374:H05

SEQ ID NO:438 is the determined cDNA sequence for clone 62218656 R0374:H06

SEQ ID NO:439 is the determined cDNA sequence for clone 62218657 R0374:H07

SEQ ID NO:440 is the determined cDNA sequence for clone 62218658 R0374:H:08

SEQ ID NO:441 is the determined cDNA sequence for clone 62218659 R0374:H09

SEQ ID NO:442 is the determined cDNA sequence for clone 62218660 R0374:H10

SEQ ID NO:443 is the determined cDNA sequence for clone 62218661 R0374:H11

SEQ ID NO:444 is the determined cDNA sequence for clone 62116844 R0375:A02

SEQ ID NO:445 is the determined cDNA sequence for clone 62116845 R0375:A03

SEQ ID NO:446 is the determined cDNA sequence for clone 62116847 R0375:A06

SEQ ID NO:447 is the determined cDNA sequence for clone 62116848 R0375:A07

SEQ ID NO:448 is the determined cDNA sequence for clone 62116849 R0375:A:08

SEQ ID NO:449 is the determined cDNA sequence for clone 62116850 R0375:A09

SEQ ID NO:450 is the determined cDNA sequence for clone 62116851 R0375:A10

SEQ ID NO:451 is the determined cDNA sequence for clone 62116853 R0375:A12

SEQ ID NO:452 is the determined cDNA sequence for clone 62116855 R0375:B02

SEQ ID NO:453 is the determined cDNA sequence for clone 62116856 R0375:B03

SEQ ID NO:454 is the determined cDNA sequence for clone 62116857 R0375:B04

SEQ ID NO:455 is the determined cDNA sequence for clone 62116858 R0375:B05

SEQ ID NO:456 is the determined cDNA sequence for clone 62116859 R0375:B06

SEQ ID NO:457 is the determined cDNA sequence for clone 62116860 R0375:B07

SEQ ID NO:458 is the determined cDNA sequence for clone 62116861 R0375:B08

SEQ ID NO:459 is the determined cDNA sequence for clone 62116862 R0375:B09

SEQ ID NO:460 is the determined cDNA sequence for clone 62116863 R0375:B10

SEQ ID NO:461 is the determined cDNA sequence for clone 62116866 R0375:C01

SEQ ID NO:462 is the determined cDNA sequence for clone 62116867 R0375:C02

SEQ ID NO:463 is the determined cDNA sequence for clone 62116868 R0375:C03

SEQ ID NO:464 is the determined cDNA sequence for clone 62116869 R0375:C04

SEQ ID NO:465 is the determined cDNA sequence for clone 62116870 R0375:C05

SEQ ID NO:466 is the determined cDNA sequence for clone 62116872 R0375:C07

SEQ ID NO:467 is the determined cDNA sequence for clone 62116873 R0375:C08

SEQ ID NO:468 is the determined cDNA sequence for clone 62116874 R0375:C09

SEQ ID NO:469 is the determined cDNA sequence for clone 62116875 R0375:C10

SEQ ID NO:470 is the determined cDNA sequence for clone 62116876 R0375:C11

SEQ ID NO:471 is the determined cDNA sequence for clone 62116877 R0375:C12

SEQ ID NO:472 is the determined cDNA sequence for clone 62116879 R0375:D02

SEQ ID NO:473 is the determined cDNA sequence for clone 62116880 R0375:D03

SEQ ID NO:474 is the determined cDNA sequence for clone 62116881 R0375:D04

SEQ ID NO:475 is the determined cDNA sequence for clone 62116882 R0375:D05

SEQ ID NO:476 is the determined cDNA sequence for clone 62116883 R0375:D06

SEQ ID NO:477 is the determined cDNA sequence for clone 62116884 R0375:D07

SEQ ID NO:478 is the determined cDNA sequence for clone 62116885 R0375:D08

SEQ ID NO:479 is the determined cDNA sequence for clone 62116886 R0375:D09

SEQ ID NO:480 is the determined cDNA sequence for clone 62116887 R0375:D10

SEQ ID NO:481 is the determined cDNA sequence for clone 62116888 R0375:D11

SEQ ID NO:482 is the determined cDNA sequence for clone 62116890 R0375:E01

SEQ ID NO:483 is the determined cDNA sequence for clone 62116891 R0375:E02

SEQ ID NO:484 is the determined cDNA sequence for clone 62116892 R0375:E03

SEQ ID NO:485 is the determined cDNA sequence for clone 62116893 R0375:E04

SEQ ID NO:486 is the determined cDNA sequence for clone 62116894 R0375:E05

SEQ ID NO:487 is the determined cDNA sequence for clone 62116895 R0375:E06

SEQ ID NO:488 is the determined cDNA sequence for clone 62116896 R0375:E07

SEQ ID NO:489 is the determined cDNA sequence for clone 62116897 R0375:E08

SEQ ID NO:490 is the determined cDNA sequence for clone 62116900 R0375:E11

SEQ ID NO:491 is the determined cDNA sequence for clone 62116901 R0375:E12

SEQ ID NO:492 is the determined cDNA sequence for clone 62116902 R0375:F01

SEQ ID NO:493 is the determined cDNA sequence for clone 62116904 R0375:F03

SEQ ID NO:494 is the determined cDNA sequence for clone 62116905 R0375:F04

SEQ ID NO:495 is the determined cDNA sequence for clone 62116906 R0375:F05

SEQ ID NO:496 is the determined cDNA sequence for clone 62116907 R0375:F06

SEQ ID NO:497 is the determined cDNA sequence for clone 62116908 R0375:F07

SEQ ID NO:498 is the determined cDNA sequence for clone 62116909 R0375:F08

SEQ ID NO:499 is the determined cDNA sequence for clone 62116912 R0375:F11

SEQ ID NO:500 is the determined cDNA sequence for clone 62116913 R0375:F12

SEQ ID NO:501 is the determined cDNA sequence for clone 62116914 R0375:G01

SEQ ID NO:502 is the determined cDNA sequence for clone 62116915 R0375:G02

SEQ ID NO:503 is the determined cDNA sequence for clone 62116916 R0375:G03

SEQ ID NO:504 is the determined cDNA sequence for clone 62116917 R0375:G04

SEQ ID NO:505 is the determined cDNA sequence for clone 62116918 R0375:G:05

SEQ ID NO:506 is the determined cDNA sequence for clone 62116919 R0375:G06

SEQ ID NO:507 is the determined cDNA sequence for clone 62116920 R0375:G07

SEQ ID NO:508 is the determined cDNA sequence for clone 62116921 R0375:G08

SEQ ID NO:509 is the determined cDNA sequence for clone 62116922 R0375:G09

SEQ ID NO:510 is the determined cDNA sequence for clone 62116923 R0375:G10

SEQ ID NO:511 is the determined cDNA sequence for clone 62116924 R0375:G11

SEQ ID NO:512 is the determined cDNA sequence for clone 62116925 R0375:G12

SEQ ID NO:513 is the determined cDNA sequence for clone 62116926 R0375:H01

SEQ ID NO:514 is the determined cDNA sequence for clone 62116928 R0375:H03

SEQ ID NO:515 is the determined cDNA sequence for clone 62116929 R0375:H04

SEQ ID NO:516 is the determined cDNA sequence for clone 62116930 R0375:H05

SEQ ID NO:517 is the determined cDNA sequence for clone 62116931 R0375:H06

SEQ ID NO:518 is the determined cDNA sequence for clone 62116934 R0375:H09

SEQ ID NO:519 is the determined cDNA sequence for clone 62116935 R0375:H10

SEQ ID NO:520 is the determined cDNA sequence for clone 62116937 R0376:A02

SEQ ID NO:521 is the determined cDNA sequence for clone 62116939 R0376:A05

SEQ ID NO:522 is the determined cDNA sequence for clone 62116940 R0376:A06

SEQ ID NO:523 is the determined cDNA sequence for clone 62116942 R0376:A08

SEQ ID NO:524 is the determined cDNA sequence for clone 62116943 R0376:A09

SEQ ID NO:525 is the determined cDNA sequence for clone 62116944 R0376:A10

SEQ ID NO:526 is the determined cDNA sequence for clone 62116946 R0376:A12

SEQ ID NO:527 is the determined cDNA sequence for clone 62116948 R0376:B02

SEQ ID NO:528 is the determined cDNA sequence for clone 62116949 R0376:B03

SEQ ID NO:529 is the determined cDNA sequence for clone 62116950 R0376:B04

SEQ ID NO:530 is the determined cDNA sequence for clone 62116951 R0376:B:05

SEQ ID NO:531 is the determined cDNA sequence for clone 62116952 R0376:B06

SEQ ID NO:532 is the determined cDNA sequence for clone 62116953 R0376:B07

SEQ ID NO:533 is the determined cDNA sequence for clone 62116954 R0376:B08

SEQ ID NO:534 is the determined cDNA sequence for clone 62116956 R0376:B10

SEQ ID NO:535 is the determined cDNA sequence for clone 62116957 R0376:B11

SEQ ID NO:536 is the determined cDNA sequence for clone 62116958 R0376:B12

SEQ ID NO:537 is the determined cDNA sequence for clone 62116959 R0376:C01

SEQ ID NO:538 is the determined cDNA sequence for clone 62116960 R0376:C02

SEQ ID NO:539 is the determined cDNA sequence for clone 62116961 R0376:C03

SEQ ID NO:540 is the determined cDNA sequence for clone 62116962 R0376:C04

SEQ ID NO:541 is the determined cDNA sequence for clone 62116963 R0376:C05

SEQ ID NO:542 is the determined cDNA sequence for clone 62116964 R0376:C06

SEQ ID NO:543 is the determined cDNA sequence for clone 62116965 R0376:C07

SEQ ID NO:544 is the determined cDNA sequence for clone 62116966 R0376:C08

SEQ ID NO:545 is the determined cDNA sequence for clone 62116967 R0376:C09

SEQ ID NO:546 is the determined cDNA sequence for clone 62116968 R0376:C10

SEQ ID NO:547 is the determined cDNA sequence for clone 62116969 R0376:C11

SEQ ID NO:548 is the determined cDNA sequence for clone 62116970 R0376:C12

SEQ ID NO:549 is the determined cDNA sequence for clone 62116971 R0376:D01

SEQ ID NO:550 is the determined cDNA sequence for clone 62116972 R0376:D02

SEQ ID NO:551 is the determined cDNA sequence for clone 62116973 R0376:D03

SEQ ID NO:552 is the determined cDNA sequence for clone 62116974 R0376:D04

SEQ ID NO:553 is the determined cDNA sequence for clone 62116975 R0376:D05

SEQ ID NO:554 is the determined cDNA sequence for clone 62116976 R0376:D06

SEQ ID NO:555 is the determined cDNA sequence for clone 62116977 R0376:D07

SEQ ID NO:556 is the determined cDNA sequence for clone 62116978 R0376:D08

SEQ ID NO:557 is the determined cDNA sequence for clone 62116979 R0376:D09

SEQ ID NO:558 is the determined cDNA sequence for clone 62116980 R0376:D10

SEQ ID NO:559 is the determined cDNA sequence for clone 62116981 R0376:D11

SEQ ID NO:560 is the determined cDNA sequence for clone 62116982 R0376:D12

SEQ ID NO:561 is the determined cDNA sequence for clone 62116983 R0376:E01

SEQ ID NO:562 is the determined cDNA sequence for clone 62116984 R0376:E02

SEQ ID NO:563 is the determined cDNA sequence for clone 62116985 R0376:E03

SEQ ID NO:564 is the determined cDNA sequence for clone 62116986 R0376:E04

SEQ ID NO:565 is the determined cDNA sequence for clone 62116987 R0376:E05

SEQ ID NO:566 is the determined cDNA sequence for clone 62116988 R0376:E06

SEQ ID NO:567 is the determined cDNA sequence for clone 62116989 R0376:E07

SEQ ID NO:568 is the determined cDNA sequence for clone 62116990 R0376:E08

SEQ ID NO:569 is the determined cDNA sequence for clone 62116991 R0376:E09

SEQ ID NO:570 is the determined cDNA sequence for clone 62116992 R0376:E10

SEQ ID NO:571 is the determined cDNA sequence for clone 62116993 R0376:E11

SEQ ID NO:572 is the determined cDNA sequence for clone 62116994 R0376:E12

SEQ ID NO:573 is the determined cDNA sequence for clone 62116995 R0376:F01

SEQ ID NO:574 is the determined cDNA sequence for clone 62116996 R0376:F02

SEQ ID NO:575 is the determined cDNA sequence for clone 62116997 R0376:F03

SEQ ID NO:576 is the determined cDNA sequence for clone 62116999 R0376:F05

SEQ ID NO:577 is the determined cDNA sequence for clone 62117000 R0376:F06

SEQ ID NO:578 is the determined cDNA sequence for clone 62117001 R0376:F07

SEQ ID NO:579 is the determined cDNA sequence for clone 62117002 R0376:F08

SEQ ID NO:580 is the determined cDNA sequence for clone 62117003 R0376:F09

SEQ ID NO:581 is the determined cDNA sequence for clone 62117004 R0376:F10

SEQ ID NO:582 is the determined cDNA sequence for clone 62117005 R0376:F11

SEQ ID NO:583 is the determined cDNA sequence for clone 62117006 R0376:F12

SEQ ID NO:584 is the determined cDNA sequence for clone 62117007 R0376:G01

SEQ ID NO:585 is the determined cDNA sequence for clone 62117008 R0376:G02

SEQ ID NO:586 is the determined cDNA sequence for clone 62117009 R0376:G03

SEQ ID NO:587 is the determined cDNA sequence for clone 62117010 R0376:G04

SEQ ID NO:588 is the determined cDNA sequence for clone 62117011 R0376:G05

SEQ ID NO:589 is the determined cDNA sequence for clone 62117012 R0376:G06

SEQ ID NO:590 is the determined cDNA sequence for clone 62117013 R0376:G07

SEQ ID NO:591 is the determined cDNA sequence for clone 62117014 R0376:G08

SEQ ID NO:592 is the determined cDNA sequence for clone 62117015 R0376:G09

SEQ ID NO:593 is the determined cDNA sequence for clone 62117016 R0376: G10

SEQ ID NO:594 is the determined cDNA sequence for clone 62117017 R0376:G11

SEQ ID NO:595 is the determined cDNA sequence for clone 62117018 R0376:G12

SEQ ID NO:596 is the determined cDNA sequence for clone 62117019 R0376:H01

SEQ ID NO:597 is the determined cDNA sequence for clone 62117020 R0376:H02

SEQ ID NO:598 is the determined cDNA sequence for clone 62117023 R0376:H05

SEQ ID NO:599 is the determined cDNA sequence for clone 62117024 R0376:H06

SEQ ID NO:600 is the determined cDNA sequence for clone 62117025 R0376:H07

SEQ ID NO:601 is the determined cDNA sequence for clone 62117026 R0376:H08

SEQ ID NO:602 is the determined cDNA sequence for clone 62117027 R0376:H09

SEQ ID NO:603 is the determined cDNA sequence for clone 62117028 R0376:H10

SEQ ID NO:604 is the determined cDNA sequence for clone 62117029 R0376:H11

SEQ ID NO:605 is the determined cDNA sequence for clone 62117123 R0377:A02

SEQ ID NO:606 is the determined cDNA sequence for clone 62117124 R0377:A03

SEQ ID NO:607 is the determined cDNA sequence for clone 62117125 R0377:A05

SEQ ID NO:608 is the determined cDNA sequence for clone 62117126 R0377:A06

SEQ ID NO:609 is the determined cDNA sequence for clone 62117127 R0377:A07

SEQ ID NO:610 is the determined cDNA sequence for clone 62117128 R0377:A08

SEQ ID NO:611 is the determined cDNA sequence for clone 62117129 R0377:A09

SEQ ID NO:612 is the determined cDNA sequence for clone 62117130 R0377:A10

SEQ ID NO:613 is the determined cDNA sequence for clone 62117131 R0377:A11

SEQ ID NO:614 is the determined cDNA sequence for clone 62117133 R0377:B01

SEQ ID NO:615 is the determined cDNA sequence for clone 62117135 R0377:B03

SEQ ID NO:616 is the determined cDNA sequence for clone 62117136 R0377:B04

SEQ ID NO:617 is the determined cDNA sequence for clone 62117137 R0377:B05

SEQ ID NO:618 is the determined cDNA sequence for clone 62117139 R0377:B:07

SEQ ID NO:619 is the determined cDNA sequence for clone 62117140 R0377:B08

SEQ ID NO:620 is the determined cDNA sequence for clone 62117141 R0377:B09

SEQ ID NO:621 is the determined cDNA sequence for clone 62117142 R0377:B10

SEQ ID NO:622 is the determined cDNA sequence for clone 62117143 R0377: B11

SEQ ID NO:623 is the determined cDNA sequence for clone 62117144 R0377:B12

SEQ ID NO:624 is the determined cDNA sequence for clone 62117146 R0377:C02

SEQ ID NO:625 is the determined cDNA sequence for clone 62117148 R0377:C04

SEQ ID NO:626 is the determined cDNA sequence for clone 62117150 R0377:C06

SEQ ID NO:627 is the determined cDNA sequence for clone 62117151 R0377:C07

SEQ ID NO:628 is the determined cDNA sequence for clone 62117153 R0377:C09

SEQ ID NO:629 is the determined cDNA sequence for clone 62117154 R0377:C10

SEQ ID NO:630 is the determined cDNA sequence for clone 62117155 R0377:C11

SEQ ID NO:631 is the determined cDNA sequence for clone 62117156 R0377:C12

SEQ ID NO:632 is the determined cDNA sequence for clone 62117158 R0377:D02

SEQ ID NO:633 is the determined cDNA sequence for clone 62117159 R0377:D03

SEQ ID NO:634 is the determined cDNA sequence for clone 62117160 R0377:D04

SEQ ID NO:635 is the determined cDNA sequence for clone 62117162 R0377:D06

SEQ ID NO:636 is the determined cDNA sequence for clone 62117163 R0377:D07

SEQ ID NO:637 is the determined cDNA sequence for clone 62117164 R0377:D08

SEQ ID NO:638 is the determined cDNA sequence for clone 62117165 R0377:D09

SEQ ID NO:639 is the determined cDNA sequence for clone 62117166 R0377:D10

SEQ ID NO:640 is the determined cDNA sequence for clone 62117167 R0377:D11

SEQ ID NO:641 is the determined cDNA sequence for clone 62117168 R0377:D12

SEQ ID NO:642 is the determined cDNA sequence for clone 62117169 R0377:E01

SEQ ID NO:643 is the determined cDNA sequence for clone 62117170 R0377:E02

SEQ ID NO:644 is the determined cDNA sequence for clone 62117171 R0377:E03

SEQ ID NO:645 is the determined cDNA sequence for clone 62117172 R0377:E04

SEQ ID NO:646 is the determined cDNA sequence for clone 62117173 R0377:E05

SEQ ID NO:647 is the determined cDNA sequence for clone 62117174 R0377:E06

SEQ ID NO:648 is the determined cDNA sequence for clone 62117176 R0377:E08

SEQ ID NO:649 is the determined .DNA sequence for clone 62117177 R0377:E09

SEQ ID NO:650 is the determined cDNA sequence for clone 62117178 R0377:E10

SEQ ID NO:651 is the determined cDNA sequence for clone 62117179 R0377:E11

SEQ ID NO:652 is the determined cDNA sequence for clone 62117180 R0377:E12

SEQ ID NO:653 is the determined .DNA sequence for clone 62117181 R0377:F01

SEQ ID NO:654 is the determined cDNA sequence for clone 62117182 R0377:F02

SEQ ID NO:655 is the determined cDNA sequence for clone 62117184 R0377:F04

SEQ ID NO:656 is the determined cDNA sequence for clone 62117185 R0377:F05

SEQ ID NO:657 is the determined cDNA sequence for clone 62117186 R0377:F06

SEQ ID NO:658 is the determined cDNA sequence for clone 62117187 R0377:F07

SEQ ID NO:659 is the determined cDNA sequence for clone 62117188 R0377:F08

SEQ ID NO:660 is the determined cDNA sequence for clone 62117189 R0377:F09

SEQ ID NO:661 is the determined cDNA sequence for clone 62117190 R0377:F10

SEQ ID NO:662 is the determined cDNA sequence for clone 62117191 R0377:F11

SEQ ID NO:663 is the determined cDNA sequence for clone 62117192 R0377:F12

SEQ ID NO:664 is the determined cDNA sequence for clone 62117193 R0377:G01

SEQ ID NO:665 is the determined cDNA sequence for clone 62117194 R0377:G02

SEQ ID NO:666 is the determined cDNA sequence for clone 62117195 R0377:G03

SEQ ID NO:667 is the determined cDNA sequence for clone 62117196 R0377:G04

SEQ ID NO:668 is the determined cDNA sequence for clone 62117197 R0377:G05

SEQ ID NO:669 is the determined cDNA sequence for clone 62117199 R0377:G07

SEQ ID NO:670 is the determined cDNA sequence for clone 62117200 R0377:G08

SEQ ID NO:671 is the determined cDNA sequence for clone 62117202 R0377:G10

SEQ ID NO:672 is the determined cDNA sequence for clone 62117203 R0377:G11

SEQ ID NO:673 is the determined cDNA sequence for clone 62117204 R0377:G02

SEQ ID NO:674 is the determined cDNA sequence for clone 62117205 R0377:H01

SEQ ID NO:675 is the determined cDNA sequence for clone 62117206 R0377:H02

SEQ ID NO:676 is the determined cDNA sequence for clone 62117207 R0377:H03

SEQ ID NO:677 is the determined cDNA sequence for clone 62117208 R0377:H04

SEQ ID NO:678 is the determined cDNA sequence for clone 62117209 R0377:H05

SEQ ID NO:679 is the determined cDNA sequence for clone 62117210 R0377:H06

SEQ ID NO:680 is the determined cDNA sequence for clone 62117211 R0377:H07

SEQ ID NO:681 is the determined cDNA sequence for clone 62117212 R0377:H08

SEQ ID NO:682 is the determined cDNA sequence for clone 62117213 R0377:H09

SEQ ID NO:683 is the determined cDNA sequence for clone 62117214 R0377:H10

SEQ ID NO:684 is the determined cDNA sequence for clone 62117216 R0378:A02

SEQ ID NO:685 is the determined cDNA sequence for clone 62117217 R0378:A03

SEQ ID NO:686 is the determined cDNA sequence for clone 62117218 R0378:A05

SEQ ID NO:687 is the determined cDNA sequence for clone 62117219 R0378:A06

SEQ ID NO:688 is the determined cDNA sequence for clone 62117220 R0378:A07

SEQ ID NO:689 is the determined cDNA sequence for clone 62117222 R0378:A09

SEQ ID NO:690 is the determined cDNA sequence for clone 62117223 R0378:A10

SEQ ID NO:691 is the determined cDNA sequence for clone 62117224 R0378:A11

SEQ ID NO:692 is the determined cDNA sequence for clone 62117225 R0378:A12

SEQ ID NO:693 is the determined cDNA sequence for clone 62117226 R0378:B01

SEQ ID NO:694 is the determined cDNA sequence for clone 62117227 R0378:B02

SEQ ID NO:695 is the determined cDNA sequence for clone 62117228 R0378:B03

SEQ ID NO:696 is the determined cDNA sequence for clone 62117229 R0378:B04

SEQ ID NO:697 is the determined cDNA sequence for clone 62117230 R0378:B05

SEQ ID NO:698 is the determined cDNA sequence for clone 62117232 R0378:B07

SEQ ID NO:699 is the determined cDNA sequence for clone 62117233 R0378:B08

SEQ ID NO:700 is the determined cDNA sequence for clone 62117234 R0378:B09

SEQ ID NO:701 is the determined cDNA sequence for clone 62117235 R0378:B10

SEQ ID NO:702 is the determined cDNA sequence for clone 62117237 R0378:B12

SEQ ID NO:703 is the determined cDNA sequence for clone 62117238 R0378:C01

SEQ ID NO:704 is the determined cDNA sequence for clone 62117239 R0378:C02

SEQ ID NO:705 is the determined cDNA sequence for clone 62117240 R0378:C03

SEQ ID NO:706 is the determined cDNA sequence for clone 62117241 R0378:C04

SEQ ID NO:707 is the determined cDNA sequence for clone 62117242 R0378:C05

SEQ ID NO:708 is the determined cDNA sequence for clone 62117243 R0378:C06

SEQ ID NO:709 is the determined cDNA sequence for clone 62117244 R0378:C07

SEQ ID NO:710 is the determined cDNA sequence for clone 62117245 R0378:C08

SEQ ID NO:711 is the determined cDNA sequence for clone 62117247 R0378:C10

SEQ ID NO:712 is the determined cDNA sequence for clone 62117248 R0378:C11

SEQ ID NO:713 is the determined cDNA sequence for clone 62117250 R0378:D01

SEQ ID NO:714 is the determined cDNA sequence for clone 62117251 R0378:D02

SEQ ID NO:715 is the determined cDNA sequence for clone 62117252 R0378:D03

SEQ ID NO:716 is the determined cDNA sequence for clone 62117253 R0378:D04

SEQ ID NO:717 is the determined cDNA sequence for clone 62117254 R0378:D05

SEQ ID NO:718 is the determined cDNA sequence for clone 62117255 R0378:D06

SEQ ID NO:719 is the determined cDNA sequence for clone 62117256 R0378:D07

SEQ ID NO:720 is the determined cDNA sequence for clone 62117257 R0378:D08

SEQ ID NO:721 is the determined cDNA sequence for clone 62117258 R0378:D09

SEQ ID NO:722 is the determined cDNA sequence for clone 62117259 R0378:D10

SEQ ID NO:723 is the determined cDNA sequence for clone 62117261 R0378:D12

SEQ ID NO:724 is the determined cDNA sequence for clone 62117262 R0378:E01

SEQ ID NO:725 is the determined cDNA sequence for clone 62117263 R0378:E02

SEQ ID NO:726 is the determined cDNA sequence for clone 62117264 R0378:E03

SEQ ID NO:727 is the determined cDNA sequence for clone 62117265 R0378:E04

SEQ ID NO:728 is the determined cDNA sequence for clone 62117266 R0378:E05

SEQ ID NO:729 is the determined cDNA sequence for clone 62117267 R0378:E06

SEQ ID NO:730 is the determined cDNA sequence for clone 62117268 R0378:E07

SEQ ID NO:731 is the determined cDNA sequence for clone 62117269 R0378:E08

SEQ ID NO:732 is the determined cDNA sequence for clone 62117270 R0378:E09

SEQ ID NO:733 is the determined cDNA sequence for clone 62117271 R0378:E10

SEQ ID NO:734 is the determined cDNA sequence for clone 62117272 R0378:E11

SEQ ID NO:735 is the determined cDNA sequence for clone 62117273 R0378:E12

SEQ ID NO:736 is the determined cDNA sequence for clone 62117274 R0378:F01

SEQ ID NO:737 is the determined cDNA sequence for clone 62117275 R0378:F02

SEQ ID NO:738 is the determined cDNA sequence for clone 62117276 R0378:F03

SEQ ID NO:739 is the determined cDNA sequence for clone 62117277 R0378:F04

SEQ ID NO:740 is the determined cDNA sequence for clone 62117278 R0378:F05

SEQ ID NO:741 is the determined cDNA sequence for clone 62117279 R0378:F06

SEQ ID NO:742 is the determined cDNA sequence for clone 62117280 R0378:F07

SEQ ID NO:743 is the determined cDNA sequence for clone 62117281 R0378:F08

SEQ ID NO:744 is the determined cDNA sequence for clone 62117282 R0378:F09

SEQ ID NO:745 is the determined cDNA sequence for clone 62117283 R0378:F10

SEQ ID NO:746 is the determined cDNA sequence for clone 62117285 R0378:F12

SEQ ID NO:747 is the determined cDNA sequence for clone 62117287 R0378:G02

SEQ ID NO:748 is the determined cDNA sequence for clone 62117288 R0378:G03

SEQ ID NO:749 is the determined cDNA sequence for clone 62117289 R0378:G04

SEQ ID NO:750 is the determined cDNA sequence for clone 62117290 R0378:G05

SEQ ID NO:751 is the determined cDNA sequence for clone 62117291 R0378:G06

SEQ ID NO:752 is the determined cDNA sequence for clone 62117292 R0378:G07

SEQ ID NO:753 is the determined cDNA sequence for clone 62117293 R0378:G08

SEQ ID NO:754 is the determined cDNA sequence for clone 62117294 R0378:G09

SEQ ID NO:755 is the determined cDNA sequence for clone 62117295 R0378:G10

SEQ ID NO:756 is the determined cDNA sequence for clone 62117296 R0378:G11

SEQ ID NO:757 is the determined cDNA sequence for clone 62117297 R0378:G12

SEQ ID NO:758 is the determined cDNA sequence for clone 62117298 R0378:H01

SEQ ID NO:759 is the determined cDNA sequence for clone 62117299 R0378:H02

SEQ ID NO:760 is the determined cDNA sequence for clone 62117300 R0378:H03

SEQ ID NO:761 is the determined cDNA sequence for clone 62117301 R0378:H04

SEQ ID NO:762 is the determined cDNA sequence for clone 62117302 R0378:H05

SEQ ID NO:763 is the determined cDNA sequence for clone 62117303 R0378:H:06

SEQ ID NO:764 is the determined cDNA sequence for clone 62117304 R0378:H07

SEQ ID NO:765 is the determined cDNA sequence for clone 62117305 R0378:H08

SEQ ID NO:766 is the determined cDNA sequence for clone 62117306 R0378:H09

SEQ ID NO:767 is the determined cDNA sequence for clone 62117307 R0378:H10

SEQ ID NO:768 is the determined cDNA sequence for clone 62117308 R0378:H11

SEQ ID NO:769 is the determined cDNA sequence for clone 62111835 R0379:A02

SEQ ID NO:770 is the determined cDNA sequence for clone 62111836 R0379:A03

SEQ ID NO:771 is the determined cDNA sequence for clone 62111837 R0379:A05

SEQ ID NO:772 is the determined cDNA sequence for clone 62111838 R0379:A06

SEQ ID NO:773 is the determined cDNA sequence for clone 62111839 R0379:A07

SEQ ID NO:774 is the determined cDNA sequence for clone 62111840 R0379:A08

SEQ ID NO:775 is the determined cDNA sequence for clone 62111841 R0379:A09

SEQ ID NO:776 is the determined cDNA sequence for clone 62111842 R0379:A10

SEQ ID NO:777 is the determined cDNA sequence for clone 62111843 R0379:A11

SEQ ID NO:778 is the determined cDNA sequence for clone 62111844 R0379:A12

SEQ ID NO:779 is the determined cDNA sequence for clone 62111846 R0379:B02

SEQ ID NO:780 is the determined cDNA sequence for clone 62111847 R0379:B03

SEQ ID NO:781 is the determined cDNA sequence for clone 62111848 R0379:B04

SEQ ID NO:782 is the determined cDNA sequence for clone 62111849 R0379:B05

SEQ ID NO:783 is the determined cDNA sequence for clone 62111850 R0379:B06

SEQ ID NO:784 is the determined DNA sequence for clone 62111851 R0379:B07

SEQ ID NO:785 is the determined cDNA sequence for clone 62111852 R0379:B08

SEQ ID NO:786 is the determined cDNA sequence for clone 62111854 R0379:B10

SEQ ID NO:787 is the determined cDNA sequence for clone 62111855 R0379:B11

SEQ ID NO:788 is the determined cDNA sequence for clone 62111857 R0379:C01

SEQ ID NO:789 is the determined cDNA sequence for clone 62111858 R0379:C02

SEQ ID NO:790 is the determined cDNA sequence for clone 62111859 R0379:C03

SEQ ID NO:791 is the determined cDNA sequence for clone 62111860 R0379:C04

SEQ ID NO:792 is the determined cDNA sequence for clone 62111861 R0379:C05

SEQ ID NO:793 is the determined cDNA sequence for clone 62111864 R0379:C08

SEQ ID NO:794 is the determined cDNA sequence for clone 62111865 R0379:C09

SEQ ID NO:795 is the determined cDNA sequence for clone 62111866 R0379:C10

SEQ ID NO:796 is the determined cDNA sequence for clone 62111867 R0379:C11

SEQ ID NO:797 is the determined cDNA sequence for clone 62111869 R0379:D01

SEQ ID NO:798 is the determined cDNA sequence for clone 62111870 R0379:D02

SEQ ID NO:799 is the determined cDNA sequence for clone 62111872 R0379:D04

SEQ ID NO:800 is the determined cDNA sequence for clone 62111873 R0379:D05

SEQ ID NO:801 is the determined cDNA sequence for clone 62111874 R0379:D06

SEQ ID NO:802 is the determined cDNA sequence for clone 62111875 R0379:D07

SEQ ID NO:803 is the determined cDNA sequence for clone 62111876 R0379:D08

SEQ ID NO:804 is the determined cDNA sequence for clone 62111877 R0379:D09

SEQ ID NO:805 is the determined cDNA sequence for clone 62111878 R0379:D10

SEQ ID NO:806 is the determined cDNA sequence for clone 62111879 R0379:D11

SEQ ID NO:807 is the determined cDNA sequence for clone 62111881 R0379:E01

SEQ ID NO:808 is the determined cDNA sequence for clone 62111882 R0379:E02

SEQ ID NO:809 is the determined cDNA sequence for clone 62111883 R0379:E03

SEQ ID NO:810 is the determined cDNA sequence for clone 62111884 R0379:E04

SEQ ID NO:811 is the determined cDNA sequence for clone 62111885 R0379:E05

SEQ ID NO:812 is the determined cDNA sequence for clone 62111886 R0379:E06

SEQ ID NO:813 is the determined cDNA sequence for clone 62111887 R0379:E07

SEQ ID NO:814 is the determined cDNA sequence for clone 62111888 R0379:E08

SEQ ID NO:815 is the determined cDNA sequence for clone 62111889 R0379:E09

SEQ ID NO:816 is the determined cDNA sequence for clone 62111890 R0379:E10

SEQ ID NO:817 is the determined cDNA sequence for clone 62111891 R0379:E11

SEQ ID NO:818 is the determined cDNA sequence for clone 62111892 R0379:E12

SEQ ID NO:819 is the determined cDNA sequence for clone 62111893 R0379:F01

SEQ ID NO:820 is the determined cDNA sequence for clone 62111894 R0379:F02

SEQ ID NO:821 is the determined cDNA sequence for clone 62111895 R0379:F03

SEQ ID NO:822 is the determined cDNA sequence for clone 62111896 R0379:F04

SEQ ID NO:823 is the determined cDNA sequence for clone 62111897 R0379:F05

SEQ ID NO:824 is the determined cDNA sequence for clone 62111898 R0379:F06

SEQ ID NO:825 is the determined cDNA sequence for clone 62111899 R0379:F07

SEQ ID NO:826 is the determined cDNA sequence for clone 62111900 R0379:F08

SEQ ID NO:827 is the determined cDNA sequence for clone 62111901 R0379:F09

SEQ ID NO:828 is the determined cDNA sequence for clone 62111902 R0379:F10

SEQ ID NO:829 is the determined cDNA sequence for clone 62111903 R0379:F11

SEQ ID NO:830 is the determined cDNA sequence for clone 62111904 R0379:F12

SEQ ID NO:831 is the determined cDNA sequence for clone 62111905 R0379:G01

SEQ ID NO:832 is the determined cDNA sequence for clone 62111906 R0379:G02

SEQ ID NO:833 is the determined cDNA sequence for clone 62111907 R0379:G03

SEQ ID NO:834 is the determined cDNA sequence for clone 62111908 R0379:G04

SEQ ID NO:835 is the determined cDNA sequence for clone 62111909 R0379:G05

SEQ ID NO:836 is the determined cDNA sequence for clone 62111910 R0379:G06

SEQ ID NO:837 is the determined cDNA sequence for clone 62111911 R0379:G07

SEQ ID NO:838 is the determined cDNA sequence for clone 62111912 R0379:G08

SEQ ID NO:839 is the determined cDNA sequence for clone 62111913 R0379:G09

SEQ ID NO:840 is the determined cDNA sequence for clone 62111914 R0379:G10

SEQ ID NO:841 is the determined cDNA sequence for clone 62111916 R0379:G12

SEQ ID NO:842 is the determined cDNA sequence for clone 62111917 R0379:H01

SEQ ID NO:843 is the determined cDNA sequence for clone 62111918 R0379:H02

SEQ ID NO:844 is the determined cDNA sequence for clone 62111919 R0379:H03

SEQ ID NO:845 is the determined cDNA sequence for clone 62111920 R0379:H04

SEQ ID NO:846 is the determined cDNA sequence for clone 62111921 R0379:H05

SEQ ID NO:847 is the determined cDNA sequence for clone 62111922 R0379:H:06

SEQ ID NO:848 is the determined cDNA sequence for clone 62111923 R0379:H07

SEQ ID NO:849 is the determined cDNA sequence for clone 62111924 R0379:H08

SEQ ID NO:850 is the determined cDNA sequence for clone 62111925 R0379:H09

SEQ ID NO:851 is the determined cDNA sequence for clone 62111926 R0379:H10

SEQ ID NO:852 is the determined cDNA sequence for clone 62111927 R0379:H11

SEQ ID NO:853 is the determined cDNA sequence for clone 62112630 R0380:A02

SEQ ID NO:854 is the determined cDNA sequence for clone 62112631 R0380:A03

SEQ ID NO:855 is the determined cDNA sequence for clone 62112632 R0380:A05

SEQ ID NO:856 is the determined cDNA sequence for clone 62112633 R0380:A06

SEQ ID NO:857 is the determined cDNA sequence for clone 62112634 R0380:A07

SEQ ID NO:858 is the determined cDNA sequence for clone 62112635 R0380:A08

SEQ ID NO:859 is the determined cDNA sequence for clone 62112636 R0380:A09

SEQ ID NO:860 is the determined cDNA sequence for clone 62112637 R0380:A10

SEQ ID NO:861 is the determined cDNA sequence for clone 62112639 R0380:A12

SEQ ID NO:862 is the determined cDNA sequence for clone 62112640 R0380:B01

SEQ ID NO:863 is the determined cDNA sequence for clone 62112641 R0380:B02

SEQ ID NO:864 is the determined cDNA sequence for clone 62112642 R0380:B03

SEQ ID NO:865 is the determined cDNA sequence for clone 62112643 R0380:B04

SEQ ID NO:866 is the determined cDNA sequence for clone 62112644 R0380:B05

SEQ ID NO:867 is the determined cDNA sequence for clone 62112645 R0380:B06

SEQ ID NO:868 is the determined cDNA sequence for clone 62112646 R0380:B07

SEQ ID NO:869 is the determined cDNA sequence for clone 62112647 R0380:B08

SEQ ID NO:870 is the determined cDNA sequence for clone 62112648 R0380:B09

SEQ ID NO:871 is the determined cDNA sequence for clone 62112649 R0380:B10

SEQ ID NO:872 is the determined cDNA sequence for clone 62112650 R0380:B11

SEQ ID NO:873 is the determined cDNA sequence for clone 62112652 R0380:C01

SEQ ID NO:874 is the determined cDNA sequence for clone 62112653 R0380:C02

SEQ ID NO:875 is the determined cDNA sequence for clone 62112654 R0380:C03

SEQ ID NO:876 is the determined cDNA sequence for clone 62112655 R0380:C04

SEQ ID NO:877 is the determined cDNA sequence for clone 62112656 R0380:C05

SEQ ID NO:878 is the determined cDNA sequence for clone 62112658 R0380:C07

SEQ ID NO:879 is the determined cDNA sequence for clone 62112659 R0380:C08

SEQ ID NO:880 is the determined cDNA sequence for clone 62112660 R0380:C09

SEQ ID NO:881 is the determined cDNA sequence for clone 62112661 R0380:C10

SEQ ID NO:882 is the determined cDNA sequence for clone 62112662 R0380:C11

SEQ ID NO:883 is the determined cDNA sequence for clone 62112663 R0380:C12

SEQ ID NO:884 is the determined cDNA sequence for clone 62112664 R0380:D01

SEQ ID NO:885 is the determined cDNA sequence for clone 62112665 R0380:D02

SEQ ID NO:886 is the determined cDNA sequence for clone 62112666 R0380:D03

SEQ ID NO:887 is the determined cDNA sequence for clone 62112667 R0380:D04

SEQ ID NO:888 is the determined cDNA sequence for clone 62112668 R0380:D05

SEQ ID NO:889 is the determined cDNA sequence for clone 62112669 R0380:D06

SEQ ID NO:890 is the determined cDNA sequence for clone 62112670 R0380:D07

SEQ ID NO:891 is the determined cDNA sequence for clone 62112671 R0380:D08

SEQ ID NO:892 is the determined cDNA sequence for clone 62112673 R0380:D10

SEQ ID NO:893 is the determined cDNA sequence for clone 62112674 R0380:D11

SEQ ID NO:894 is the determined cDNA sequence for clone 62112675 R0380:D12

SEQ ID NO:895 is the determined cDNA sequence for clone 62112676 R0380:E01

SEQ ID NO:896 is the determined cDNA sequence for clone 62112677 R0380:E02

SEQ ID NO:897 is the determined cDNA sequence for clone 62112678 R0380:E03

SEQ ID NO:898 is the determined cDNA sequence for clone 62112679 R0380:E04

SEQ ID NO:899 is the determined cDNA sequence for clone 62112681 R0380:E06

SEQ ID NO:900 is the determined cDNA sequence for clone 62112683 R0380:E08

SEQ ID NO:901 is the determined cDNA sequence for clone 62112684 R0380:E09

SEQ ID NO:902 is the determined cDNA sequence for clone 62112685 R0380:E10

SEQ ID NO:903 is the determined cDNA sequence for clone 62112686 R0380:E11

SEQ ID NO:904 is the determined cDNA sequence for clone 62112687 R0380:E12

SEQ ID NO:905 is the determined cDNA sequence for clone 62112688 R0380:F01

SEQ ID NO:906 is the determined cDNA sequence for clone 62112689 R0380:F02

SEQ ID NO:907 is the determined cDNA sequence for clone 62112690 R0380:F03

SEQ ID NO:908 is the determined cDNA sequence for clone 62112691 R0380:F04

SEQ ID NO:909 is the determined cDNA sequence for clone 62112692 R0380:F05

SEQ ID NO:910 is the determined cDNA sequence for clone 62112693 R0380:F06

SEQ ID NO:911 is the determined cDNA sequence for clone 62112694 R0380:F07

SEQ ID NO:912 is the determined cDNA sequence for clone 62112695 R0380:F08

SEQ ID NO:913 is the determined cDNA sequence for clone 62112696 R0380:F09

SEQ ID NO:914 is the determined cDNA sequence for clone 62112697 R0380:F10

SEQ ID NO:915 is the determined cDNA sequence for clone 62112698 R0380:F11

SEQ ID NO:916 is the determined cDNA sequence for clone 62112699 R0380:F12

SEQ ID NO:917 is the determined cDNA sequence for clone 62112700 R0380:G01

SEQ ID NO:918 is the determined cDNA sequence for clone 62112701 R0380:G02

SEQ ID NO:919 is the determined cDNA sequence for clone 62112702 R0380:G03

SEQ ID NO:920 is the determined cDNA sequence for clone 62112703 R0380:G04

SEQ ID NO:921 is the determined cDNA sequence for clone 62112704 R0380:G05

SEQ ID NO:922 is the determined cDNA sequence for clone 62112706 R0380:G07

SEQ ID NO:923 is the determined cDNA sequence for clone 62112707 R0380:G08

SEQ ID NO:924 is the determined cDNA sequence for clone 62112708 R0380:G09

SEQ ID NO:925 is the determined cDNA sequence for clone 62112709 R0380:G10

SEQ ID NO:926 is the determined cDNA sequence for clone 62112710 R0380:G11

SEQ ID NO:927 is the determined cDNA sequence for clone 62112711 R0380:G12

SEQ ID NO:928 is the determined cDNA sequence for clone 62112712 R0380:H01

SEQ ID NO:929 is the determined cDNA sequence for clone 62112713 R0380:H02

SEQ ID NO:930 is the determined cDNA sequence for clone 62112714 R0380:H03

SEQ ID NO:931 is the determined cDNA sequence for clone 62112715 R0380:H04

SEQ ID NO:932 is the determined cDNA sequence for clone 62112716 R0380:H05

SEQ ID NO:933 is the determined cDNA sequence for clone 62112717 R0380:H06

SEQ ID NO:934 is the determined cDNA sequence for clone 62112718 R0380:H07

SEQ ID NO:935 is the determined cDNA sequence for clone 62112719 R0380:H08

SEQ ID NO:936 is the determined cDNA sequence for clone 62112720 R0380:H09

SEQ ID NO:937 is the determined cDNA sequence for clone 62112721 R0380:H10

SEQ ID NO:938 is the determined cDNA sequence for clone 62112021 R0381:A02

SEQ ID NO:939 is the determined cDNA sequence for clone 62112022 R0381:A03

SEQ ID NO:940 is the determined cDNA sequence for clone 62112023 R0381:A05

SEQ ID NO:941 is the determined cDNA sequence for clone 62112024 R0380 :A06

SEQ ID NO:942 is the determined cDNA sequence for clone 62112025 R0381:A07

SEQ ID NO:943 is the determined cDNA sequence for clone 62112026 R0381:A08

SEQ ID NO:944 is the determined cDNA sequence for clone 62112027 R0381:A09

SEQ ID NO:945 is the determined cDNA sequence for clone 62112028 R0381:A10

SEQ ID NO:946 is the determined cDNA sequence for clone 62112029 R0381 A11

SEQ ID NO:947 is the determined cDNA sequence for clone 62112031 R0381:B01

SEQ ID NO:948 is the determined cDNA sequence for clone 62112032 R0381:B02

SEQ ID NO:949 is the determined cDNA sequence for clone 62112033 R0381:B03

SEQ ID NO:950 is the determined cDNA sequence for clone 62112034 R0381:B04

SEQ ID NO:951 is the determined cDNA sequence for clone 62112035 R0381:B05

SEQ ID NO:952 is the determined cDNA sequence for clone 62112036 R0381:B06

SEQ ID NO:953 is the determined cDNA sequence for clone 62112037 R0381:B07

SEQ ID NO:954 is the determined cDNA sequence for clone 62112038 R0381:B08

SEQ ID NO:955 is the determined cDNA sequence for clone 62112039 R0381:B09

SEQ ID NO:956 is the determined cDNA sequence for clone 62112040 R0381:B10

SEQ ID NO:957 is the determined cDNA sequence for clone 62112041 R0381:B11

SEQ ID NO:958 is the determined cDNA sequence for clone 62112042 R0381:B12

SEQ ID NO:959 is the determined cDNA sequence for clone 62112043 R03.8 C01

SEQ ID NO:960 is the determined cDNA sequence for clone 62112044 R0381:C02

SEQ .D NO:961 is the determined cDNA sequence for clone 62112046 R0381:C04

SEQ ID NO:962 is the determined cDNA sequence for clone 62112047 R0381:C05

SEQ ID NO:963 is the determined cDNA sequence for clone 62112049 R0381:C07

SEQ ID NO:964 is the determined cDNA sequence for clone 62112050 R0381:C08

SEQ ID NO:965 is the determined cDNA sequence for clone 62112051 R0381:C09

SEQ ID NO:966 is the determined cDNA sequence for clone 62112052 R0381:C10

SEQ ID NO:967 is the determined cDNA sequence for clone 62112053 R0381:C11

SEQ ID NO:968 is the determined cDNA sequence for clone 62112054 R0381:C12

SEQ ID NO:969 is the determined cDNA sequence for clone 62112057 R0381:D03

SEQ ID NO:970 is the determined cDNA sequence for clone 62112058 R0381:D04

SEQ ID NO:971 is the determined cDNA sequence for clone 62112059 R0381:D05

SEQ ID NO:972 is the determined cDNA sequence for clone 62112060 R0381:D06

SEQ ID NO:973 is the determined cDNA sequence for clone 62112061 R0381:D07

SEQ ID NO:974 is the determined cDNA sequence for clone 62112062 R0381:D08

SEQ ID NO:975 is the determined cDNA sequence for clone 62112063 R0381:D09

SEQ ID NO:976 is the determined cDNA sequence for clone 62112064 R0381:D10

SEQ ID NO:977 is the determined cDNA sequence for clone 62112065 R0381:D11

SEQ ID NO:978 is the determined cDNA sequence for clone 62112066 R0381:D12

SEQ ID NO:979 is the determined cDNA sequence for clone 62112067 R0381:E01

SEQ ID NO:980 is the determined cDNA sequence for clone 62112068 R0381:E02

SEQ ID NO:981 is the determined cDNA sequence for clone 62112069 R0381:E03

SEQ ID NO:982 is the determined cDNA sequence for clone 62112070 R0381:E04

SEQ ID NO:983 is the determined cDNA sequence for clone 62112071 R0381:E05

SEQ ID NO:984 is the determined cDNA sequence for clone 62112072 R0381:E06

SEQ ID NO:985 is the determined cDNA sequence for clone 62112073 R0381:E07

SEQ ID NO:986 is the determined cDNA sequence for clone 62112074 R0381:E08

SEQ ID NO:987 is the determined cDNA sequence for clone 62112075 R0381:E09

SEQ ID NO:988 is the determined cDNA sequence for clone 62112076 R0381:E10

SEQ ID NO:989 is the determined cDNA sequence for clone 62112077 R0381:E11

SEQ ID NO:990 is the determined cDNA sequence for clone 62112078 R0381:E12

SEQ ID NO:991 is the determined cDNA sequence for clone 62112079 R0381:F01

SEQ ID NO:992 is the determined cDNA sequence for clone 62112080 R0381.:F02

SEQ ID NO:993 is the determined cDNA sequence for clone 62112081 R0381:F03

SEQ ID NO:994 is the determined cDNA sequence for clone 62112082 R0381:F04

SEQ ID NO:995 is the determined cDNA sequence for clone 62112083 R0381:F05

SEQ ID NO:996 is the determined cDNA sequence for clone 62112084 R03.8 .:F06

SEQ ID NO:997 is the determined cDNA sequence for clone 62112085 R0381:F07

SEQ ID NO:998 is the determined cDNA sequence for clone 62112086 R0381:F8

SEQ ID NO:999 is the determined cDNA sequence for clone 62112087 R0381:F09

SEQ ID NO:1000 is the determined cDNA sequence for clone 62112088 R0381:F10

SEQ ID NO:1001 is the determined cDNA sequence for clone 62112089 R0381:F11

SEQ ID NO:1002 is the determined cDNA sequence for clone 62112090 R0381:F12

SEQ ID NO:1003 is the determined cDNA sequence for clone 62112092 R0381: G02

SEQ ID NO:1004 is the determined cDNA sequence for clone 62112093 R0381:G03

SEQ ID NO:1005 is the determined cDNA sequence for clone 62112095 R0381:G05

SEQ ID NO:1006 is the determined cDNA sequence for clone 62112096 R0381 : G06

SEQ ID NO:1007 is the determined cDNA sequence for clone 62112097 R0381:G07

SEQ ID NO:1008 is the determined cDNA sequence for clone 62112098 R0381:G08

SEQ ID NO:1009 is the determined cDNA sequence for clone 62112099 R0381:G09

SEQ ID NO:1010 is the determined cDNA sequence for clone 62112100 R0381:G10

SEQ ID NO:1011 is the determined cDNA sequence for clone 62112101 R0381:G11

SEQ ID NO:1012 is the determined cDNA sequence for clone 62112102 R0381:G12

SEQ ID NO:1013 is the determined cDNA sequence for clone 62112103 R0381:H01

SEQ ID NO:1014 is the determined cDNA sequence for clone 62112104 R0381:H02

SEQ ID NO:1015 is the determined cDNA sequence for clone 62112105 R0381:H03

SEQ ID NO:1016 is the determined cDNA sequence for clone 62112106 R0381:H04

SEQ ID NO:1017 is the determined cDNA sequence for clone 62112107 R0381:H05

SEQ ID NO:1018 is the determined cDNA sequence for clone 62112108 R0381:H06

SEQ ID NO:1019 is the determined cDNA sequence for clone 62112109 R0381:H07

SEQ ID NO:1020 is the determined cDNA sequence for clone 62112110 R0381:H08

SEQ ID NO:1021 is the determined cDNA sequence for clone 62112111 R0381:H09

SEQ ID NO:1022 is the determined cDNA sequence for clone 62112112 R0381:H10

SEQ ID NO:1023 is the determined cDNA sequence for clone 62112113 R03:8 :H11

SEQ ID NO:1024 is the determined cDNA sequence for clone 62117402 R0382:A02

SEQ ID NO:1025 is the determined cDNA sequence for clone 62117403 R0382:A03

SEQ ID NO:1026 is the determined cDNA sequence for clone 62117405 R0382:A06

SEQ ID NO:1027 is the determined cDNA sequence for clone 62117406 R0382:A07

SEQ ID NO:1028 is the determined cDNA sequence for clone 62117407 R0382:A08

SEQ ID NO:1029 is the determined cDNA sequence for clone 62117408 R0382:A09

SEQ ID NO:1030 is the determined cDNA sequence for clone 62117409 R0382:A10

SEQ ID NO:1031 is the determined cDNA sequence for clone 62117410 R0382:A11

SEQ ID NO:1032 is the determined cDNA sequence for clone 62117411 R0382:A12

SEQ ID NO:1033 is the determined cDNA sequence for clone 62117412 R0382:B01

SEQ ID NO:1034 is the determined cDNA sequence for clone 62117413 R0382:B02

SEQ ID NO:1035 is the determined cDNA sequence for clone 62117414 R0382:B03

SEQ ID NO:1036 is the determined cDNA sequence for clone 62117415 R0382:B04

SEQ ID NO:1037 is the determined cDNA sequence for clone 62117416 R0382:B05

SEQ ID NO:1038 is the determined cDNA sequence for clone 62117418 R0382:B07

SEQ ID NO:1039 is the determined cDNA sequence for clone 62117419 R0382:B08

SEQ ID NO:1040 is the determined cDNA sequence for clone 62117420 R0382:B09

SEQ ID NO:1041 is the determined cDNA sequence for clone 62117421 R0382:B10

SEQ ID NO:1042 is the determined cDNA sequence for clone 62117422 R0382:B11

SEQ ID NO:1043 is the determined cDNA sequence for clone 62117423 R0382:B12

SEQ ID NO:1044 is the determined cDNA sequence for clone 62117424 R0382:C01

SEQ ID NO:1045 is the determined cDNA sequence for clone 62117425 R0382:C02

SEQ ID NO:1046 is the determined cDNA sequence for clone 62117426 R0382:C03

SEQ ID NO:1047 is the determined cDNA sequence for clone 62117427 R0382:C04

SEQ ID NO:1048 is the determined cDNA sequence for clone 62117429 R0382:C06

SEQ ID NO:1049 is the determined cDNA sequence for clone 62117430 R0382:C07

SEQ ID NO:1050 is the determined cDNA sequence for clone 62117432 R0382:C09

SEQ ID NO:1051 is the determined cDNA sequence for clone 62117433 R0382:C10

SEQ ID NO:1052 is the determined cDNA sequence for clone 62117434 R0382:C11

SEQ ID NO:1053 is the determined cDNA sequence for clone 62117435 R0382:C12

SEQ ID NO:1054 is the determined cDNA sequence for clone 62117437 R0382:D02

SEQ ID NO:1055 is the determined cDNA sequence for clone 62117438 R0382:D03

SEQ ID NO:1056 is the determined cDNA sequence for clone 62117439 R0382:D04

SEQ ID NO:1057 is the determined cDNA sequence for clone 62117440 R0382:D05

SEQ ID NO:1058 is the determined cDNA sequence for clone 62117441 R0382:D06

SEQ ID NO:1059 is the determined cDNA sequence for clone 62117442 R0382:D07

SEQ ID NO:1060 is the determined cDNA sequence for clone 62117443 R0382:D08

SEQ ID NO:1061 is the determined cDNA sequence for clone 62117444 R0382:D09

SEQ ID NO:1062 is the determined cDNA sequence for clone 62117445 R0382:D10

SEQ ID NO:1063 is the determined cDNA sequence for clone 62117446 R0382:D11

SEQ ID NO:1064 is the determined cDNA sequence for clone 62117447 R0382:D12

SEQ ID NO:1065 is the determined cDNA sequence for clone 62117448 R0382:E01

SEQ ID NO:1066 is the determined cDNA sequence for clone 62117449 R0382:E02

SEQ ID NO:1067 is the determined cDNA sequence for clone 62117450 R0382:E03

SEQ ID NO:1068 is the determined cDNA sequence for clone 62117451 R0382:E04

SEQ ID NO:1069 is the determined cDNA sequence for clone 62117452 R0382:E05

SEQ ID NO:1070 is the determined cDNA sequence for clone 62117453 R0382:E06

SEQ ID NO:1071 is the determined cDNA sequence for clone 62117455 R0382:E08

SEQ ID NO:1072 is the determined cDNA sequence for clone 62117456 R0382:E09

SEQ ID NO:1073 is the determined cDNA sequence for clone 62117457 R0382:E10

SEQ ID NO:1074 is the determined cDNA sequence for clone 62117458 R0382:E11

SEQ ID NO:1075 is the determined cDNA sequence for clone 62117459 R0382:E12

SEQ ID NO:1076 is the determined cDNA sequence for clone 62117460 R0382:F01

SEQ ID NO:1077 is the determined cDNA sequence for clone 62117461 R0382:F02

SEQ ID NO:1078 is the determined cDNA sequence for clone 62117462 R0382: F03

SEQ ID NO:1079 is the determined cDNA sequence for clone 62117463 R0382:F04

SEQ ID NO:1080 is the determined cDNA sequence for clone 62117464 R0382:F05

SEQ ID NO:1081 is the determined cDNA sequence for clone 62117465 R0382:F06

SEQ ID NO:1082 is the determined cDNA sequence for clone 62117466 R0382:F07

SEQ ID NO:1083 is the determined cDNA sequence for clone 62117467 R0382:F08

SEQ ID NO:1084 is the determined cDNA sequence for clone 62117468 R0382:F09

SEQ ID NO:1085 is the determined cDNA sequence for clone 62117469 R0382:F10

SEQ ID NO:1086 is the determined cDNA sequence for clone 62117470 R0382:F11

SEQ ID NO:1087 is the determined cDNA sequence for clone 62117471 R0382:F12

SEQ ID NO:1088 is the determined cDNA sequence for clone 62117472 R0382:G01

SEQ ID NO:1089 is the determined cDNA sequence for clone 62117473 R0382:G02

SEQ ID NO:1090 is the determined cDNA sequence for clone 62117474 R0382:G03

SEQ ID NO:1091 is the determined cDNA sequence for clone 62117475 R0382:G04

SEQ ID NO:1092 is the determined cDNA sequence for clone 62117476 R0382:G05

SEQ ID NO:1093 is the determined cDNA sequence for clone 62117477 R0382:G06

SEQ ID NO:1094 is the determined cDNA sequence for clone 62117478 R0382:G07

SEQ ID NO:1095 is the determined cDNA sequence for clone 62117479 R0382:G08

SEQ ID NO:1096 is the determined cDNA sequence for clone 62117480 R0382:G09

SEQ ID NO:1097 is the determined cDNA sequence for clone 62117482 R0382:G11

SEQ ID NO:1098 is the determined cDNA sequence for clone 62117483 R0382:G12

SEQ ID NO:1099 is the determined cDNA sequence for clone 62117484 R0382:H01

SEQ ID NO:1100 is the determined cDNA sequence for clone 62117485 R0382:H02

SEQ ID NO:1101 is the determined cDNA sequence for clone 62117486 R0382:H03

SEQ ID NO:1102 is the determined cDNA sequence for clone 62117487 R0382:H04

SEQ ID NO:1103 is the determined cDNA sequence for clone 62117488 R0382:H05

SEQ ID NO:1104 is the determined cDNA sequence for clone 62117489 R0382:H06

SEQ ID NO:1105 is the determined cDNA sequence for clone 62117490 R0382:H07

SEQ ID NO:1106 is the determined cDNA sequence for clone 62117491 R0382:H08

SEQ ID NO:1107 is the determined cDNA sequence for clone 62117492 R0382:H09

SEQ ID NO:1108 is the determined cDNA sequence for clone 62117493 R0382:H10

SEQ ID NO:1109 is the determined cDNA sequence for clone 62117494 R0382:H11

SEQ ID NO:1110 is the determined cDNA sequence for clone 62117309 R0383:A02

SEQ ID NO:1111 is the determined cDNA sequence for clone 62117310 R0383:A03

SEQ ID NO:1112 is the determined cDNA sequence for clone 62117311 R0383:A05

SEQ ID NO:1113 is the determined cDNA sequence for clone 62117313 R0383:A07

SEQ ID NO:1114 is the determined cDNA sequence for clone 62117314 R0383:A08

SEQ ID NO:1115 is the determined cDNA sequence for clone 62117316 R0383:A10

SEQ ID NO:1116 is the determined cDNA sequence for clone 62117317 R0383:A11

SEQ ID NO:1117 is the determined cDNA sequence for clone 62117318 R0383:A12

SEQ ID NO:1118 is the determined cDNA sequence for clone 62117319 R0383:B01

SEQ ID NO:1119 is the determined cDNA sequence for clone 62117320 R0383 :B02

SEQ ID NO:1120 is the determined cDNA sequence for clone 62117321 R0383:B03

SEQ ID NO:1121 is the determined cDNA sequence for clone 62117322 R0383:B04

SEQ ID NO:1122 is the determined cDNA sequence for clone 62117323 R0383:B05

SEQ ID NO:1123 is the determined cDNA sequence for clone 62117324 R0383:B06

SEQ ID NO:1124 is the determined cDNA sequence for clone 62117325 R0383:B07

SEQ ID NO:1125 is the determined cDNA sequence for clone 62117326 R0383:B08

SEQ ID NO:1126 is the determined cDNA sequence for clone 62117327 R0383:B09

SEQ ID NO:1127 is the determined cDNA sequence for clone 62117328 R0383:B 10

SEQ ID NO:1128 is the determined cDNA sequence for clone 62117329 R0383:B11

SEQ ID NO:1129 is the determined cDNA sequence for clone 62117330 R0383:B12

SEQ ID NO:1130 is the determined cDNA sequence for clone 62117331 R0383:C01

SEQ ID NO:1131 is the determined cDNA sequence for clone 62117332 R0383:C02

SEQ ID NO:1132 is the determined cDNA sequence for clone 62117333 R0383:C03

SEQ ID NO:1133 is the determined cDNA sequence for clone 62117334 R0383:C04

SEQ ID NO:1134 is the determined cDNA sequence for clone 62117335 R0383:C05

SEQ ID NO:1135 is the determined cDNA sequence for clone 62117336 R0383:C06

SEQ ID NO:1136 is the determined cDNA sequence for clone 62117337 R0383:C07

SEQ ID NO:1137 is the determined cDNA sequence for clone 62117338 R0383:C08

SEQ ID NO:1138 is the determined cDNA sequence for clone 62117339 R0383:C09

SEQ ID NO:1139 is the determined cDNA sequence for clone 62117340 R0383:C10

SEQ ID NO:1140 is the determined cDNA sequence for clone 62117341 R0383:C11

SEQ ID NO:1141 is the determined cDNA sequence for clone 62117342 R0383:C12

SEQ ID NO:1142 is the determined cDNA sequence for clone 62117343 R0383:D01

SEQ ID NO:1143 is the determined cDNA sequence for clone 62117344 R0383:D02

SEQ ID NO:1144 is the determined cDNA sequence for clone 62117345 R0383:D03

SEQ ID NO:1145 is the determined cDNA sequence for clone 62117346 R0383:D04

SEQ ID NO:1146 is the determined cDNA sequence for clone 62117347 R0383:D05

SEQ ID NO:1147 is the determined cDNA sequence for clone 62117348 R0383:D06

SEQ ID NO:1148 is the determined cDNA sequence for clone 62117349 R0383:D07

SEQ ID NO:1149 is the determined cDNA sequence for clone 62117350 R0383:D08

SEQ ID NO:1150 is the determined cDNA sequence for clone 62117351 R0383:D09

SEQ ID NO:1151 is the determined cDNA sequence for clone 62117352 R0383:D10

SEQ ID NO:1152 is the determined cDNA sequence for clone 62117353 R0383:D11

SEQ ID NO:1153 is the determined cDNA sequence for clone 62117354 R0383:D12

SEQ ID NO:1154 is the determined cDNA sequence for clone 62117355 R0383:E01

SEQ ID NO:1155 is the determined cDNA sequence for clone 62117356 R0383:E02

SEQ ID NO:1156 is the determined cDNA sequence for clone 62117357 R0383:E03

SEQ ID NO:1157 is the determined cDNA sequence for clone 62117358 R0383:E04

SEQ ID NO:1158 is the determined cDNA sequence for clone 62117359 R0383:E05

SEQ ID NO:1159 is the determined cDNA sequence for clone 62117360 R0383:E06

SEQ ID NO:1160 is the determined cDNA sequence for clone 62117361 R0383:E07

SEQ ID NO:1161 is the determined cDNA sequence for clone 62117362 R0383:E:08

SEQ ID NO:1162 is the determined cDNA sequence for clone 62117363 R0383:E09

SEQ ID NO:1163 is the determined cDNA sequence for clone 62117364 R0383:E10

SEQ ID NO:1164 is the determined cDNA sequence for clone 62117365 R0383:E11

SEQ ID NO:1165 is the determined cDNA sequence for clone 62117366 R0383:E12

SEQ ID NO:1166 is the determined cDNA sequence for clone 62117367 R0383:F01

SEQ ID NO:1167 is the determined cDNA sequence for clone 62117368 R0383:F02

SEQ ID NO:1168 is the determined cDNA sequence for clone 62117369 R0383:F03

SEQ ID NO:1169 is the determined cDNA sequence for clone 62117370 R0383:F04

SEQ ID NO:1170 is the determined cDNA sequence for clone 62117371 R0383:F05

SEQ ID NO:1171 is the determined cDNA sequence for clone 62117372 R0383:F06

SEQ ID NO:1172 is the determined cDNA sequence for clone 62117373 R0383:F07

SEQ ID NO:1173 is the determined cDNA sequence for clone 62117374 R0383:F08

SEQ ID NO:1174 is the determined cDNA sequence for clone 62117375 R0383:F09

SEQ ID NO:1175 is the determined cDNA sequence for clone 62117376 R0383:F10

SEQ ID NO:1176 is the determined cDNA sequence for clone 62117377 R0383:F11

SEQ ID NO:1177 is the determined cDNA sequence for clone 62117378 R0383:F12

SEQ ID NO:1178 is the determined cDNA sequence for clone 62117379 R0383:G01

SEQ ID NO:1179 is the determined cDNA sequence for clone 62117380 R0383:G02

SEQ ID NO:1180 is the determined cDNA sequence for clone 62117381 R0383:G03

SEQ ID NO:1181 is the determined cDNA sequence for clone 62117382 R0383:G04

SEQ ID NO:1182 is the determined cDNA sequence for clone 62117383 R0383:G05

SEQ ID NO:1183 is the determined cDNA sequence for clone 62117384 R0383:G06

SEQ ID NO:1184 is the determined cDNA sequence for clone 62117385 R0383:G07

SEQ ID NO:1185 is the determined cDNA sequence for clone 62117386 R0383:G08

SEQ ID NO:1186 is the determined cDNA sequence for clone 62117387 R0383:G09

SEQ ID NO:1187 is the determined cDNA sequence for clone 62117388 R0383:G10

SEQ ID NO:1188 is the determined cDNA sequence for clone 62117389 R0383 :G11

SEQ ID NO:1189 is the determined cDNA sequence for clone 62117390 R0383:G12

SEQ ID NO:1190 is the determined cDNA sequence for clone 62117391 R0383:H01

SEQ ID NO:1191 is the determined cDNA sequence for clone 62117392 R0383:H02

SEQ ID NO:1192 is the determined cDNA sequence for clone 62117393 R0383:H03

SEQ ID NO:1193 is the determined cDNA sequence for clone 62117394 R0383:H04

SEQ ID NO:1194 is the determined cDNA sequence for clone 62117395 R0383:H05

SEQ ID NO:1195 is the determined cDNA sequence for clone 62117396 R0383:H06

SEQ ID NO:1196 is the determined cDNA sequence for clone 62117397 R0383:H07

SEQ ID NO:1197 is the determined cDNA sequence for clone 62117398 R0383:H08

SEQ ID NO:1198 is the determined cDNA sequence for clone 62117399 R0383:H09

SEQ ID NO:1199 is the determined cDNA sequence for clone 62117400 R0383:H10

SEQ ID NO:1200 is the determined cDNA sequence for clone 62117401 R0383:H11

SEQ ID NO:1201 is the determined cDNA sequence for clone 62177202 R0384:A02

SEQ ID NO:1202 is the determined cDNA sequence for clone 62177203 R0384:A03

SEQ ID NO:1203 is the determined cDNA sequence for clone 62177204 R0384:A05

SEQ ID NO:1204 is the determined cDNA sequence for clone 62177205 R0384:A06

SEQ ID NO:1205 is the determined cDNA sequence for clone 62177206 R0384:A07

SEQ ID NO:1206 is the determined cDNA sequence for clone 62177207 R0384:A08

SEQ ID NO:1207 is the determined cDNA sequence for clone 62177208 R0384:A09

SEQ ID NO:1208 is the determined cDNA sequence for clone 62177209 R0384:A10

SEQ ID NO:1209 is the determined cDNA sequence for clone 62177210 R0384:A11

SEQ ID NO:1210 is the determined cDNA sequence for clone 62177211 R0384:A12

SEQ ID NO:1211 is the determined cDNA sequence for clone 62177212 R0384:B01

SEQ ID NO:1212 is the determined cDNA sequence for clone 62177213 R0384:B02

SEQ ID NO:1213 is the determined cDNA sequence for clone 62177214 R0384:B03

SEQ ID NO:1214 is the determined cDNA sequence for clone 62177215 R0384:B04

SEQ ID NO:1215 is the determined cDNA sequence for clone 62177216 R0384:B05

SEQ ID NO:1216 is the determined cDNA sequence for clone 62177217 R0384:B06

SEQ ID NO:1217 is the determined cDNA sequence for clone 62177218 R0384:B07

SEQ ID NO:1218 is the determined cDNA sequence for clone 62177219 R0384:B08

SEQ ID NO:1219 is the determined cDNA sequence for clone 62177220 R0384:B09

SEQ ID NO:1220 is the determined cDNA sequence for clone 62177221 R0384:B10

SEQ ID NO:1221 is the determined cDNA sequence for clone 62177222 R0384:B11

SEQ ID NO:1222 is the determined cDNA sequence for clone 62177223 R0384:B12

SEQ ID NO:1223 is the determined cDNA sequence for clone 62177224 R0384:C01

SEQ ID NO:1224 is the determined cDNA sequence for clone 62177225 R0384:C02

SEQ ID NO:1225 is the determined cDNA sequence for clone 62177226 R0384:C03

SEQ ID NO:1226 is the determined cDNA sequence for clone 62177227 R0384:C04

SEQ ID NO:1227 is the determined cDNA sequence for clone 62177228 R0384:C05

SEQ ID NO:1228 is the determined cDNA sequence for clone 62177229 R0384:C06

SEQ ID NO:1229 is the determined cDNA sequence for clone 62177230 R0384:C07

SEQ ID NO:1230 is the determined cDNA sequence for clone 62177231 R0384:C08

SEQ ID NO:1231 is the determined cDNA sequence for clone 62177232 R0384:C09

SEQ ID NO:1232 is the determined cDNA sequence for clone 62177233 R0384:C10

SEQ ID NO:1233 is the determined cDNA sequence for clone 62177234 R0384:C11

SEQ ID NO:1234 is the determined cDNA sequence for clone 62177235 R0384:C12

SEQ ID NO:1235 is the determined cDNA sequence for clone 62177236 R0384:D01

SEQ ID NO:1236 is the determined cDNA sequence for clone 62177237 R0384:D02

SEQ ID NO:1237 is the determined cDNA sequence for clone 62177238 R0384:D03

SEQ ID NO:1238 is the determined cDNA sequence for clone 62177239 R0384:D04

SEQ ID NO:1239 is the determined cDNA sequence for clone 62177240 R0384:D05

SEQ ID NO:1240 is the determined cDNA sequence for clone 62177241 R0384:D06

SEQ ID NO:1241 is the determined cDNA sequence for clone 62177242 R0384:D07

SEQ ID NO:1242 is the determined cDNA sequence for clone 62177243 R0384:D08

SEQ ID NO:1243 is the determined cDNA sequence for clone 62177244 R0384:D09

SEQ ID NO:1244 is the determined cDNA sequence for clone 62177245 R0384:D10

SEQ ID NO:1245 is the determined cDNA sequence for clone 62177246 R0384:D11

SEQ ID NO:1246 is the determined cDNA sequence for clone 62177247 R0384:D12

SEQ ID NO:1247 is the determined cDNA sequence for clone 62177248 R0384:E01

SEQ ID NO:1248 is the determined cDNA sequence for clone 62177249 R0384:E02

SEQ ID NO:1249 is the determined cDNA sequence for clone 62177250 R0384:E03

SEQ ID NO:1250 is the determined cDNA sequence for clone 62177251 R0384:E04

SEQ ID NO:1251 is the determined cDNA sequence for clone 62177252 R0384:E05

SEQ ID NO:1252 is the determined cDNA sequence for clone 62177253 R0384:E06

SEQ ID NO:1253 is the determined cDNA sequence for clone 62177254 R0384:E07

SEQ ID NO:1254 is the determined cDNA sequence for clone 62177255 R0384:E08

SEQ ID NO:1255 is the determined cDNA sequence for clone 62177256 R0384:E09

SEQ ID NO:1256 is the determined cDNA sequence for clone 62177257 R0384:E06

SEQ ID NO:1257 is the determined cDNA sequence for clone 62177258 R0384:E11

SEQ ID NO:1258 is the determined cDNA sequence for clone 62177260 R0384:F01

SEQ ID NO:1259 is the determined cDNA sequence for clone 62177261 R0384:F02

SEQ ID NO:1260 is the determined cDNA sequence for clone 62177262 R0384:F03

SEQ ID NO:1261 is the determined cDNA sequence for clone 62177263 R0384:F04

SEQ ID NO:1262 is the determined cDNA sequence for clone 62177264 R0384:F05

SEQ ID NO:1263 is the determined cDNA sequence for clone 62177266 R0384:F07

SEQ ID NO:1264 is the determined cDNA sequence for clone 62177267 R0384:F08

SEQ ID NO:1265 is the determined cDNA sequence for clone 62177268 R0384:F09

SEQ ID NO:1266 is the determined cDNA sequence for clone 62177269 R0384:F10

SEQ ID NO:1267 is the determined cDNA sequence for clone 62177270 R0384:F11

SEQ ID NO:1268 is the determined cDNA sequence for clone 62177271 R0384:F12

SEQ ID NO:1269 is the determined cDNA sequence for clone 62177272 R0384:G01

SEQ ID NO:1270 is the determined cDNA sequence for clone 62177273 R0384:G02

SEQ ID NO:1271 is the determined cDNA sequence for clone 62177274 R0384:G03

SEQ ID NO:1272 is the determined cDNA sequence for clone 62177275 R0384:G04

SEQ ID NO:1273 is the determined cDNA sequence for clone 62177276 R0384:G05

SEQ ID NO:1274 is the determined cDNA sequence for clone 62177277 R0384:G06

SEQ ID NO:1275 is the determined cDNA sequence for clone 62177278 R0384:G07

SEQ ID NO:1276 is the determined cDNA sequence for clone 62177279 R0384:G08

SEQ ID NO:1277 is the determined cDNA sequence for clone 62177281 R0384:G10

SEQ ID NO:1278 is the determined cDNA sequence for clone 62177282 R0384:G11

SEQ ID NO:1279 is the determined cDNA sequence for clone 62177283 R0384:G12

SEQ ID NO:1280 is the determined cDNA sequence for clone 62177284 R0384:H01

SEQ ID NO:1281 is the determined cDNA sequence for clone 62177285 R0384:H02

SEQ ID NO:1282 is the determined cDNA sequence for clone 62177287 R0384:H04

SEQ ID NO:1283 is the determined cDNA sequence for clone 62177289 R0384:H06

SEQ ID NO:1284 is the determined cDNA sequence for clone 62177290 R0384:H07

SEQ ID NO:1285 is the determined cDNA sequence for clone 62177291 R0384:H08

SEQ ID NO:1286 is the determined cDNA sequence for clone 62177292 R0384:H09

SEQ ID NO:1287 is the determined cDNA sequence for clone 62177293 R0384:H10

SEQ ID NO:1288 is the determined cDNA sequence for clone 62177294 R0384:H11

SEQ ID NO:1289 is the determined cDNA sequence for clone 62117030 R0385:A02

SEQ ID NO:1290 is the determined cDNA sequence for clone 62117031 R0385:A03

SEQ ID NO:1291 is the determined cDNA sequence for clone 62117032 R0385:A05

SEQ ID NO:1292 is the determined cDNA sequence for clone 62117033 R0385:A06

SEQ ID NO:1293 is the determined cDNA sequence for clone 62117034 R03845:A07

SEQ ID NO:1294 is the determined cDNA sequence for clone 62117035 R0385:A08

SEQ ID NO:1295 is the determined cDNA sequence for clone 62117036 R0385:A09

SEQ ID NO:1296 is the determined cDNA sequence for clone 62117037 R0385:A10

SEQ ID NO:1297 is the determined cDNA sequence for clone 62117038 R0385:A11

SEQ ID NO:1298 is the determined cDNA sequence for clone 62117039 R0385:A12

SEQ ID NO:1299 is the determined cDNA sequence for clone 62117040 R0385:B01

SEQ ID NO:1300 is the determined cDNA sequence for clone 62117041 R0385:B02

SEQ ID NO:1301 is the determined cDNA sequence for clone 62117043 R0385 :B04

SEQ ID NO:1302 is the determined cDNA sequence for clone 62117044 R0385:B05

SEQ ID NO:1303 is the determined cDNA sequence for clone 62117045 R0385:B06

SEQ ID NO:1304 is the determined cDNA sequence for clone 62117046 R0385:B07

SEQ ID NO:1305 is the determined cDNA sequence for clone 62117047 R0385:B08

SEQ ID NO:1306 is the determined cDNA sequence for clone 62117048 R0385:B09

SEQ ID NO:1307 is the determined cDNA sequence for clone 62117049 R0385:B10

SEQ ID NO:1308 is the determined cDNA sequence for clone 62117050 R0385:B11

SEQ ID NO:1309 is the determined cDNA sequence for clone 62117051 R0385:B12

SEQ ID NO:1310 is the determined cDNA sequence for clone 62117052 R0385:C01

SEQ ID NO:1311 is the determined cDNA sequence for clone 62117053 R0385:C02

SEQ ID NO:1312 is the determined cDNA sequence for clone 62117055 R0385:C04

SEQ ID NO:1313 is the determined cDNA sequence for clone 62117056 R0385 :C05

SEQ ID NO:1314 is the determined cDNA sequence for clone 62117057 R0385:C06

SEQ ID NO:1315 is the determined cDNA sequence for clone 62117058 R0385:C07

SEQ ID NO:1316 is the determined cDNA sequence for clone 62117059 R0385:C08

SEQ ID NO:1317 is the determined cDNA sequence for clone 62117060 R0385:C09

SEQ ID NO:1318 is the determined cDNA sequence for clone 62117061 R0385:C10

SEQ ID NO:1319 is the determined cDNA sequence for clone 62117062 R0385:C11

SEQ ID NO:1320 is the determined cDNA sequence for clone 62117063 R0385:C12

SEQ ID NO:1321 is the determined cDNA sequence for clone 62117065 R0385:D02

SEQ ID NO:1322 is the determined cDNA sequence for clone 62117066 R0385:D03

SEQ ID NO:1323 is the determined cDNA sequence for clone 62117067 R0385:D04

SEQ ID NO:1324 is the determined cDNA sequence for clone 62117068 R0385:D05

SEQ ID NO:1325 is the determined cDNA sequence for clone 62117069 R0385:D06

SEQ ID NO:1326 is the determined cDNA sequence for clone 62117070 R0385:D07

SEQ ID NO:1327 is the determined cDNA sequence for clone 62117071 R0385:D08

SEQ ID NO:1328 is the determined cDNA sequence for clone 62117072 R0385:D09

SEQ ID NO:1329 is the determined cDNA sequence for clone 62117073 R0385:D10

SEQ ID NO:1330 is the determined cDNA sequence for clone 62117075 R0385:D12

SEQ ID NO:1331 is the determined cDNA sequence for clone 62117076 R0385:E01

SEQ ID NO:1332 is the determined cDNA sequence for clone 62117077 R0385:E02

SEQ ID NO:1333 is the determined cDNA sequence for clone 62117078 R0385:E03

SEQ ID NO:1334 is the determined cDNA sequence for clone 62117079 R0385:E04

SEQ ID NO:1335 is the determined cDNA sequence for clone 62117080 R0385:E05

SEQ ID NO:1336 is the determined cDNA sequence for clone 62117081 R0385:E06

SEQ ID NO:1337 is the determined cDNA sequence for clone 62117082 R0385:E07

SEQ ID NO:1338 is the determined cDNA sequence for clone 62117083 R0385:E08

SEQ ID NO:1339 is the determined cDNA sequence for clone 62117084 R0385:E09

SEQ ID NO:1340 is the determined cDNA sequence for clone 62117085 R0385:E10

SEQ ID NO:1341 is the determined cDNA sequence for clone 62117086 R0385:E11

SEQ ID NO:1342 is the determined cDNA sequence for clone 62117088 R0385:F01

SEQ ID NO:1343 is the determined cDNA sequence for clone 62117089 R0385:F02

SEQ ID NO:1344 is the determined cDNA sequence for clone 62117090 R0385:F03

SEQ ID NO:1345 is the determined cDNA sequence for clone 62117091 R0385:F04

SEQ ID NO:1346 is the determined cDNA sequence for clone 62117092 R0385:F05

SEQ ID NO:1347 is the determined cDNA sequence for clone 62117094 R0385:F07

SEQ ID NO:1348 is the determined cDNA sequence for clone 62117095 R0385:F08

SEQ ID NO:1349 is the determined cDNA sequence for clone 62117096 R0385:F09

SEQ ID NO:1350 is the determined cDNA sequence for clone 62117098 R0385:F11

SEQ ID NO:1351 is the determined cDNA sequence for clone 62117100 R0385:G01

SEQ ID NO:1352 is the determined cDNA sequence for clone 62117101 R0385: G02

SEQ ID NO:1353 is the determined cDNA sequence for clone 62117102 R0385:G03

SEQ ID NO:1354 is the determined cDNA sequence for clone 62117103 R0385:G04

SEQ ID NO:1355 is the determined cDNA sequence for clone 62117104 R0385:G05

SEQ ID NO:1356 is the determined cDNA sequence for clone 62117105 R0385:G06

SEQ ID NO:1357 is the determined cDNA sequence for clone 62117106 R0385:G07

SEQ ID NO:1358 is the determined cDNA sequence for clone 62117107 R0385:G08

SEQ ID NO:1359 is the determined cDNA sequence for clone 62117108 R0385:G09

SEQ ID NO:1360 is the determined cDNA sequence for clone 62117109 R0385:G10

SEQ ID NO:1361 is the determined cDNA sequence for clone 62117110 R0385:G11

SEQ ID NO:1362 is the determined cDNA sequence for clone 62117111 R0385:G12

SEQ ID NO:1363 is the determined cDNA sequence for clone 62117112 R0385:H01

SEQ ID NO:1364 is the determined cDNA sequence for clone 62117113 R0385:H02

SEQ ID NO:1365 is the determined cDNA sequence for clone 62117114 R0385:H03

SEQ ID NO:1366 is the determined cDNA sequence for clone 62117115 R0385:H04

SEQ ID NO:1367 is the determined cDNA sequence for clone 62117116 R0385:H05

SEQ ID NO:1368 is the determined cDNA sequence for clone 62117117 R0385:H06

SEQ ID NO:1369 is the determined cDNA sequence for clone 62117119 R0385:H04

SEQ ID NO:1370 is the determined cDNA sequence for clone 62117120 R0385:H09

SEQ ID NO:1371 is the determined cDNA sequence for clone 62117122 R0385:H11

SEQ ID NO:1372 is the determined cDNA sequence for clone 62117495 R0386:A02

SEQ ID NO:1373 is the determined cDNA sequence for clone 62117496 R0386:A03

SEQ ID NO:1374 is the determined cDNA sequence for clone 62117497 R03856:A05

SEQ ID NO:1375 is the determined cDNA sequence for clone 62117498 R0386:A06

SEQ ID NO:1376 is the determined cDNA sequence for clone 62117499 R0386:A07

SEQ ID NO:1377 is the determined cDNA sequence for clone 62117500 R0386:A08

SEQ ID NO:1378 is the determined cDNA sequence for clone 62117501 R0386:A09

SEQ ID NO:1379 is the determined cDNA sequence for clone 62117502 R0386:A10

SEQ ID NO:1380 is the determined cDNA sequence for clone 62117503 R0386:A11

SEQ ID NO:1381 is the determined cDNA sequence for clone 62117504 R0386:A12

SEQ ID NO:1382 is the determined cDNA sequence for clone 62117506 R0386:B02

SEQ ID NO:1383 is the determined cDNA sequence for clone 62117507 R0386:B03

SEQ ID NO:1384 is the determined cDNA sequence for clone 62117508 R0386:B04

SEQ ID NO:1385 is the determined cDNA sequence for clone 62117510 R0386:B06

SEQ ID NO:1386 is the determined cDNA sequence for clone 62117511 R0386:B07

SEQ ID NO:1387 is the determined cDNA sequence for clone 62117514 R0386:B10

SEQ ID NO:1388 is the determined cDNA sequence for clone 62117515 R0386:B11

SEQ ID NO:1389 is the determined cDNA sequence for clone 62117516 R0386:B12

SEQ ID NO:1390 is the determined cDNA sequence for clone 62117517 R0386:C01

SEQ ID NO:1391 is the determined cDNA sequence for clone 62117518 R0386:C02

SEQ ID NO:1392 is the determined cDNA sequence for clone 62117519 R0386:C03

SEQ ID NO:1393 is the determined cDNA sequence for clone 62117520 R0386:C04

SEQ ID NO:1394 is the determined cDNA sequence for clone 62117521 R0386:C05

SEQ ID NO:1395 is the determined cDNA sequence for clone 62117522 R0386:C06

SEQ ID NO:1396 is the determined cDNA sequence for clone 62117523 R0386:C07

SEQ ID NO:1397 is the determined cDNA sequence for clone 62117524 R0386:C08

SEQ ID NO:1398 is the determined cDNA sequence for clone 62117525 R0386:C09

SEQ ID NO:1399 is the determined cDNA sequence for clone 62117526 R0386:C10

SEQ ID NO:1400 is the determined cDNA sequence for clone 62117527 R0386:C11

SEQ ID NO:1401 is the determined cDNA sequence for clone 62117528 R0386:C12

SEQ ID NO:1402 is the determined cDNA sequence for clone 62117529 R0386:D01

SEQ ID NO:1403 is the determined cDNA sequence for clone 62117530 R0386:D02

SEQ ID NO:1404 is the determined cDNA sequence for clone 62117531 R0386:D03

SEQ ID NO:1405 is the determined cDNA sequence for clone 62117533 R0386:D05

SEQ ID NO:1406 is the determined cDNA sequence for clone 62117534 R0386:D06

SEQ ID NO:1407 is the determined cDNA sequence for clone 62117535 R0386:D07

SEQ ID NO:1408 is the determined cDNA sequence for clone 62117536 R0386:D02

SEQ ID NO:1409 is the determined cDNA sequence for clone 62117537 R0386:D09

SEQ ID NO:1410 is the determined cDNA sequence for clone 62117538 R0386:D10

SEQ ID NO:1411 is the determined cDNA sequence for clone 62117539 R0386:D11

SEQ ID NO:1412 is the determined cDNA sequence for clone 62117540 R0386:D12

SEQ ID NO:1413 is the determined cDNA sequence for clone 62117541 R0386:E01

SEQ ID NO:1414 is the determined cDNA sequence for clone 62117542 R0386:E02

SEQ ID NO:1415 is the determined cDNA sequence for clone 62117545 R0386:E05

SEQ ID NO:1416 is the determined cDNA sequence for clone 62117546 R0386:E06

SEQ ID NO:1417 is the determined cDNA sequence for clone 62117547 R0386:E07

SEQ ID NO:1418 is the determined cDNA sequence for clone 62117548 R0386:E08

SEQ ID NO:1419 is the determined cDNA sequence for clone 62117549 R0386:E09

SEQ ID NO:1420 is the determined cDNA sequence for clone 62117550 R0386:E10

SEQ ID NO:1421 is the determined cDNA sequence for clone 62117551 R0386:E11

SEQ ID NO:1422 is the determined cDNA sequence for clone 62117552 R0386:E12

SEQ ID NO:1423 is the determined cDNA sequence for clone 62117553 R0386:F01

SEQ ID NO:1424 is the determined cDNA sequence for clone 62117554 R0386:F02

SEQ ID NO:1425 is the determined cDNA sequence for clone 62117556 R0386:F04

SEQ ID NO:1426 is the determined cDNA sequence for clone 62117557 R0386:F05

SEQ ID NO:1427 is the determined cDNA sequence for clone 62117558 R0386:F06

SEQ ID NO:1428 is the determined cDNA sequence for clone 62117559 R0386:F07

SEQ ID NO:1429 is the determined cDNA sequence for clone 62117560 R0386:F08

SEQ ID NO:1430 is the determined cDNA sequence for clone 62117561 R0386:F09

SEQ ID NO:1431 is the determined cDNA sequence for clone 62117563 R0386:F11

SEQ ID NO:1432 is the determined cDNA sequence for clone 62117564 R0386:F12

SEQ ID NO:1433 is the determined cDNA sequence for clone 62117566 R0386:G02

SEQ ID NO:1434 is the determined cDNA sequence for clone 62117567 R0386:G03

SEQ ID NO:1435 is the determined cDNA sequence for clone 62117568 R0386:G04

SEQ ID NO:1436 is the determined cDNA sequence for clone 62117569 R0386:F05

SEQ ID NO:1437 is the determined cDNA sequence for clone 62117570 R0386:G06

SEQ ID NO:1438 is the determined cDNA sequence for clone 62117571 R0386:G07

SEQ ID NO:1439 is the determined cDNA sequence for clone 62117572 R0386:G08

SEQ ID NO:1440 is the determined cDNA sequence for clone 62117573 R0386:G09

SEQ ID NO:1441 is the determined cDNA sequence for clone 62117574 R0386:G10

SEQ ID NO:1442 is the determined cDNA sequence for clone 62117575 R0386:G11

SEQ ID NO:1443 is the determined cDNA sequence for clone 62117576 R0386:G12

SEQ ID NO:1444 is the determined cDNA sequence for clone 62117579 R0386:H03

SEQ ID NO:1445 is the determined cDNA sequence for clone 62117580 R0386:H04

SEQ ID NO:1446 is the determined cDNA sequence for clone 62117581 R0386:H05

SEQ ID NO:1447 is the determined cDNA sequence for clone 62117582 R0386:H06

SEQ ID NO:1448 is the determined cDNA sequence for clone 62117583 R0386:H07

SEQ ID NO:1449 is the determined cDNA sequence for clone 62117584 R0386:H08

SEQ ID NO:1450 is the determined cDNA sequence for clone 62117585 R0386:H09

SEQ ID NO:1451 is the determined cDNA sequence for clone 62117586 R0386:H10

SEQ ID NO:1452 is the determined cDNA sequence for clone 62117587 R0386:H11

SEQ ID NO:1453 is the determined cDNA sequence for clone 62227248 R0387:A05

SEQ ID NO:1454 is the determined cDNA sequence for clone 62227249 R0387:A06

SEQ ID NO:1455 is the determined cDNA sequence for clone 62227250 R0387:A07

SEQ ID NO:1456 is the determined cDNA sequence for clone 62227251 R0387:A8

SEQ ID NO:1457 is the determined cDNA sequence for clone 62227252 R0387:A09

SEQ ID NO:1458 is the determined cDNA sequence for clone 62227253 R0387:A10

SEQ ID NO:1459 is the determined cDNA sequence for clone 62227254 R0387:A11

SEQ ID NO:1460 is the determined cDNA sequence for clone 62227255 R0387:A12

SEQ ID NO:1461 is the determined cDNA sequence for clone 62227257 R0387:B02

SEQ ID NO:1462 is the determined cDNA sequence for clone 62227258 R0387:B03

SEQ ID NO:1463 is the determined cDNA sequence for clone 62227259 R0387:B04

SEQ ID NO:1464 is the determined cDNA sequence for clone 62227260 R0387:B05

SEQ ID NO:1465 is the determined cDNA sequence for clone 62227261 R0387:B06

SEQ ID NO:1466 is the determined cDNA sequence for clone 62227262 R0387:B07

SEQ ID NO:1467 is the determined cDNA sequence for clone 62227263 R0387:B08

SEQ ID NO:1468 is the determined cDNA sequence for clone 62227264 R0387:B09

SEQ ID NO:1469 is the determined cDNA sequence for clone 62227265 R0387:B10

SEQ ID NO:1470 is the determined cDNA sequence for clone 62227266 R0387:B11

SEQ ID NO:1471 is the determined cDNA sequence for clone 62227267 R0387:B 12

SEQ ID NO:1472 is the determined cDNA sequence for clone 62227268 R0387:C01

SEQ ID NO:1473 is the determined cDNA sequence for clone 62227269 R0387:C02

SEQ ID NO:1474 is the determined cDNA sequence for clone 62227270 R0387:C03

SEQ ID NO:1475 is the determined cDNA sequence for clone 62227271 R0387:C04

SEQ ID NO:1476 is the determined cDNA sequence for clone 62227272 R0387:C05

SEQ ID NO:1477 is the determined cDNA sequence for clone 62227274 R0387:C07

SEQ ID NO:1478 is the determined cDNA sequence for clone 62227275 R0387:C08

SEQ ID NO:1479 is the determined cDNA sequence for clone 62227276 R0387:C09

SEQ ID NO:1480 is the determined cDNA sequence for clone 62227277 R0387:C10

SEQ ID NO:1481 is the determined cDNA sequence for clone 62227278 R0387:C11

SEQ ID NO:1482 is the determined cDNA sequence for clone 62227279 R0387:C12

SEQ ID NO:1483 is the determined cDNA sequence for clone 62227280 R0387:D01

SEQ ID NO:1484 is the determined cDNA sequence for clone 62227281 R0387:D02

SEQ ID NO:1485 is the determined cDNA sequence for clone 62227283 R0387:D04

SEQ ID NO:1486 is the determined cDNA sequence for clone 62227285 R0387:D06

SEQ ID NO:1487 is the determined cDNA sequence for clone 62227286 R0387:D07

SEQ ID NO:1488 is the determined cDNA sequence for clone 62227289 R0387:D10

SEQ ID NO:1489 is the determined cDNA sequence for clone 62227290 R0387:D11

SEQ ID NO:1490 is the determined cDNA sequence for clone 62227291 R0387:D12

SEQ ID NO:1491 is the determined cDNA sequence for clone 62227294 R0387:E03

SEQ ID NO:1492 is the determined cDNA sequence for clone 62227295 R0387:E04

SEQ ID NO:1493 is the determined cDNA sequence for clone 62227296 R0387:E05

SEQ ID NO:1494 is the determined cDNA sequence for clone 62227298 R0387:E07

SEQ ID NO:1495 is the determined cDNA sequence for clone 62227299 R0387:E08

SEQ ID NO:1496 is the determined cDNA sequence for clone 62227300 R0387:E09

SEQ ID NO:1497 is the determined cDNA sequence for clone 62227301 R0387:E10

SEQ ID NO:1498 is the determined cDNA sequence for clone 6222732 R0387:E11

SEQ ID NO:1499 is the determined cDNA sequence for clone 62227304 R0387:F01

SEQ ID NO:1500 is the determined cDNA sequence for clone 62227305 R0387:F02

SEQ ID NO:1501 is the determined cDNA sequence for clone 62227307 R0387:F04

SEQ ID NO:1502 is the determined cDNA sequence for clone 62227308 R0387:F05

SEQ ID NO:1503 is the determined cDNA sequence for clone 62227309 R0387:F06

SEQ ID NO:1504 is the determined cDNA sequence for clone 62227310 R0387:F07

SEQ ID NO:1505 is the determined cDNA sequence for clone 62227311 R0387:F08

SEQ ID NO:1506 is the determined cDNA sequence for clone 62227312 R0387:F09

SEQ ID NO:1507 is the determined cDNA sequence for clone 62227313 R0387:F10

SEQ ID NO:1508 is the determined cDNA sequence for clone 62227314 R0387:F11

SEQ ID NO:1509 is the determined cDNA sequence for clone 62227315 R0387:F12

SEQ ID NO:1510 is the determined cDNA sequence for clone 62227316 R0387:G01

SEQ ID NO:1511 is the determined cDNA sequence for clone 62227317 R0387:G02

SEQ ID NO:1512 is the determined cDNA sequence for clone 62227320 R0387:G05

SEQ ID NO:1513 is the determined cDNA sequence for clone 62227321 R0387:G06

SEQ ID NO:1514 is the determined cDNA sequence for clone 62227323 R0387:G08

SEQ ID NO:1515 is the determined cDNA sequence for clone 62227324 R0387:G09

SEQ ID NO:1516 is the determined cDNA sequence for clone 62227326 R0387:G11

SEQ ID NO:1517 is the determined cDNA sequence for clone 62227327 R0387:G12

SEQ ID NO:1518 is the determined cDNA sequence for clone 62227328 R0387:H01

SEQ ID NO:1519 is the determined cDNA sequence for clone 62227329 R0387:H02

SEQ ID NO:1520 is the determined cDNA sequence for clone 62227331 R0387:H04

SEQ ID NO:1521 is the determined cDNA sequence for clone 62227332 R0387:H05

SEQ ID NO:1522 is the determined cDNA sequence for clone 62227333 R0387:H06

SEQ ID NO:1523 is the determined cDNA sequence for clone 62227334 R0387:H07

SEQ ID NO:1524 is the determined cDNA sequence for clone 62227335 R0387:H08

SEQ ID NO:1525 is the determined cDNA sequence for clone 62227336 R0387:H09

SEQ ID NO:1526 is the determined cDNA sequence for clone 62227338 R0387:H11

SEQ ID NO:1527 is the determined cDNA sequence for clone 62226968 R0387:A03

SEQ ID NO:1528 is the determined cDNA sequence for clone 62226969 R0388:A05

SEQ ID NO:1529 is the determined cDNA sequence for clone 62226970 R0388:A06

SEQ ID NO:1530 is the determined cDNA sequence for clone 62226971 R0388:A07

SEQ ID NO:1531 is the determined cDNA sequence for clone 62226972 R0388:A08

SEQ ID NO:1532 is the determined cDNA sequence for clone 62226973 R0388:A09

SEQ ID NO:1533 is the determined cDNA sequence for clone 62226974 R0388:A10

SEQ ID NO:1534 is the determined cDNA sequence for clone 62226975 R0388:A11

SEQ ID NO:1535 is the determined cDNA sequence for clone 62226976 R0388:A12

SEQ ID NO:1536 is the determined cDNA sequence for clone 62226977 R0388:B01

SEQ ID NO:1537 is the determined cDNA sequence for clone 62226978 R0388:B02

SEQ ID NO:1538 is the determined cDNA sequence for clone 62226980 R0388:B04

SEQ ID NO:1539 is the determined cDNA sequence for clone 62226982 R0388:B06

SEQ ID NO:1540 is the determined cDNA sequence for clone 62226984 R0388:B08

SEQ ID NO:1541 is the determined cDNA sequence for clone 62226985 R0388:B09

SEQ ID NO:1542 is the determined cDNA sequence for clone 62226986 R0388:B10

SEQ ID NO:1543 is the determined cDNA sequence for clone 62226987 R0388:B11

SEQ ID NO:1544 is the determined cDNA sequence for clone 62226988 R0388:B12

SEQ ID NO:1545 is the determined cDNA sequence for clone 62226989 R0388: C01

SEQ ID NO:1546 is the determined cDNA sequence for clone 62226990 R0388:C02

SEQ ID NO:1547 is the determined cDNA sequence for clone 62226991 R0388: C03

SEQ ID NO:1548 is the determined cDNA sequence for clone 62226992 R0388:C04

SEQ ID NO:1549 is the determined cDNA sequence for clone 62226993 R0388:C05

SEQ ID NO:1550 is the determined cDNA sequence for clone 62226994 R0388:C06

SEQ ID NO:1551 is the determined cDNA sequence for clone 62226995 R0388:C07

SEQ ID NO:1552 is the determined cDNA sequence for clone 62226996 R0388:C08

SEQ ID NO:1553 is the determined cDNA sequence for clone 62226997 R0388:C09

SEQ ID NO:1554 is the determined cDNA sequence for clone 62226998 R0388:C10

SEQ ID NO:1555 is the determined cDNA sequence for clone 62226999 R0388:C11

SEQ ID NO:1556 is the determined cDNA sequence for clone 62227000 R0388:C12

SEQ ID NO:1557 is the determined cDNA sequence for clone 62227001 R0388:D01

SEQ ID NO:1558 is the determined cDNA sequence for clone 62227003 R0388:D03

SEQ ID NO:1559 is the determined cDNA sequence for clone 62227004 R0388:D04

SEQ ID NO:1560 is the determined cDNA sequence for clone 62227005 R0388:D05

SEQ ID NO:1561 is the determined cDNA sequence for clone 62227006 R0388:D06

SEQ ID NO:1562 is the determined cDNA sequence for clone 62227007 R0388:D07

SEQ ID NO:1563 is the determined cDNA sequence for clone 62227008 R0388:D08

SEQ ID NO:1564 is the determined cDNA sequence for clone 62227009 R0388:D09

SEQ ID NO:1565 is the determined cDNA sequence for clone 62227011 R0388:D11

SEQ ID NO:1566 is the determined cDNA sequence for clone 62227012 R0388:D12

SEQ ID NO:1567 is the determined cDNA sequence for clone 62227013 R0388:E01

SEQ ID NO:1568 is the determined cDNA sequence for clone 62227015 R0388:E03

SEQ ID NO:1569 is the determined cDNA sequence for clone 62227018 R0388:E06

SEQ ID NO:1570 is the determined cDNA sequence for clone 62227019 R0388:E:07

SEQ ID NO:1571 is the determined cDNA sequence for clone 62227020 R0388:E08

SEQ ID NO:1572 is the determined cDNA sequence for clone 62227022 R0388:E10

SEQ ID NO:1573 is the determined cDNA sequence for clone 62227023 R0388:E11

SEQ ID NO:1574 is the determined cDNA sequence for clone 62227024 R0388:E12

SEQ ID NO:1575 is the determined cDNA sequence for clone 62227029 R0388:F:05

SEQ ID NO:1576 is the determined cDNA sequence for clone 62227030 R0388:F06

SEQ ID NO:1577 is the determined cDNA sequence for clone 62227031 R0388.:F07

SEQ ID NO:1578 is the determined cDNA sequence for clone 62227032 R0388:F08

SEQ ID NO:1579 is the determined cDNA sequence for clone 62227033 R0388:F09

SEQ ID NO:1580 is the determined cDNA sequence for clone 62227034 R0388:F10

SEQ ID NO:1581 is the determined cDNA sequence for clone 62227035 R0388:F11

SEQ ID NO:1582 is the determined cDNA sequence for clone 62227036 R0388:F12

SEQ ID NO:1583 is the determined cDNA sequence for clone 62227037 R0388:G01

SEQ ID NO:1584 is the determined cDNA sequence for clone 62227038 R0388:G02

SEQ ID NO:1585 is the determined cDNA sequence for clone 62227040 R0388: G04

SEQ ID NO:1586 is the determined cDNA sequence for clone 62227042 R0388:G06

SEQ ID NO:1587 is the determined cDNA sequence for clone 62227044 R0388:G08

SEQ ID NO:1588 is the determined cDNA sequence for clone 62227045 R0388:G09

SEQ ID NO:1589 is the determined cDNA sequence for clone 62227047 R0388:G11

SEQ ID NO:1590 is the determined cDNA sequence for clone 62227048 R0388:G12

SEQ ID NO:1591 is the determined cDNA sequence for clone 62227049 R0388:H01

SEQ ID NO:1592 is the determined cDNA sequence for clone 62227051 R0388:H03

SEQ ID NO:1593 is the determined cDNA sequence for clone 62227052 R0388:H04

SEQ ID NO:1594 is the determined cDNA sequence for clone 62227053 R0388:H05

SEQ ID NO:1595 is the determined cDNA sequence for clone 62227054 R0388:H06

SEQ ID NO:1596 is the determined cDNA sequence for clone 62227055 R0388:H07

SEQ ID NO:1597 is the determined cDNA sequence for clone 62227056 R0388:H08

SEQ ID NO:1598 is the determined cDNA sequence for clone 62227057 R0388:H09

SEQ ID NO:1599 is the determined cDNA sequence for clone 62227058 R0388:H10

SEQ ID NO:1600 is the determined cDNA sequence for clone 62227059 R0388:H11

SEQ ID NO:1601 is the determined cDNA sequence for clone 62227432 R0389:A02

SEQ ID NO:1602 is the determined cDNA sequence for clone 62227433 R0389:A03

SEQ ID NO:1603 is the determined cDNA sequence for clone 62227437 R0389:A08

SEQ ID NO:1604 is the determined cDNA sequence for clone 62227438 R0389:A09

SEQ ID NO:1605 is the determined cDNA sequence for clone 62227439 R0389:A10

SEQ ID NO:1606 is the determined cDNA sequence for clone 62227440 R0389:A11

SEQ ID NO:1607 is the determined cDNA sequence for clone 62227441 R0389:A12

SEQ ID NO:1608 is the determined cDNA sequence for clone 62227442 R0389:B01

SEQ ID NO:1609 is the determined cDNA sequence for clone 62227443 R0389:B02

SEQ ID NO:1610 is the determined cDNA sequence for clone 62227444 R0389:B03

SEQ ID NO:1611 is the determined cDNA sequence for clone 62227445 R0389:B04

SEQ ID NO:1612 is the determined cDNA sequence for clone 62227446 R0389:B05

SEQ ID NO:1613 is the determined cDNA sequence for clone 62227447 R0389:B06

SEQ ID NO:1614 is the determined cDNA sequence for clone 62227448 R0389:B07

SEQ ID NO:1615 is the determined cDNA sequence for clone 62227450 R0389:B09

SEQ ID NO:1616 is the determined cDNA sequence for clone 62227451 R0389:B10

SEQ ID NO:1617 is the determined cDNA sequence for clone 62227452 R0389:B11

SEQ ID NO:1618 is the determined cDNA sequence for clone 62227453 R0389:B12

SEQ ID NO:1619 is the determined cDNA sequence for clone 62227456 R0389:C03

SEQ ID NO:1620 is the determined cDNA sequence for clone 62227457 R0389:C04

SEQ ID NO:1621 is the determined cDNA sequence for clone 62227458 R0389:C05

SEQ ID NO:1622 is the determined cDNA sequence for clone 62227459 R0389:C06

SEQ ID NO:1623 is the determined cDNA sequence for clone 62227460 R0389:C07

SEQ ID NO:1624 is the determined cDNA sequence for clone 62227461 R0389:C08

SEQ ID NO:1625 is the determined cDNA sequence for clone 62227462 R0389:C09

SEQ ID NO:1626 is the determined cDNA sequence for clone 62227463 R0389:C10

SEQ ID NO:1627 is the determined cDNA sequence for clone 62227464 R0389:C11

SEQ ID NO:1628 is the determined cDNA sequence for clone 62227465 R0389:C12

SEQ ID NO:1629 is the determined cDNA sequence for clone 62227466 R0389:D01

SEQ ID NO:1630 is the determined cDNA sequence for clone 62227468 R0389:D03

SEQ ID NO:1631 is the determined cDNA sequence for clone 62227469 R0389:D04

SEQ ID NO:1632 is the determined cDNA sequence for clone 62227470 R0389:D05

SEQ ID NO:1633 is the determined cDNA sequence for clone 62227471 R0389:D06

SEQ ID NO:1634 is the determined cDNA sequence for clone 62227472 R0389:D07

SEQ ID NO:1635 is the determined cDNA sequence for clone 62227473 R0389:D08

SEQ ID NO:1636 is the determined cDNA sequence for clone 62227475 R0389:D10

SEQ ID NO:1637 is the determined cDNA sequence for clone 62227476 R0389:D11

SEQ ID NO:1638 is the determined cDNA sequence for clone 62227477 R0389:D12

SEQ ID NO:1639 is the determined cDNA sequence for clone 62227478 R0389:E01

SEQ ID NO:1640 is the determined cDNA sequence for clone 62227479 R0389:E02

SEQ ID NO:1641 is the determined cDNA sequence for clone 62227480 R0389:E03

SEQ ID NO:1642 is the determined cDNA sequence for clone 62227481 R0389:E04

SEQ ID NO:1643 is the determined cDNA sequence for clone 62227482 R0389:E05

SEQ ID NO:1644 is the determined cDNA sequence for clone 62227483 R0389:E06

SEQ ID NO:1645 is the determined cDNA sequence for clone 62227484 R0389:E07

SEQ ID NO:1646 is the determined cDNA sequence for clone 62227485 R0389:E08

SEQ ID NO:1647 is the determined cDNA sequence for clone 62227487 R0389:E10

SEQ ID NO:1648 is the determined cDNA sequence for clone 62227488 R0389:E11

SEQ ID NO:1649 is the determined cDNA sequence for clone 62227490 R0389:F01

SEQ ID NO:1650 is the determined cDNA sequence for clone 62227491 R0389:F02

SEQ ID NO:1651 is the determined cDNA sequence for clone 62227492 R0389:F03

SEQ ID NO:1652 is the determined cDNA sequence for clone 62227493 R0389:F04

SEQ ID NO:1653 is the determined cDNA sequence for clone 62227495 R0389:F06

SEQ ID NO:1654 is the determined cDNA sequence for clone 62227496 R0389:F7

SEQ ID NO:1655 is the determined cDNA sequence for clone 62227497 R0389:F08

SEQ ID NO:1656 is the determined cDNA sequence for clone 62227498 R0389:F09

SEQ ID NO:1657 is the determined cDNA sequence for clone 62227499 R0389:F10

SEQ ID NO:1658 is the determined cDNA sequence for clone 62227500 R0389:F11

SEQ ID NO:1659 is the determined cDNA sequence for clone 62227501 R0389:F12

SEQ ID NO:1660 is the determined cDNA sequence for clone 62227502 R0389:G01

SEQ ID NO:1661 is the determined cDNA sequence for clone 62227505 R0389:G04

SEQ ID NO:1662 is the determined cDNA sequence for clone 62227507 R0389:G06

SEQ ID NO:1663 is the determined cDNA sequence for clone 62227508 R0389:G07

SEQ ID NO:1664 is the determined cDNA sequence for clone 62227511 R0389:G10

SEQ ID NO:1665 is the determined cDNA sequence for clone 62227512 R0389:G11

SEQ ID NO:1666 is the determined cDNA sequence for clone 62227515 R0389:H02

SEQ ID NO:1667 is the determined cDNA sequence for clone 62227516 R0389:H03

SEQ ID NO:1668 is the determined cDNA sequence for clone 62227517 R0389:H04

SEQ ID NO:1669 is the determined cDNA sequence for clone 62227519 R0389:H06

SEQ ID NO:1670 is the determined cDNA sequence for clone 62227520 R0389:H07

SEQ ID NO:1671 is the determined cDNA sequence for clone 62227521 R0389:H08

SEQ ID NO:1672 is the determined cDNA sequence for clone 62227522 R0389:H09

SEQ ID NO:1673 is the determined cDNA sequence for clone 62227523 R0389:H10

SEQ ID NO:1674 is the determined cDNA sequence for clone 62227524 R0389:H11

SEQ ID NO:1675 is the determined cDNA sequence for clone 62227526 R0390:A03

SEQ ID NO:1676 is the determined cDNA sequence for clone 62227527 R0390:A05

SEQ ID NO:1677 is the determined cDNA sequence for clone 62227528 R0390:A06

SEQ ID NO:1678 is the determined cDNA sequence for clone 62227529 R0390:A07

SEQ ID NO:1679 is the determined cDNA sequence for clone 62227530 R0390:A08

SEQ ID NO:1680 is the determined cDNA sequence for clone 62227532 R0390:A10

SEQ ID NO:1681 is the determined cDNA sequence for clone 62227533 R0390:A11

SEQ ID NO:1682 is the determined cDNA sequence for clone 62227535 R0390:B01

SEQ ID NO:1683 is the determined cDNA sequence for clone 62227536 R0390:B02

SEQ ID NO:1684 is the determined cDNA sequence for clone 62227537 R0390:B03

SEQ ID NO:1685 is the determined cDNA sequence for clone 62227538 R0390:B04

SEQ ID NO:1686 is the determined cDNA sequence for clone 62227541 R0390:B07

SEQ ID NO:1687 is the determined cDNA sequence for clone 62227542 R0390:B08

SEQ ID NO:1688 is the determined cDNA sequence for clone 62227543 R0390:B09

SEQ ID NO:1689 is the determined cDNA sequence for clone 62227544 R0390:B10

SEQ ID NO:1690 is the determined cDNA sequence for clone 62227545 R0390:B01

SEQ ID NO:1691 is the determined :DNA sequence for clone 62227546 R0390:B12

SEQ ID NO:1692 is the determined cDNA sequence for clone 62227547 R0390:C01

SEQ ID NO:1693 is the determined cDNA sequence for clone 62227548 R0390:C02

SEQ ID NO:1694 is the determined cDNA sequence for clone 62227549 R0390:C03

SEQ ID NO:1695 is the determined cDNA sequence for clone 62227550 R0390:C04

SEQ ID NO:1696 is the determined cDNA sequence for clone 62227551 R0390:C05

SEQ ID NO:1697 is the determined cDNA sequence for clone 62227552 R0390:C06

SEQ ID NO:1698 is the determined cDNA sequence for clone 62227553 R0390:C07

SEQ ID NO:1699 is the determined cDNA sequence for clone 62227554 R0390:C08

SEQ ID NO:1700 is the determined cDNA sequence for clone 62227555 R0390:C09

SEQ ID NO:1701 is the determined cDNA sequence for clone 62227556 R0390:C10

SEQ ID NO:1702 is the determined cDNA sequence for clone 62227557 R0390:C11

SEQ ID NO:1703 is the determined cDNA sequence for clone 62227558 R0390:C12

SEQ ID NO:1704 is the determined cDNA sequence for clone 62227559 R0390:D01

SEQ ID NO:1705 is the determined cDNA sequence for clone 62227560 R0390:D02

SEQ ID NO:1706 is the determined cDNA sequence for clone 62227562 R0390:D04

SEQ ID NO:1707 is the determined cDNA sequence for clone 62227563 R0390:D05

SEQ ID NO:1708 is the determined cDNA sequence for clone 62227564 R0390:D06

SEQ ID NO:1709 is the determined cDNA sequence for clone 62227565 R0390:D07

SEQ ID NO:1710 is the determined cDNA sequence for clone 62227566 R0390:D08

SEQ ID NO:1711 is the determined cDNA sequence for clone 62227569 R0390:D11

SEQ ID NO:1712 is the determined cDNA sequence for clone 62227570 R0390:D12

SEQ ID NO:1713 is the determined cDNA sequence for clone 62227571 R0390:E01

SEQ ID NO:1714 is the determined cDNA sequence for clone 62227572 R0390:E02

SEQ ID NO:1715 is the determined cDNA sequence for clone 62227573 R0390:E03

SEQ ID NO:1716 is the determined cDNA sequence for clone 62227575 R0390:E05

SEQ ID NO:1717 is the determined cDNA sequence for clone 62227576 R0390:E06

SEQ ID NO:1718 is the determined cDNA sequence for clone 62227577 R0390:E07

SEQ ID NO:1719 is the determined cDNA sequence for clone 62227578 R0390:E08

SEQ ID NO:1720 is the determined cDNA sequence for clone 62227581 R0390:E11

SEQ ID NO:1721 is the determined cDNA sequence for clone 62227583 R0390:F01

SEQ ID NO:1722 is the determined cDNA sequence for clone 62227584 R0390:F02

SEQ ID NO:1723 is the determined cDNA sequence for clone 62227585 R0390:F03

SEQ ID NO:1724 is the determined cDNA sequence for clone 62227586 R0390:F04

SEQ ID NO:1725 is the determined cDNA sequence for clone 62227589 R0390:F07

SEQ ID NO:1726 is the determined cDNA sequence for clone 62227590 R0390:F08

SEQ ID NO:1727 is the determined cDNA sequence for clone 62227591 R0390:F09

SEQ ID NO:1728 is the determined cDNA sequence for clone 62227593 R0390:F11

SEQ ID NO:1729 is the determined cDNA sequence for clone 62227594 R0390:F12

SEQ ID NO:1730 is the determined cDNA sequence for clone 62227596 R0390:G02

SEQ ID NO:1731 is the determined cDNA sequence for clone 62227598 R0390:G04

SEQ ID NO:1732 is the determined cDNA sequence for clone 62227599 R0390:G05

SEQ ID NO:1733 is the determined cDNA sequence for clone 62227600 R0390:G06

SEQ ID NO:1734 is the determined cDNA sequence for clone 62227601 R0390:G07

SEQ ID NO:1735 is the determined cDNA sequence for clone 62227602 R0390:G08

SEQ ID NO:1736 is the determined cDNA sequence for clone 62227603 R0390:G09

SEQ ID NO:1737 is the determined cDNA sequence for clone 62227605 R0390:G11

SEQ ID NO:1738 is the determined cDNA sequence for clone 62227607 R0390:H01

SEQ ID NO:1739 is the determined cDNA sequence for clone 62227608 R0390:H02

SEQ ID NO:1740 is the determined cDNA sequence for clone 62227609 R0390:H03

SEQ ID NO:1741 is the determined cDNA sequence for clone 62227610 R0390:H04

SEQ ID NO:1742 is the determined cDNA sequence for clone 62227611 R0390:H05

SEQ ID NO:1743 is the determined cDNA sequence for clone 62227613 R0390:H07

SEQ ID NO:1744 is the determined cDNA sequence for clone 62227614 R0390:H08

SEQ ID NO:1745 is the determined cDNA sequence for clone 62227616 R0390:H10

SEQ ID NO:1746 is the determined cDNA sequence for clone 62227617 R0390:H11

SEQ ID NO:1747 is the determined cDNA sequence for clone 62227339 R0391:A02

SEQ ID NO:1748 is the determined cDNA sequence for clone 62227340 R0391:A03

SEQ ID NO:1749 is the determined cDNA sequence for clone 62227341 R0391:A05

SEQ ID NO:1750 is the determined cDNA sequence for clone 62227342 R0391:A06

SEQ ID NO:1751 is the determined cDNA sequence for clone 62227343 R0391:A07

SEQ ID NO:1752 is the determined .DNA sequence for clone 62227344 R0391:A08

SEQ ID NO:1753 is the determined cDNA sequence for clone 62227345 R0391:A09

SEQ ID NO:1754 is the determined cDNA sequence for clone 62227346 R0391:A10

SEQ ID NO:1755 is the determined cDNA sequence for clone 62227348 R0391:A12

SEQ ID NO:1756 is the determined cDNA sequence for clone 62227349 R0391:B01

SEQ ID NO:1757 is the determined cDNA sequence for clone 62227352 R0391:B04

SEQ ID NO:1758 is the determined cDNA sequence for clone 62227353 R0391:B05

SEQ ID NO:1759 is the determined cDNA sequence for clone 62227355 R0391:B07

SEQ ID NO:1760 is the determined cDNA sequence for clone 62227356 R0391:B08

SEQ ID NO:1761 is the determined cDNA sequence for clone 62227359 R0391:B11

SEQ ID NO:1762 is the determined cDNA sequence for clone 62227360 R0391:B12

SEQ ID NO:1763 is the determined cDNA sequence for clone 62227361 R0391:C01

SEQ ID NO:1764 is the determined cDNA sequence for clone 62227362 R0391:C02

SEQ ID NO:1765 is the determined cDNA sequence for clone 62227363 R0391:C03

SEQ ID NO:1766 is the determined cDNA sequence for clone 62227364 R0391:C04

SEQ ID NO:1767 is the determined cDNA sequence for clone 62227365 R0391:C05

SEQ ID NO:1768 is the determined cDNA sequence for clone 62227366 R0391:C06

SEQ ID NO:1769 is the determined cDNA sequence for clone 62227367 R0391:C07

SEQ ID NO:1770 is the determined cDNA sequence for clone 62227368 R0391:C08

SEQ ID NO:1771 is the determined cDNA sequence for clone 62227369 R0391:C09

SEQ ID NO:1772 is the determined cDNA sequence for clone 62227370 R0391:C10

SEQ ID NO:1773 is the determined cDNA sequence for clone 62227371 R0391:C11

SEQ ID NO:1774 is the determined cDNA sequence for clone 62227372 R0391:C12

SEQ ID NO:1775 is the determined cDNA sequence for clone 62227373 R0391:D01

SEQ ID NO:1776 is the determined cDNA sequence for clone 62227375 R0391:D03

SEQ ID NO:1777 is the determined cDNA sequence for clone 62227376 R0391:D04

SEQ ID NO:1778 is the determined cDNA sequence for clone 62227377 R0391:D05

SEQ ID NO:1779 is the determined cDNA sequence for clone 62227380 R0391:D08

SEQ ID NO:1780 is the determined cDNA sequence for clone 62227381 R0391:D09

SEQ ID NO:1781 is the determined cDNA sequence for clone 62227382 R0391:D10

SEQ ID NO:1782 is the determined cDNA sequence for clone 62227383 R0391:D11

SEQ ID NO:1783 is the determined cDNA sequence for clone 62227384 R0391:D12

SEQ ID NO:1784 is the determined cDNA sequence for clone 62227385 R0391:E01

SEQ ID NO:1785 is the determined cDNA sequence for clone 62227386 R0391:E02

SEQ ID NO:1786 is the determined cDNA sequence for clone 62227387 R0391:E03

SEQ ID NO:1787 is the determined cDNA sequence for clone 62227388 R0391:E04

SEQ ID NO:1788 is the determined cDNA sequence for clone 62227389 R0391:E05

SEQ ID NO:1789 is the determined cDNA sequence for clone 62227390 R0391:E06

SEQ ID NO:1790 is the determined cDNA sequence for clone 62227391 R0391:E07

SEQ ID NO:1791 is the determined cDNA sequence for clone 62227392 R0391:E08

SEQ ID NO:1792 is the determined cDNA sequence for clone 62227393 R0391:E09

SEQ ID NO:1793 is the determined cDNA sequence for clone 62227395 R0391:E11

SEQ ID NO:1794 is the determined cDNA sequence for clone 62227396 R0391:E12

SEQ ID NO:1795 is the determined cDNA sequence for clone 62227397 R0391:F01

SEQ ID NO:1796 is the determined cDNA sequence for clone 62227399 R0391:F03

SEQ ID NO:1797 is the determined cDNA sequence for clone 62227400 R0391:F04

SEQ ID NO:1798 is the determined cDNA sequence for clone 62227402 R0391:F06

SEQ ID NO:1799 is the determined cDNA sequence for clone 62227403 R0391:F07

SEQ ID NO:1800 is the determined cDNA sequence for clone 62227405 R0391:F09

SEQ ID NO:1801 is the determined cDNA sequence for clone 62227406 R0391:F10

SEQ ID NO:1802 is the determined cDNA sequence for clone 62227408 R0391:F12

SEQ ID NO:1803 is the determined cDNA sequence for clone 62227409 R0391:G01

SEQ ID NO:1804 is the determined cDNA sequence for clone 62227412 R0391:G04

SEQ ID NO:1805 is the determined cDNA sequence for clone 62227415 R0391:G07

SEQ ID NO:1806 is the determined cDNA sequence for clone 62227417 R0391:G09

SEQ ID NO:1807 is the determined cDNA sequence for clone 62227418 R0391:G10

SEQ ID NO:1808 is the determined cDNA sequence for clone 62227419 R0391:G11

SEQ ID NO:1809 is the determined cDNA sequence for clone 62227420 R0391:G12

SEQ ID NO:1810 is the determined cDNA sequence for clone 62227421 R0391:H01

SEQ ID NO:1811 is the determined cDNA sequence for clone 62227423 R0391:H03

SEQ ID NO:1812 is the determined cDNA sequence for clone 62227429 R0391:H09

SEQ ID NO:1813 is the determined cDNA sequence for clone 62227430 R0391:H10

SEQ ID NO:1814 is the determined cDNA sequence for clone 62227431 R0391:H11

SEQ ID NO:1815 is the determined cDNA sequence for clone 62342810 R0392:A02

SEQ ID NO:1816 is the determined cDNA sequence for clone 62342811 R0392:A03

SEQ ID NO:1817 is the determined cDNA sequence for clone 62342812 R0392:A05

SEQ ID NO:1818 is the determined cDNA sequence for clone 62342813 R0392:A06

SEQ ID NO:1819 is the determined cDNA sequence for clone 62342814 R0392:A07

SEQ ID NO:1820 is the determined cDNA sequence for clone 62342815 R0392:A08

SEQ ID NO:1821 is the determined cDNA sequence for clone 62342816 R0392:A09

SEQ ID NO:1822 is the determined cDNA sequence for clone 62342817 R0392:A10

SEQ ID NO:1823 is the determined cDNA sequence for clone 62342818 R0392:A11

SEQ ID NO:1824 is the determined cDNA sequence for clone 62342819 R0392:A12

SEQ ID NO:1825 is the determined cDNA sequence for clone 62342821 R0392:B02

SEQ ID NO:1826 is the determined cDNA sequence for clone 62342822 R0392:B03

SEQ ID NO:1827 is the determined cDNA sequence for clone 62342823 R0392:B04

SEQ ID NO:1828 is the determined cDNA sequence for clone 62342824 R0392:B05

SEQ ID NO:1829 is the determined cDNA sequence for clone 62342825 R0392:B06

SEQ ID NO:1830 is the determined cDNA sequence for clone 62342826 R0392:B07

SEQ ID NO:1831 is the determined cDNA sequence for clone 62342827 R0392:B08

SEQ ID NO:1832 is the determined :DNA sequence for clone 62342828 R0392:B09

SEQ ID NO:1833 is the determined cDNA sequence for clone 62342829 R0392:B10

SEQ ID NO:1834 is the determined cDNA sequence for clone 62342830 R0392:B11

SEQ ID NO:1835 is the determined cDNA sequence for clone 62342831 R0392:B12

SEQ ID NO:1836 is the determined cDNA sequence for clone 62342832 R0392:C01

SEQ ID NO:1837 is the determined cDNA sequence for clone 62342833 R0392:C02

SEQ ID NO:1838 is the determined cDNA sequence for clone 62342834 R0392:C03

SEQ ID NO:1839 is the determined cDNA sequence for clone 62342835 R0392:C04

SEQ ID NO:1840 is the determined cDNA sequence for clone 62342836 R0392:C05

SEQ ID NO:1841 is the determined cDNA sequence for clone 62342837 R0392:C06

SEQ ID NO:1842 is the determined cDNA sequence for clone 62342838 R0392:C07

SEQ ID NO:1843 is the determined cDNA sequence for clone 62342839 R0392:C08

SEQ ID NO:1844 is the determined cDNA sequence for clone 62342840 R0392:C09

SEQ ID NO:1845 is the determined cDNA sequence for clone 62342841 R0392:C10

SEQ ID NO:1846 is the determined cDNA sequence for clone 62342842 R0392:C11

SEQ ID NO:1847 is the determined cDNA sequence for clone 62342843 R0392:C12

SEQ ID NO:1848 is the determined cDNA sequence for clone 62342844 R0392:D01

SEQ ID NO:1849 is the determined cDNA sequence for clone 62342846 R0392:D03

SEQ ID NO:1850 is the determined cDNA sequence for clone 62342847 R0392:D04

SEQ ID NO:1851 is the determined cDNA sequence for clone 62342848 R0392:D05

SEQ ID NO:1852 is the determined cDNA sequence for clone 62342849 R0392:D06

SEQ ID NO:1853 is the determined cDNA sequence for clone 62342850 R0392:D07

SEQ ID NO:1854 is the determined cDNA sequence for clone 62342851 R0392:D08

SEQ ID NO:1855 is the determined cDNA sequence for clone 62342852 R0392:D09

SEQ ID NO:1856 is the determined cDNA sequence for clone 62342854 R0392:D11

SEQ ID NO:1857 is the determined cDNA sequence for clone 62342857 R0392:E02

SEQ ID NO:1858 is the determined cDNA sequence for clone 62342858 R0392:E03

SEQ ID NO:1859 is the determined cDNA sequence for clone 62342859 R0392:E04

SEQ ID NO:1860 is the determined cDNA sequence for clone 62342860 R0392:E05

SEQ ID NO:1861 is the determined cDNA sequence for clone 62342861 R0392:E06

SEQ ID NO:1862 is the determined cDNA sequence for clone 62342862 R0392:E07

SEQ ID NO:1863 is the determined cDNA sequence for clone 62342863 R0392:E08

SEQ ID NO:1864 is the determined cDNA sequence for clone 62342864 R0392:E09

SEQ ID NO:1865 is the determined cDNA sequence for clone 62342865 R0392:E10

SEQ ID NO:1866 is the determined cDNA sequence for clone 62342866 R0392:E11

SEQ ID NO:1867 is the determined cDNA sequence for clone 62342872 R0392:F05

SEQ ID NO:1868 is the determined cDNA sequence for clone 62342873 R0392:F06

SEQ ID NO:1869 is the determined cDNA sequence for clone 62342874 R0392:F07

SEQ ID NO:1870 is the determined cDNA sequence for clone 62342875 R0392:F08

SEQ ID NO:1871 is the determined cDNA sequence for clone 62342879 R0392:F12

SEQ ID NO:1872 is the determined cDNA sequence for clone 62342880 R0392:G01

SEQ ID NO:1873 is the determined cDNA sequence for clone 62342881 R0392:G02

SEQ ID NO:1874 is the determined cDNA sequence for clone 62342882 R0392:G03

SEQ ID NO:1875 is the determined cDNA sequence for clone 62342883 R0392:G04

SEQ ID NO:1876 is the determined cDNA sequence for clone 62342885 R0392:G06

SEQ ID NO:1877 is the determined cDNA sequence for clone 62342886 R0392:G07

SEQ ID NO:1878 is the determined cDNA sequence for clone 62342887 R0392:G08

SEQ ID NO:1879 is the determined cDNA sequence for clone 62342888 R0392:G09

SEQ ID NO:1880 is the determined cDNA sequence for clone 62342889 R0392:G10

SEQ ID NO:1881 is the determined cDNA sequence for clone 62342890 R0392:G11

SEQ ID NO:1882 is the determined cDNA sequence for clone 62342891 R0392:G12

SEQ ID NO:1883 is the determined cDNA sequence for clone 62342892 R0392:H01

SEQ ID NO:1884 is the determined cDNA sequence for clone 62342893 R0392:H02

SEQ ID NO:1885 is the determined cDNA sequence for clone 62342894 R0392:H03

SEQ ID NO:1886 is the determined cDNA sequence for clone 62342895 R0392:H04

SEQ ID NO:1887 is the determined cDNA sequence for clone 62342896 R0392:H05

SEQ ID NO:1888 is the determined cDNA sequence for clone 62342897 R0392:H06

SEQ ID NO:1889 is the determined cDNA sequence for clone 62342898 R0392:H07

SEQ ID NO:1890 is the determined cDNA sequence for clone 62342899 R0392:H08

SEQ ID NO:1891 is the determined cDNA sequence for clone 62342900 R0392:H09

SEQ ID NO:1892 is the determined cDNA sequence for clone 62342901 R0392:H10

SEQ ID NO:1893 is the determined cDNA sequence for clone 62177424 R0393:A02

SEQ ID NO:1894 is the determined cDNA sequence for clone 62177425 R0393:A03

SEQ ID NO:1895 is the determined cDNA sequence for clone 62177426 R0393:A05

SEQ ID NO:1896 is the determined cDNA sequence for clone 62177427 R0393:A06

SEQ ID NO:1897 is the determined cDNA sequence for clone 62177428 R0393:A07

SEQ ID NO:1898 is the determined cDNA sequence for clone 62177429 R0393:A08

SEQ ID NO:1899 is the determined cDNA sequence for clone 62177430 R0393:A09

SEQ ID NO:1900 is the determined cDNA sequence for clone 62177431 R0393 :A10

SEQ ID NO:1901 is the determined cDNA sequence for clone 62177432 R0393:A11

SEQ ID NO:1902 is the determined cDNA sequence for clone 62177433 R0393:A12

SEQ ID NO:1903 is the determined cDNA sequence for clone 62177435 R0393:B02

SEQ ID NO:1904 is the determined cDNA sequence for clone 62177436 R0393:B03

SEQ ID NO:1905 is the determined cDNA sequence for clone 62177438 R0393:B05

SEQ ID NO:1906 is the determined cDNA sequence for clone 62177439 R0393:B06

SEQ ID NO:1907 is the determined cDNA sequence for clone 62177441 R0393:B08

SEQ ID NO:1908 is the determined cDNA sequence for clone 62177442 R0393:B09

SEQ ID NO:1909 is the determined cDNA sequence for clone 62177443 R0393:B10

SEQ ID NO:1910 is the determined cDNA sequence for clone 62177444 R0393:B11

SEQ ID NO:1911 is the determined cDNA sequence for clone 62177445 R0393:B12

SEQ ID NO:1912 is the determined cDNA sequence for clone 62177446 R0393:C01

SEQ ID NO:1913 is the determined cDNA sequence for clone 62177447 R0393:C02

SEQ ID NO:1914 is the determined cDNA sequence for clone 62177448 R0393:C03

SEQ ID NO:1915 is the determined cDNA sequence for clone 62177449 R0393:C04

SEQ ID NO:1916 is the determined cDNA sequence for clone 62177450 R0393:C05

SEQ ID NO:1917 is the determined cDNA sequence for clone 62177451 R0393:C06

SEQ ID NO:1918 is the determined cDNA sequence for clone 62177452 R0393:C07

SEQ ID NO:1919 is the determined cDNA sequence for clone 62177453 R0393:C08

SEQ ID NO:1920 is the determined cDNA sequence for clone 62177454 R0393 :C09

SEQ ID NO:1921 is the determined cDNA sequence for clone 62177455 R0393:C10

SEQ ID NO:1922 is the determined cDNA sequence for clone 62177457 R0393:C12

SEQ ID NO:1923 is the determined cDNA sequence for clone 62177458 R0393:D01

SEQ ID NO:1924 is the determined cDNA sequence for clone 62177459 R0393:D02

SEQ ID NO:1925 is the determined cDNA sequence for clone 62177460 R0393:D03

SEQ ID NO:1926 is the determined cDNA sequence for clone 62177461 R0393:D04

SEQ ID NO:1927 is the determined cDNA sequence for clone 62177462 R0393:D05

SEQ ID NO:1928 is the determined cDNA sequence for clone 62177463 R0393:D06

SEQ ID NO:1929 is the determined cDNA sequence for clone 62177464 R0393:D07

SEQ ID NO:1930 is the determined cDNA sequence for clone 62177465 R0393:D08

SEQ ID NO:1931 is the determined cDNA sequence for clone 62177466 R0393:D09

SEQ ID NO:1932 is the determined cDNA sequence for clone 62177467 R0393:D10

SEQ ID NO:1933 is the determined cDNA sequence for clone 62177469 R0393:D 12

SEQ ID NO:1934 is the determined cDNA sequence for clone 62177470 R0393:E01

SEQ ID NO:1935 is the determined cDNA sequence for clone 62177471 R0393:E02

SEQ ID NO:1936 is the determined cDNA sequence for clone 62177472 R0393:E03

SEQ ID NO:1937 is the determined cDNA sequence for clone 62177473 R0393:E04

SEQ ID NO:1938 is the determined cDNA sequence for clone 62177474 R0393:E05

SEQ ID NO:1939 is the determined cDNA sequence for clone 62177475 R0393:E06

SEQ ID NO:1940 is the determined cDNA sequence for clone 62177476 R0393:E07

SEQ ID NO:1941 is the determined cDNA sequence for clone 62177477 R0393:E08

SEQ ID NO:1942 is the determined cDNA sequence for clone 62177478 R0393:E09

SEQ ID NO:1943 is the determined cDNA sequence for clone 62177480 R0393:E11

SEQ ID NO:1944 is the determined cDNA sequence for clone 62177481 R0393:E12

SEQ ID NO:1945 is the determined cDNA sequence for clone 62177482 R0393:F01

SEQ ID NO:1946 is the determined cDNA sequence for clone 62177483 R0393:F02

SEQ ID NO:1947 is the determined cDNA sequence for clone 62177484 R0393:F03

SEQ ID NO:1948 is the determined cDNA sequence for clone 62177485 R0393:F04

SEQ ID NO:1949 is the determined cDNA sequence for clone 62177486 R0393:F05

SEQ ID NO:1950 is the determined cDNA sequence for clone 62177487 R0393:F06

SEQ ID NO:1951 is the determined cDNA sequence for clone 62177488 R0393:F07

SEQ ID NO:1952 is the determined cDNA sequence for clone 62177489 R0393:F08

SEQ ID NO:1953 is the determined cDNA sequence for clone 62177490 R0393:F09

SEQ ID NO:1954 is the determined cDNA sequence for clone 62177491 R0393:F10

SEQ ID NO:1955 is the determined cDNA sequence for clone 62177492 R0393:F11

SEQ ID NO:1956 is the determined cDNA sequence for clone 62177493 R0393:F12

SEQ ID NO:1957 is the determined cDNA sequence for clone 62177494 R0393:G01

SEQ ID NO:1958 is the determined cDNA sequence for clone 62177495 R0393:G02

SEQ ID NO:1959 is the determined cDNA sequence for clone 62177496 R0393:G03

SEQ ID NO:1960 is the determined cDNA sequence for clone 62177497 R0393:G04

SEQ ID NO:1961 is the determined cDNA sequence for clone 62177498 R0393:G05

SEQ ID NO:1962 is the determined cDNA sequence for clone 62177499 R0393:G06

SEQ ID NO:1963 is the determined cDNA sequence for clone 62177500 R0393:G07

SEQ ID NO:1964 is the determined cDNA sequence for clone 62177501 R0393:G08

SEQ ID NO:1965 is the determined cDNA sequence for clone 62177502 R0393:G09

SEQ ID NO:1966 is the determined cDNA sequence for clone 62177503 R0393:G10

SEQ ID NO:1967 is the determined cDNA sequence for clone 62177504 R0393:G11

SEQ ID NO:1968 is the determined cDNA sequence for clone 62177505 R0393:G12

SEQ ID NO:1969 is the determined cDNA sequence for clone 62177506 R0393:H01

SEQ ID NO:1970 is the determined cDNA sequence for clone 62177507 R0393:H02

SEQ ID NO:1971 is the determined cDNA sequence for clone 62177509 R0393:H04

SEQ ID NO:1972 is the determined cDNA sequence for clone 62177510 R0393:H05

SEQ ID NO:1973 is the determined cDNA sequence for clone 62177511 R0393:H06

SEQ ID NO:1974 is the determined cDNA sequence for clone 62177513 R0393:H08

SEQ ID NO:1975 is the determined cDNA sequence for clone 62177514 R0393:H09

SEQ ID NO:1976 is the determined cDNA sequence for clone 62177515 R0393:H10

SEQ ID NO:1977 is the determined cDNA sequence for clone 62177516 R0393:H11

SEQ ID NO:1978 is the determined cDNA sequence for clone 62227153 R0394:A02

SEQ ID NO:1979 is the determined cDNA sequence for clone 62227154 R0394:A03

SEQ ID NO:1980 is the determined cDNA sequence for clone 62227155 R0394:A05

SEQ ID NO:1981 is the determined cDNA sequence for clone 62227156 R0394:A06

SEQ ID NO:1982 is the determined cDNA sequence for clone 62227157 R0394:A07

SEQ ID NO:1983 is the determined cDNA sequence for clone 62227158 R0394:A08

SEQ ID NO:1984 is the determined cDNA sequence for clone 62227159 R0394:A09

SEQ ID NO:1985 is the determined cDNA sequence for clone 62227160 R0394:A10

SEQ ID NO:1986 is the determined cDNA sequence for clone 62227161 R0394:A11

SEQ ID NO:1987 is the determined cDNA sequence for clone 62227162 R0394:A12

SEQ ID NO:1988 is the determined cDNA sequence for clone 62227164 R0394:B02

SEQ ID NO:1989 is the determined cDNA sequence for clone 62227165 R0394:B03

SEQ ID NO:1990 is the determined cDNA sequence for clone 62227166 R0394:B04

SEQ ID NO:1991 is the determined cDNA sequence for clone 62227167 R0394:B05

SEQ ID NO:1992 is the determined cDNA sequence for clone 62227169 R0394:B07

SEQ ID NO:1993 is the determined cDNA sequence for clone 62227170 R0394:B08

SEQ ID NO:1994 is the determined cDNA sequence for clone 62227171 R0394:B09

SEQ ID NO:1995 is the determined cDNA sequence for clone 62227172 R0394:B10

SEQ ID NO:1996 is the determined cDNA sequence for clone 62227173 R0394:B11

SEQ ID NO:1997 is the determined cDNA sequence for clone 62227174 R0394:B12

SEQ ID NO:1998 is the determined cDNA sequence for clone 62227175 R0394:C01

SEQ ID NO:1999 is the determined cDNA sequence for clone 62227177 R0394:C03

SEQ ID NO:2000 is the determined cDNA sequence for clone 62227178 R0394:C04

SEQ ID NO:2001 is the determined cDNA sequence for clone 62227179 R0394:C05

SEQ ID NO:2002 is the determined cDNA sequence for clone 62227180 R0394:C06

SEQ ID NO:2003 is the determined cDNA sequence for clone 62227181 R0394:C07

SEQ ID NO:2004 is the determined cDNA sequence for clone 62227182 R0394:C08

SEQ ID NO:2005 is the determined cDNA sequence for clone 62227183 R0394:C09

SEQ ID NO:2006 is the determined cDNA sequence for clone 62227185 R0394:C11

SEQ ID NO:2007 is the determined cDNA sequence for clone 62227187 R0394:D01

SEQ ID NO:2008 is the determined cDNA sequence for clone 62227188 R0394:D02

SEQ ID NO:2009 is the determined cDNA sequence for clone 62227189 R0394:D03

SEQ ID NO:2010 is the determined cDNA sequence for clone 62227190 R0394:D04

SEQ ID NO:2011 is the determined cDNA sequence for clone 62227191 R0394:D05

SEQ ID NO:2012 is the determined cDNA sequence for clone 62227193 R0394:D07

SEQ ID NO:2013 is the determined cDNA sequence for clone 62227194 R0394:D08

SEQ ID NO:2014 is the determined cDNA sequence for clone 62227195 R0394:D09

SEQ ID NO:2015 is the determined cDNA sequence for clone 62227197 R0394:D11

SEQ ID NO:2016 is the determined cDNA sequence for clone 62227198 R0394:D12

SEQ ID NO:2017 is the determined cDNA sequence for clone 62227199 R0394:E01

SEQ ID NO:2018 is the determined cDNA sequence for clone 62227200 R0394:E02

SEQ ID NO:2019 is the determined cDNA sequence for clone 62227201 R0394:E03

SEQ ID NO:2020 is the determined cDNA sequence for clone 62227202 R0394:E04

SEQ ID NO:2021 is the determined cDNA sequence for clone 62227203 R0394:E05

SEQ ID NO:2022 is the determined cDNA sequence for clone 62227204 R0394:E06

SEQ ID NO:2023 is the determined cDNA sequence for clone 62227205 R0394:E07

SEQ ID NO:2024 is the determined cDNA sequence for clone 62227206 R0394:E08

SEQ ID NO:2025 is the determined cDNA sequence for clone 62227207 R0394:E09

SEQ ID NO:2026 is the determined cDNA sequence for clone 62227208 R0394:E10

SEQ ID NO:2027 is the determined cDNA sequence for clone 62227209 R0394:E11

SEQ ID NO:2028 is the determined cDNA sequence for clone 62227210 R0394:E12

SEQ ID NO:2029 is the determined cDNA sequence for clone 62227212 R0394:F02

SEQ ID NO:2030 is the determined cDNA sequence for clone 62227213 R0394:F03

SEQ ID NO:2031 is the determined cDNA sequence for clone 62227214 R0394:F04

SEQ ID NO:2032 is the determined cDNA sequence for clone 62227216 R0394:F06

SEQ ID NO:2033 is the determined cDNA sequence for clone 62227217 R0394:F07

SEQ ID NO:2034 is the determined cDNA sequence for clone 62227218 R0394:F08

SEQ ID NO:2035 is the determined cDNA sequence for clone 62227219 R0394:F09

SEQ ID NO:2036 is the determined cDNA sequence for clone 62227220 R0394:F10

SEQ ID NO:2037 is the determined cDNA sequence for clone 62227221 R0394:F11

SEQ ID NO:2038 is the determined cDNA sequence for clone 62227222 R0394:F02

SEQ ID NO:2039 is the determined cDNA sequence for clone 62227223 R0394:G01

SEQ ID NO:2040 is the determined cDNA sequence for clone 62227224 R0394:G02

SEQ ID NO:2041 is the determined cDNA sequence for clone 62227226 R0394:G04

SEQ ID NO:2042 is the determined cDNA sequence for clone 62227229 R0394:G07

SEQ ID NO:2043 is the determined cDNA sequence for clone 62227230 R0394:G08

SEQ ID NO:2044 is the determined cDNA sequence for clone 62227231 R0394:G09

SEQ ID NO:2045 is the determined cDNA sequence for clone 62227232 R0394:G10

SEQ ID NO:2046 is the determined cDNA sequence for clone 62227233 R0394:G11

SEQ ID NO:2047 is the determined cDNA sequence for clone 62227234 R0394:G12

SEQ ID NO:2048 is the determined cDNA sequence for clone 62227236 R0394:H02

SEQ ID NO:2049 is the determined cDNA sequence for clone 62227237 R0394:H03

SEQ ID NO:2050 is the determined cDNA sequence for clone 62227238 R0394:H04

SEQ ID NO:2051 is the determined cDNA sequence for clone 62227239 R0394:H05

SEQ ID NO:2052 is the determined cDNA sequence for clone 62227241 R0394:H07

SEQ ID NO:2053 is the determined cDNA sequence for clone 62227242 R0394:H08

SEQ ID NO:2054 is the determined cDNA sequence for clone 62227243 R0394:H09

SEQ ID NO:2055 is the determined cDNA sequence for clone 62227244 R0394:H10

SEQ ID NO:2056 is the determined cDNA sequence for clone 62227245 R0394:H11

SEQ ID NO:2057 is the determined cDNA sequence for clone 62296724 R0395:A03

SEQ ID NO:2058 is the determined cDNA sequence for clone 62296725 R0395:A05

SEQ ID NO:2059 is the determined cDNA sequence for clone 62296726 R0395:A06

SEQ ID NO:2060 is the determined cDNA sequence for clone 62296727 R0395:A07

SEQ ID NO:2061 is the determined cDNA sequence for clone 62296729 R0395:A09

SEQ ID NO:2062 is the determined cDNA sequence for clone 62296730 R0395:A10

SEQ ID NO:2063 is the determined cDNA sequence for clone 62296731 R0395:A11

SEQ ID NO:2064 is the determined cDNA sequence for clone 62296733 R0395:B01

SEQ ID NO:2065 is the determined cDNA sequence for clone 62296734 R0395:B02

SEQ ID NO:2066 is the determined cDNA sequence for clone 62296735 R0395:B03

SEQ ID NO:2067 is the determined cDNA sequence for clone 62296736 R0395:B04

SEQ ID NO:2068 is the determined cDNA sequence for clone 62296737 R0395:B05

SEQ ID NO:2069 is the determined cDNA sequence for clone 62296738 R0395:B06

SEQ ID NO:2070 is the determined cDNA sequence for clone 62296739 R0395:B07

SEQ ID NO:2071 is the determined cDNA sequence for clone 62296740 R0395:B08

SEQ ID NO:2072 is the determined cDNA sequence for clone 62296741 R0395:B09

SEQ ID NO:2073 is the determined cDNA sequence for clone 62296742 R0395:B10

SEQ ID NO:2074 is the determined cDNA sequence for clone 62296743 R0395:B11

SEQ ID NO:2075 is the determined cDNA sequence for clone 62296744 R0395:B12

SEQ ID NO:2076 is the determined cDNA sequence for clone 62296746 R0395:C02

SEQ ID NO:2077 is the determined cDNA sequence for clone 62296747 R0395:C03

SEQ ID NO:2078 is the determined cDNA sequence for clone 62296748 R0395:C04

SEQ ID NO:2079 is the determined cDNA sequence for clone 62296750 R0395:C06

SEQ ID NO:2080 is the determined cDNA sequence for clone 62296751 R0395:C07

SEQ ID NO:2081 is the determined cDNA sequence for clone 62296752 R0395:C08

SEQ ID NO:2082 is the determined cDNA sequence for clone 62296753 R0395:C09

SEQ ID NO:2083 is the determined cDNA sequence for clone 62296754 R0395:C10

SEQ ID NO:2084 is the determined cDNA sequence for clone 62296755 R0395:C11

SEQ ID NO:2085 is the determined cDNA sequence for clone 62296756 R0395:C12

SEQ ID NO:2086 is the determined cDNA sequence for clone 62296757 R0395:D01

SEQ ID NO:2087 is the determined cDNA sequence for clone 62296758 R0395:D02

SEQ ID NO:2088 is the determined cDNA sequence for clone 62296759 R0395:D03

SEQ ID NO:2089 is the determined cDNA sequence for clone 62296760 R0395:D04

SEQ ID NO:2090 is the determined cDNA sequence for clone 62296761 R0395:D05

SEQ ID NO:2091 is the determined cDNA sequence for clone 62296762 R0395:D06

SEQ ID NO:2092 is the determined cDNA sequence for clone 62296763 R0395:D07

SEQ ID NO:2093 is the determined cDNA sequence for clone 62296764 R0395:D08

SEQ ID NO:2094 is the determined cDNA sequence for clone 62296765 R0395:D09

SEQ ID NO:2095 is the determined cDNA sequence for clone 62296766 R0395:D10

SEQ ID NO:2096 is the determined cDNA sequence for clone 62296767 R0395:D11

SEQ ID NO:2097 is the determined cDNA sequence for clone 62296768 R0395:D12

SEQ ID NO:2098 is the determined cDNA sequence for clone 62296769 R0395:E01

SEQ ID NO:2099 is the determined cDNA sequence for clone 62296770 R0395:E02

SEQ ID NO:2100 is the determined cDNA sequence for clone 62296771 R0395:E03

SEQ ID NO:2101 is the determined cDNA sequence for clone 62296772 R0395:E04

SEQ ID NO:2102 is the determined cDNA sequence for clone 62296773 R0395:E05

SEQ ID NO:2103 is the determined cDNA sequence for clone 62296774 R0395:E06

SEQ ID NO:2104 is the determined cDNA sequence for clone 62296775 R0395:E07

SEQ ID NO:2105 is the determined cDNA sequence for clone 62296776 R0395:E08

SEQ ID NO:2106 is the determined cDNA sequence for clone 62296777 R0395:E09

SEQ ID NO:2107 is the determined cDNA sequence for clone 62296778 R0395:E10

SEQ ID NO:2108 is the determined cDNA sequence for clone 62296779 R0395:E11

SEQ ID NO:2109 is the determined cDNA sequence for clone 62296780 R0395:E12

SEQ ID NO:2110 is the determined cDNA sequence for clone 62296781 R0395:F01

SEQ ID NO:2111 is the determined cDNA sequence for clone 62296783 R0395:F03

SEQ ID NO:2112 is the determined cDNA sequence for clone 62296785 R0395:F05

SEQ ID NO:2113 is the determined cDNA sequence for clone 62296786 R0395:F06

SEQ ID NO:2114 is the determined cDNA sequence for clone 62296787 R0395:F07

SEQ ID NO:2115 is the determined cDNA sequence for clone 62296788 R0395:F08

SEQ ID NO:2116 is the determined cDNA sequence for clone 62296789 R0395:F09

SEQ ID NO:2117 is the determined cDNA sequence for clone 62296790 R0395:F10

SEQ ID NO:2118 is the determined cDNA sequence for clone 62296791 R0395:F11

SEQ ID NO:2119 is the determined cDNA sequence for clone 62296792 R0395:F12

SEQ ID NO:2120 is the determined cDNA sequence for clone 62296795 R0395:G03

SEQ ID NO:2121 is the determined cDNA sequence for clone 62296796 R0395:G04

SEQ ID NO:2122 is the determined cDNA sequence for clone 62296798 R0395:G06

SEQ ID NO:2123 is the determined cDNA sequence for clone 62296799 R0395:G07

SEQ ID NO:2124 is the determined cDNA sequence for clone 62296801 R0395:G09

SEQ ID NO:2125 is the determined cDNA sequence for clone 62296802 R0395:G10

SEQ ID NO:2126 is the determined cDNA sequence for clone 62296803 R0395:G11

SEQ ID NO:2127 is the determined cDNA sequence for clone 62296804 R0395:G12

SEQ ID NO:2128 is the determined cDNA sequence for clone 62296805 R0395:H01

SEQ ID NO:2129 is the determined cDNA sequence for clone 62296807 R0395:H03

SEQ ID NO:2130 is the determined cDNA sequence for clone 62296808 R0395:H04

SEQ ID NO:2131 is the determined cDNA sequence for clone 62296810 R0395:H06

SEQ ID NO:2132 is the determined cDNA sequence for clone 62296811 R0395:H07

SEQ ID NO:2133 is the determined cDNA sequence for clone 62296812 R0395:H08

SEQ ID NO:2134 is the determined cDNA sequence for clone 62296814 R0395:H10

SEQ ID NO:2135 is the determined cDNA sequence for clone 62296815 R0395:H11

SEQ ID NO:2136 is the determined cDNA sequence for clone 62227061 R0396:A03

SEQ ID NO:2137 is the determined cDNA sequence for clone 62227062 R0396:A05

SEQ ID NO:2138 is the determined cDNA sequence for clone 62227063 R0396:A06

SEQ ID NO:2139 is the determined cDNA sequence for clone 62227064 R0396:A07

SEQ ID NO:2140 is the determined cDNA sequence for clone 62227065 R0396:A08

SEQ ID NO:2141 is the determined cDNA sequence for clone 62227066 R0396:A09

SEQ ID NO:2142 is the determined cDNA sequence for clone 62227067 R0396:A10

SEQ ID NO:2143 is the determined cDNA sequence for clone 62227068 R0396:A11

SEQ ID NO:2144 is the determined cDNA sequence for clone 62227069 R0396:A12

SEQ ID NO:2145 is the determined cDNA sequence for clone 62227070 R0396:B01

SEQ ID NO:2146 is the determined cDNA sequence for clone 62227071 R0396:B02

SEQ ID NO:2147 is the determined cDNA sequence for clone 62227072 R0396:B03

SEQ ID NO:2148 is the determined cDNA sequence for clone 62227074 R0396:B05

SEQ ID NO:2149 is the determined cDNA sequence for clone 62227076 R0396:B07

SEQ ID NO:2150 is the determined cDNA sequence for clone 62227078 R0396:B09

SEQ ID NO:2151 is the determined cDNA sequence for clone 62227079 R0396:B10

SEQ ID NO:2152 is the determined cDNA sequence for clone 62227080 R0396:B11

SEQ ID NO:2153 is the determined cDNA sequence for clone 62227081 R0396:B12

SEQ ID NO:2154 is the determined cDNA sequence for clone 62227082 R0396:C01

SEQ ID NO:2155 is the determined cDNA sequence for clone 62227083 R0396:C02

SEQ ID NO:2156 is the determined cDNA sequence for clone 62227085 R0396:C04

SEQ ID NO:2157 is the determined cDNA sequence for clone 62227086 R0396:C05

SEQ ID NO:2158 is the determined cDNA sequence for clone 62227087 R0396:C06

SEQ ID NO:2159 is the determined cDNA sequence for clone 62227088 R0396:C07

SEQ ID NO:2160 is the determined cDNA sequence for clone 62227091 R0396:C10

SEQ ID NO:2161 is the determined cDNA sequence for clone 62227092 R0396:C11

SEQ ID NO:2162 is the determined cDNA sequence for clone 62227093 R0396:C12

SEQ ID NO:2163 is the determined cDNA sequence for clone 62227094 R0396:D01

SEQ ID NO:2164 is the determined cDNA sequence for clone 62227095 R0396:D02

SEQ ID NO:2165 is the determined cDNA sequence for clone 62227096 R0396:D03

SEQ ID NO:2166 is the determined cDNA sequence for clone 62227097 R0396:D04

SEQ ID NO:2167 is the determined cDNA sequence for clone 62227098 R0396:D05

SEQ ID NO:2168 is the determined cDNA sequence for clone 62227099 R0396:D06

SEQ ID NO:2169 is the determined cDNA sequence for clone 62227100 R0396:D07

SEQ ID NO:2170 is the determined cDNA sequence for clone 62227101 R0396:D08

SEQ ID NO:2171 is the determined cDNA sequence for clone 62227102 R0396:D09

SEQ ID NO:2172 is the determined cDNA sequence for clone 62227103 R0396:D10

SEQ ID NO:2173 is the determined cDNA sequence for clone 62227104 R0396:D11

SEQ ID NO:2174 is the determined cDNA sequence for clone 62227105 R0396:D12

SEQ ID NO:2175 is the determined cDNA sequence for clone 62227107 R0396:E02

SEQ ID NO:2176 is the determined cDNA sequence for clone 62227109 R0396:E04

SEQ ID NO:2177 is the determined cDNA sequence for clone 62227110 R0396:E05

SEQ ID NO:2178 is the determined cDNA sequence for clone 62227111 R0396:E06

SEQ ID NO:2179 is the determined cDNA sequence for clone 62227112 R0396:E07

SEQ ID NO:2180 is the determined cDNA sequence for clone 62227114 R0396:E09

SEQ ID NO:2181 is the determined cDNA sequence for clone 62227115 R0396:E10

SEQ ID NO:2182 is the determined cDNA sequence for clone 62227116 R0396:E11

SEQ ID NO:2183 is the determined cDNA sequence for clone 62227117 R0396:E12

SEQ ID NO:2184 is the determined cDNA sequence for clone 62227118 R0396:F01

SEQ ID NO:2185 is the determined cDNA sequence for clone 62227119 R0396:F02

SEQ ID NO:2186 is the determined cDNA sequence for clone 62227120 R0396:F03

SEQ ID NO:2187 is the determined cDNA sequence for clone 62227121 R0396:F04

SEQ ID NO:2188 is the determined cDNA sequence for clone 62227123 R0396:F06

SEQ ID NO:2189 is the determined cDNA sequence for clone 62227124 R0396:F07

SEQ ID NO:2190 is the determined cDNA sequence for clone 62227125 R0396:F08

SEQ ID NO:2191 is the determined cDNA sequence for clone 62227126 R0396:F09

SEQ ID NO:2192 is the determined cDNA sequence for clone 62227127 R0396:F10

SEQ ID NO:2193 is the determined cDNA sequence for clone 62227128 R0396:F11

SEQ ID NO:2194 is the determined cDNA sequence for clone 62227129 R0396:F12

SEQ ID NO:2195 is the determined cDNA sequence for clone 62227130 R0396:G01

SEQ ID NO:2196 is the determined cDNA sequence for clone 62227133 R0396:G04

SEQ ID NO:2197 is the determined cDNA sequence for clone 62227134 R0396:G05

SEQ ID NO:2198 is the determined cDNA sequence for clone 62227135 R0396:G06

SEQ ID NO:2199 is the determined cDNA sequence for clone 62227136 R0396:G07

SEQ ID NO:2200 is the determined cDNA sequence for clone 62227138 R0396:G09

SEQ ID NO:2201 is the determined cDNA sequence for clone 62227139 R0396:G10

SEQ ID NO:2202 is the determined cDNA sequence for clone 62227140 R0396:G11

SEQ ID NO:2203 is the determined cDNA sequence for clone 62227141 R0396:G12

SEQ ID NO:2204 is the determined cDNA sequence for clone 62227142 R0396:H01

SEQ ID NO:2205 is the determined cDNA sequence for clone 62227144 R0396:H03

SEQ ID NO:2206 is the determined cDNA sequence for clone 62227145 R0396:H04

SEQ ID NO:2207 is the determined cDNA sequence for clone 62227147 R0396:H06

SEQ ID NO:2208 is the determined cDNA sequence for clone 62227148 R0396:H07

SEQ ID NO:2209 is the determined cDNA sequence for clone 62227149 R0396:H08

SEQ ID NO:2210 is the determined cDNA sequence for clone 62227151 R0396:H10

SEQ ID NO:2211 is the determined cDNA sequence for clone 62227152 R0396:H11

SEQ ID NO:2212 is the determined cDNA sequence for clone 62296817 R0397:A03

SEQ ID NO:2213 is the determined cDNA sequence for clone 62296818 R0397:A05

SEQ ID NO:2214 is the determined cDNA sequence for clone 62296820 R0397:A07

SEQ ID NO:2215 is the determined cDNA sequence for clone 62296821 R0397:A08

SEQ ID NO:2216 is the determined cDNA sequence for clone 62296822 R0397:A09

SEQ ID NO:2217 is the determined cDNA sequence for clone 62296824 R0397:A11

SEQ ID NO:2218 is the determined cDNA sequence for clone 62296825 R0397:A12

SEQ ID NO:2219 is the determined cDNA sequence for clone 62296827 R0397:B02

SEQ ID NO:2220 is the determined cDNA sequence for clone 62296828 R0397:B03

SEQ ID NO:2221 is the determined cDNA sequence for clone 62296829 R0397:B04

SEQ ID NO:2222 is the determined cDNA sequence for clone 62296830 R0397:B05

SEQ ID NO:2223 is the determined cDNA sequence for clone 62296831 R0397:B06

SEQ ID NO:2224 is the determined cDNA sequence for clone 62296832 R0397:B07

SEQ ID NO:2225 is the determined cDNA sequence for clone 62296834 R0397:B09

SEQ ID NO:2226 is the determined cDNA sequence for clone 62296835 R0397:B10

SEQ ID NO:2227 is the determined cDNA sequence for clone 62296836 R0397:B11

SEQ ID NO:2228 is the determined cDNA sequence for clone 62296837 R0397:B12

SEQ ID NO:2229 is the determined cDNA sequence for clone 62296838 R0397:C01

SEQ ID NO:2230 is the determined cDNA sequence for clone 62296840 R0397:C03

SEQ ID NO:2231 is the determined cDNA sequence for clone 62296841 R0397:C04

SEQ ID NO:2232 is the determined cDNA sequence for clone 62296842 R0397:C05

SEQ ID NO:2233 is the determined cDNA sequence for clone 62296843 R0397:C06

SEQ ID NO:2234 is the determined cDNA sequence for clone 62296844 R0397:C07

SEQ ID NO:2235 is the determined cDNA sequence for clone 62296845 R0397:C08

SEQ ID NO:2236 is the determined cDNA sequence for clone 62296846 R0397:C09

SEQ ID NO:2237 is the determined cDNA sequence for clone 62296847 R0397:C10

SEQ ID NO:2238 is the determined cDNA sequence for clone 62296848 R0397:C11

SEQ ID NO:2239 is the determined cDNA sequence for clone 62296849 R0397:C12

SEQ ID NO:2240 is the determined cDNA sequence for clone 62296851 R0397:D02

SEQ ID NO:2241 is the determined cDNA sequence for clone 62296854 R0397:D05

SEQ ID NO:2242 is the determined cDNA sequence for clone 62296855 R0397:D06

SEQ ID NO:2243 is the determined cDNA sequence for clone 62296856 R0397:D07

SEQ ID NO:2244 is the determined cDNA sequence for clone 62296857 R0397:D08

SEQ ID NO:2245 is the determined cDNA sequence for clone 62296858 R0397:D09

SEQ ID NO:2246 is the determined cDNA sequence for clone 62296860 R0397:D11

SEQ ID NO:2247 is the determined cDNA sequence for clone 62296861 R0397:D12

SEQ ID NO:2248 is the determined cDNA sequence for clone 62296862 R0397:E01

SEQ ID NO:2249 is the determined cDNA sequence for clone 62296864 R0397:E03

SEQ ID NO:2250 is the determined cDNA sequence for clone 62296865 R0397:E04

SEQ ID NO:2251 is the determined cDNA sequence for clone 62296866 R0397:E05

SEQ ID NO:2252 is the determined cDNA sequence for clone 62296867 R0397:E06

SEQ ID NO:2253 is the determined cDNA sequence for clone 62296869 R0397:E08

SEQ ID NO:2254 is the determined cDNA sequence for clone 62296870 R0397:E09

SEQ ID NO:2255 is the determined cDNA sequence for clone 62296871 R0397:E10

SEQ ID NO:2256 is the determined cDNA sequence for clone 62296872 R0397:E11

SEQ ID NO:2257 is the determined cDNA sequence for clone 62296873 R0397:E12

SEQ ID NO:2258 is the determined cDNA sequence for clone 62296874 R0397:F01

SEQ ID NO:2259 is the determined cDNA sequence for clone 62296875 R0397:F02

SEQ ID NO:2260 is the determined cDNA sequence for clone 62296876 R0397:F03

SEQ ID NO:2261 is the determined cDNA sequence for clone 62296877 R0397:F04

SEQ ID NO:2262 is the determined cDNA sequence for clone 62296878 R0397:F05

SEQ ID NO:2263 is the determined cDNA sequence for clone 62296879 R0397:F06

SEQ ID NO:2264 is the determined cDNA sequence for clone 62296880 R0397:F07

SEQ ID NO:2265 is the determined cDNA sequence for clone 62296881 R0397:F08

SEQ ID NO:2266 is the determined cDNA sequence for clone 62296882 R0397:F09

SEQ ID NO:2267 is the determined cDNA sequence for clone 62296883 R0397:F10

SEQ ID NO:2268 is the determined cDNA sequence for clone 62296884 R0397:F11

SEQ ID NO:2269 is the determined cDNA sequence for clone 62296885 R0397:F12

SEQ ID NO:2270 is the determined cDNA sequence for clone 62296886 R0397:G01

SEQ ID NO:2271 is the determined cDNA sequence for clone 62296887 R0397:G02

SEQ ID NO:2272 is the determined cDNA sequence for clone 62296889 R0397:G04

SEQ ID NO:2273 is the determined cDNA sequence for clone 62296890 R0397:G05

SEQ ID NO:2274 is the determined cDNA sequence for clone 62296891 R0397:G06

SEQ ID NO:2275 is the determined cDNA sequence for clone 62296892 R0397:G07

SEQ ID NO:2276 is the determined cDNA sequence for clone 62296893 R0397:G08

SEQ ID NO:2277 is the determined cDNA sequence for clone 62296895 R0397:G10

SEQ ID NO:2278 is the determined cDNA sequence for clone 62296897 R0397:G12

SEQ ID NO:2279 is the determined cDNA sequence for clone 62296898 R0397:H01

SEQ ID NO:2280 is the determined cDNA sequence for clone 62296899 R0397:H02

SEQ ID NO:2281 is the determined cDNA sequence for clone 62296900 R0397:H03

SEQ ID NO:2282 is the determined cDNA sequence for clone 62296902 R0397:H05

SEQ ID NO:2283 is the determined cDNA sequence for clone 62296904 R0397:H07

SEQ ID NO:2284 is the determined cDNA sequence for clone 62296905 R0397:H08

SEQ ID NO:2285 is the determined cDNA sequence for clone 62296906 R0397:H09

SEQ ID NO:2286 is the determined cDNA sequence for clone 62296907 R0397:H10

SEQ ID NO:2287 is the determined cDNA sequence for clone 62296908 R0397:H11

SEQ ID NO:2288 is the determined cDNA sequence for clone 62328788 R0398:A02

SEQ ID NO:2289 is the determined cDNA sequence for clone 62328789 R0398:A03

SEQ ID NO:2290 is the determined cDNA sequence for clone 62328790 R0398:A05

SEQ ID NO:2291 is the determined cDNA sequence for clone 62328791 R0398:A06

SEQ ID NO:2292 is the determined cDNA sequence for clone 62328792 R0398:A07

SEQ ID NO:2293 is the determined cDNA sequence for clone 62328794 R0398:A09

SEQ ID NO:2294 is the determined cDNA sequence for clone 62328795 R0398:A10

SEQ ID NO:2295 is the determined cDNA sequence for clone 62328796 R0398:A11

SEQ ID NO:2296 is the determined cDNA sequence for clone 62328798 R0398:B01

SEQ ID NO:2297 is the determined cDNA sequence for clone 62328799 R0398:B02

SEQ ID NO:2298 is the determined cDNA sequence for clone 62328800 R0398:B03

SEQ ID NO:2299 is the determined cDNA sequence for clone 62328802 R0398:B05

SEQ ID NO:2300 is the determined cDNA sequence for clone 62328803 R0398:B06

SEQ ID NO:2301 is the determined cDNA sequence for clone 62328804 R0398:B07

SEQ ID NO:2302 is the determined cDNA sequence for clone 62328805 R0398:B08

SEQ ID NO:2303 is the determined cDNA sequence for clone 62328806 R0398:B09

SEQ ID NO:2304 is the determined cDNA sequence for clone 62328807 R0398:B10

SEQ ID NO:2305 is the determined cDNA sequence for clone 62328808 R0398:B11

SEQ ID NO:2306 is the determined cDNA sequence for clone 62328809 R0398:B12

SEQ ID NO:2307 is the determined cDNA sequence for clone 62328810 R0398:C01

SEQ ID NO:2308 is the determined cDNA sequence for clone 62328811 R0398:C02

SEQ ID NO:2309 is the determined cDNA sequence for clone 62328814 R0398:C05

SEQ ID NO:2310 is the determined cDNA sequence for clone 62328815 R0398:C06

SEQ ID NO:2311 is the determined cDNA sequence for clone 62328816 R0398:C07

SEQ ID NO:2312 is the determined cDNA sequence for clone 62328817 R0398:C08

SEQ ID NO:2313 is the determined cDNA sequence for clone 62328818 R0398:C09

SEQ ID NO:2314 is the determined cDNA sequence for clone 62328819 R0398:C10

SEQ ID NO:2315 is the determined cDNA sequence for clone 62328820 R0398:C11

SEQ ID NO:2316 is the determined cDNA sequence for clone 62328821 R0398:C12

SEQ ID NO:2317 is the determined cDNA sequence for clone 62328822 R0398:D01

SEQ ID NO:2318 is the determined cDNA sequence for clone 62328823 R0398:D02

SEQ ID NO:2319 is the determined cDNA sequence for clone 62328826 R0398:D05

SEQ ID NO:2320 is the determined cDNA sequence for clone 62328827 R0398:D06

SEQ ID NO:2321 is the determined cDNA sequence for clone 62328828 R0398:D07

SEQ ID NO:2322 is the determined cDNA sequence for clone 62328829 R0398:D08

SEQ ID NO:2323 is the determined cDNA sequence for clone 62328830 R0398:D09

SEQ ID NO:2324 is the determined cDNA sequence for clone 62328831 R0398:D10

SEQ ID NO:2325 is the determined cDNA sequence for clone 62328832 R0398:D11

SEQ ID NO:2326 is the determined cDNA sequence for clone 62328833 R0398:D12

SEQ ID NO:2327 is the determined cDNA sequence for clone 62328834 R0398:E01

SEQ ID NO:2328 is the determined cDNA sequence for clone 62328836 R0398:E03

SEQ ID NO:2329 is the determined cDNA sequence for clone 62328837 R0398:E04

SEQ ID NO:2330 is the determined cDNA sequence for clone 62328838 R0398:E05

SEQ ID NO:2331 is the determined cDNA sequence for clone 62328839 R0398:E06

SEQ ID NO:2332 is the determined cDNA sequence for clone 62328840 R0398:E07

SEQ ID NO:2333 is the determined cDNA sequence for clone 62328841 R0398:E08

SEQ ID NO:2334 is the determined cDNA sequence for clone 62328842 R0398:E09

SEQ ID NO:2335 is the determined cDNA sequence for clone 62328843 R0398:E10

SEQ ID NO:2336 is the determined cDNA sequence for clone 62328844 R0398:E11

SEQ ID NO:2337 is the determined cDNA sequence for clone 62328845 R0398:E12

SEQ ID NO:2338 is the determined cDNA sequence for clone 62328846 R0398:F01

SEQ ID NO:2339 is the determined cDNA sequence for clone 62328847 R0398:F02

SEQ ID NO:2340 is the determined cDNA sequence for clone 62328848 R0398:F03

SEQ ID NO:2341 is the determined cDNA sequence for clone 62328849 R0398:F04

SEQ ID NO:2342 is the determined cDNA sequence for clone 62328850 R0398:F05

SEQ ID NO:2343 is the determined cDNA sequence for clone 62328851 R0398:F06

SEQ ID NO:2344 is the determined cDNA sequence for clone 62328852 R0398:F07

SEQ ID NO:2345 is the determined cDNA sequence for clone 62328853 R0398:F08

SEQ ID NO:2346 is the determined cDNA sequence for clone 62328855 R0398:F10

SEQ ID NO:2347 is the determined cDNA sequence for clone 62328856 R0398:F11

SEQ ID NO:2348 is the determined cDNA sequence for clone 62328857 R0398:F12

SEQ ID NO:2349 is the determined cDNA sequence for clone 62328858 R0398:G01

SEQ ID NO:2350 is the determined cDNA sequence for clone 62328859 R0398:G02

SEQ ID NO:2351 is the determined cDNA sequence for clone 62328860 R0398:G03

SEQ ID NO:2352 is the determined cDNA sequence for clone 62328861 R0398:G04

SEQ ID NO:2353 is the determined cDNA sequence for clone 62328862 R0398:G05

SEQ ID NO:2354 is the determined cDNA sequence for clone 62328863 R0398:G06

SEQ ID NO:2355 is the determined cDNA sequence for clone 62328865 R0398:G08

SEQ ID NO:2356 is the determined cDNA sequence for clone 62328866 R0398:G09

SEQ ID NO:2357 is the determined cDNA sequence for clone 62328867 R0398:G10

SEQ ID NO:2358 is the determined cDNA sequence for clone 62328868 R0398:G11

SEQ ID NO:2359 is the determined cDNA sequence for clone 62328869 R0398:G12

SEQ ID NO:2360 is the determined cDNA sequence for clone 62328870 R0398:H01

SEQ ID NO:2361 is the determined cDNA sequence for clone 62328871 R0398:H02

SEQ ID NO:2362 is the determined cDNA sequence for clone 62328872 R0398:H03

SEQ ID NO:2363 is the determined cDNA sequence for clone 62328873 R0398:H04

SEQ ID NO:2364 is the determined cDNA sequence for clone 62328874 R0398:H05

SEQ ID NO:2365 is the determined cDNA sequence for clone 62328875 R0398:H06

SEQ ID NO:2366 is the determined cDNA sequence for clone 62328876 R0398:H07

SEQ ID NO:2367 is the determined cDNA sequence for clone 62328877 R0398:H08

SEQ ID NO:2368 is the determined cDNA sequence for clone 62328878 R0398:H09

SEQ ID NO:2369 is the determined cDNA sequence for clone 62328879 R0398:H10

SEQ ID NO:2370 is the determined cDNA sequence for clone 62328880 R0398:H11

SEQ ID NO:2371 is the determined cDNA sequence for clone 62296072 R0399:A02

SEQ ID NO:2372 is the determined cDNA sequence for clone 62296073 R0399:A03

SEQ ID NO:2373 is the determined cDNA sequence for clone 62296074 R0399:A05

SEQ ID NO:2374 is the determined cDNA sequence for clone 62296075 R0399:A06

SEQ ID NO:2375 is the determined cDNA sequence for clone 62296076 R0399:A07

SEQ ID NO:2376 is the determined cDNA sequence for clone 62296077 R0399:A08

SEQ ID NO:2377 is the determined cDNA sequence for clone 62296078 R0399:A09

SEQ ID NO:2378 is the determined cDNA sequence for clone 62296079 R0399:A10

SEQ ID NO:2379 is the determined cDNA sequence for clone 62296080 R0399:A11

SEQ ID NO:2380 is the determined cDNA sequence for clone 62296081 R0399:A12

SEQ ID NO:2381 is the determined cDNA sequence for clone 62296082 R0399:B01

SEQ ID NO:2382 is the determined cDNA sequence for clone 62296083 R0399:B02

SEQ ID NO:2383 is the determined cDNA sequence for clone 62296084 R0399:B03

SEQ ID NO:2384 is the determined cDNA sequence for clone 62296085 R0399:B04

SEQ ID NO:2385 is the determined cDNA sequence for clone 62296086 R0399:B05

SEQ ID NO:2386 is the determined cDNA sequence for clone 62296087 R0399:B06

SEQ ID NO:2387 is the determined cDNA sequence for clone 62296088 R0399:B07

SEQ ID NO:2388 is the determined cDNA sequence for clone 62296089 R0399:B08

SEQ ID NO:2389 is the determined cDNA sequence for clone 62296090 R0399:B09

SEQ ID NO:2390 is the determined cDNA sequence for clone 62296091 R0399:B10

SEQ ID NO:2391 is the determined cDNA sequence for clone 62296092 R0399:B11

SEQ ID NO:2392 is the determined cDNA sequence for clone 62296093 R0399:B12

SEQ ID NO:2393 is the determined cDNA sequence for clone 62296094 R0399:C01

SEQ ID NO:2394 is the determined cDNA sequence for clone 62296095 R0399:C02

SEQ ID NO:2395 is the determined cDNA sequence for clone 62296096 R0399:C03

SEQ ID NO:2396 is the determined cDNA sequence for clone 62296097 R0399:C04

SEQ ID NO:2397 is the determined cDNA sequence for clone 62296098 R0399:C05

SEQ ID NO:2398 is the determined cDNA sequence for clone 62296099 R0399:C06

SEQ ID NO:2399 is the determined cDNA sequence for clone 62296100 R0399:C07

SEQ ID NO:2400 is the determined cDNA sequence for clone 62296101 R0399:C08

SEQ ID NO:2401 is the determined cDNA sequence for clone 62296102 R0399:C09

SEQ ID NO:2402 is the determined cDNA sequence for clone 62296103 R0399:C10

SEQ ID NO:2403 is the determined cDNA sequence for clone 62296104 R0399:C11

SEQ ID NO:2404 is the determined cDNA sequence for clone 62296105 R0399:C12

SEQ ID NO:2405 is the determined cDNA sequence for clone 62296107 R0399:D02

SEQ ID NO:2406 is the determined cDNA sequence for clone 62296108 R0399:D03

SEQ ID NO:2407 is the determined cDNA sequence for clone 62296109 R0399:D04

SEQ ID NO:2408 is the determined cDNA sequence for clone 62296110 R0399:D05

SEQ ID NO:2409 is the determined cDNA sequence for clone 62296111 R0399:D06

SEQ ID NO:2410 is the determined cDNA sequence for clone 62296112 R0399:D07

SEQ ID NO:2411 is the determined cDNA sequence for clone 62296113 R0399:D08

SEQ ID NO:2412 is the determined cDNA sequence for clone 62296114 R0399:D09

SEQ ID NO:2413 is the determined cDNA sequence for clone 62296116 R0399:D11

SEQ ID NO:2414 is the determined cDNA sequence for clone 62296117 R0399:D12

SEQ ID NO:2415 is the determined cDNA sequence for clone 62296118 R0399:E01

SEQ ID NO:2416 is the determined cDNA sequence for clone 62296119 R0399:E02

SEQ ID NO:2417 is the determined cDNA sequence for clone 62296120 R0399:E03

SEQ ID NO:2418 is the determined cDNA sequence for clone 62296121 R0399:E04

SEQ ID NO:2419 is the determined cDNA sequence for clone 62296122 R0399:E05

SEQ ID NO:2420 is the determined cDNA sequence for clone 62296123 R0399:E06

SEQ ID NO:2421 is the determined cDNA sequence for clone 62296124 R0399:E07

SEQ ID NO:2422 is the determined cDNA sequence for clone 62296126 R0399:E09

SEQ ID NO:2423 is the determined cDNA sequence for clone 62296128 R0399:E11

SEQ ID NO:2424 is the determined cDNA sequence for clone 62296129 R0399:E12

SEQ ID NO:2425 is the determined cDNA sequence for clone 62296130 R0399:F01

SEQ ID NO:2426 is the determined cDNA sequence for clone 62296131 R0399:F02

SEQ ID NO:2427 is the determined cDNA sequence for clone 62296132 R0399:F03

SEQ ID NO:2428 is the determined cDNA sequence for clone 62296133 R0399:F04

SEQ ID NO:2429 is the determined cDNA sequence for clone 62296134 R0399:F05

SEQ ID NO:2430 is the determined cDNA sequence for clone 62296135 R0399:F06

SEQ ID NO:2431 is the determined cDNA sequence for clone 62296136 R0399:F07

SEQ ID NO:2432 is the determined cDNA sequence for clone 62296137 R0399:F08

SEQ ID NO:2433 is the determined cDNA sequence for clone 62296138 R0399:F09

SEQ ID NO:2434 is the determined cDNA sequence for clone 62296139 R0399:F10

SEQ ID NO:2435 is the determined cDNA sequence for clone 62296140 R0399:F11

SEQ ID NO:2436 is the determined cDNA sequence for clone 62296141 R0399:F12

SEQ ID NO:2437 is the determined cDNA sequence for clone 62296142 R0399:G01

SEQ ID NO:2438 is the determined cDNA sequence for clone 62296143 R0399:G02

SEQ ID NO:2439 is the determined cDNA sequence for clone 62296144 R0399:G03

SEQ ID NO:2440 is the determined cDNA sequence for clone 62296145 R0399:G04

SEQ ID NO:2441 is the determined cDNA sequence for clone 62296146 R0399:G05

SEQ ID NO:2442 is the determined cDNA sequence for clone 62296147 R0399:G06

SEQ ID NO:2443 is the determined cDNA sequence for clone 62296148 R0399:G07

SEQ ID NO:2444 is the determined cDNA sequence for clone 62296149 R0399:G08

SEQ ID NO:2445 is the determined cDNA sequence for clone 62296150 R0399:G09

SEQ ID NO:2446 is the determined cDNA sequence for clone 62296151 R0399:G10

SEQ ID NO:2447 is the determined cDNA sequence for clone 62296152 R0399:G11

SEQ ID NO:2448 is the determined cDNA sequence for clone 62296153 R0399:G12

SEQ ID NO:2449 is the determined cDNA sequence for clone 62296154 R0399:H01

SEQ ID NO:2450 is the determined cDNA sequence for clone 62296155 R0399:H02

SEQ ID NO:2451 is the determined cDNA sequence for clone 62296156 R0399:H03

SEQ ID NO:2452 is the determined cDNA sequence for clone 62296157 R0399:H04

SEQ ID NO:2453 is the determined cDNA sequence for clone 62296158 R0399:H05

SEQ ID NO:2454 is the determined cDNA sequence for clone 62296159 R0399:H06

SEQ ID NO:2455 is the determined cDNA sequence for clone 62296161 R0399:H08

SEQ ID NO:2456 is the determined cDNA sequence for clone 62296162 R0399:H09

SEQ ID NO:2457 is the determined cDNA sequence for clone 62296164 R0399:H11

SEQ ID NO:2458 is the determined cDNA sequence for clone 62295979 R0400:A02

SEQ ID NO:2459 is the determined cDNA sequence for clone 62295981 R0400:A05

SEQ ID NO:2460 is the determined cDNA sequence for clone 62295982 R0400:A06

SEQ ID NO:2461 is the determined cDNA sequence for clone 62295983 R0400:A07

SEQ ID NO:2462 is the determined cDNA sequence for clone 62295984 R0400:A08

SEQ ID NO:2463 is the determined cDNA sequence for clone 62295986 R0400:A10

SEQ ID NO:2464 is the determined cDNA sequence for clone 62295987 R0400:A11

SEQ ID NO:2465 is the determined cDNA sequence for clone 62295988 R0400:A12

SEQ ID NO:2466 is the determined cDNA sequence for clone 62295990 R0400:B02

SEQ ID NO:2467 is the determined cDNA sequence for clone 62295991 R0400:B03

SEQ ID NO:2468 is the determined cDNA sequence for clone 62295992 R0400:B04

SEQ ID NO:2469 is the determined cDNA sequence for clone 62295993 R0400:B05

SEQ ID NO:2470 is the determined cDNA sequence for clone 62295994 R0400:B06

SEQ ID NO:2471 is the determined cDNA sequence for clone 62295995 R0400:B07

SEQ ID NO:2472 is the determined cDNA sequence for clone 62295996 R0400:B08

SEQ ID NO:2473 is the deter-mined cDNA sequence for clone 62295997 R0400:B09

SEQ ID NO:2474 is the determined cDNA sequence for clone 62295998 R0400:B10

SEQ ID NO:2475 is the determined cDNA sequence for clone 62295999 R0400:B11

SEQ ID NO:2476 is the determined cDNA sequence for clone 62296000 R0400:B12

SEQ ID NO:2477 is the determined cDNA sequence for clone 62296001 R0400:C01

SEQ ID NO:2478 is the determined cDNA sequence for clone 62296002 R0400:C02

SEQ ID NO:2479 is the determined cDNA sequence for clone 62296003 R0400:C03

SEQ ID NO:2480 is the determined cDNA sequence for clone 62296005 R0400:C05

SEQ ID NO:2481 is the determined cDNA sequence for clone 62296006 R0400:C06

SEQ ID NO:2482 is the determined cDNA sequence for clone 62296008 R0400:C08

SEQ ID NO:2483 is the determined cDNA sequence for clone 62296009 R0400:C09

SEQ ID NO:2484 is the determined cDNA sequence for clone 62296010 R0400:C10

SEQ ID NO:2485 is the determined cDNA sequence for clone 62296012 R0400:C12

SEQ ID NO:2486 is the determined cDNA sequence for clone 62296013 R0400:D01

SEQ ID NO:2487 is the determined cDNA sequence for clone 62296014 R0400:D02

SEQ ID NO:2488 is the determined cDNA sequence for clone 62296016 R0400:D04

SEQ ID NO:2489 is the determined cDNA sequence for clone 62296017 R0400:D05

SEQ ID NO:2490 is the determined cDNA sequence for clone 62296018 R0400:D06

SEQ ID NO:2491 is the determined cDNA sequence for clone 62296019 R0400:D07

SEQ ID NO:2492 is the determined cDNA sequence for clone 62296020 R0400:D08

SEQ ID NO:2493 is the determined cDNA sequence for clone 62296021 R0400:D09

SEQ ID NO:2494 is the determined cDNA sequence for clone 62296022 R0400:D10

SEQ ID NO:2495 is the determined cDNA sequence for clone 62296023 R0400:D11

SEQ ID NO:2496 is the determined cDNA sequence for clone 62296024 R0400:D12

SEQ ID NO:2497 is the determined cDNA sequence for clone 62296026 R0400:E02

SEQ ID NO:2498 is the determined cDNA sequence for clone 62296028 R0400:E04

SEQ ID NO:2499 is the determined cDNA sequence for clone 62296030 R0400:E06

SEQ ID NO:2500 is the determined cDNA sequence for clone 62296033 R0400:E09

SEQ ID NO:2501 is the determined cDNA sequence for clone 62296034 R0400:E10

SEQ ID NO:2502 is the determined cDNA sequence for clone 62296035 R0400:E11

SEQ ID NO:2503 is the determined cDNA sequence for clone 62296036 R0400:E12

SEQ ID NO:2504 is the determined cDNA sequence for clone 62296037 R0400:F01

SEQ ID NO:2505 is the determined cDNA sequence for clone 62296038 R0400:F02

SEQ ID NO:2506 is the determined cDNA sequence for clone 62296039 R0400:F03

SEQ ID NO:2507 is the determined cDNA sequence for clone 62296040 R0400:F04

SEQ ID NO:2508 is the determined cDNA sequence for clone 62296041 R0400:F05

SEQ ID NO:2509 is the determined cDNA sequence for clone 62296043 R0400:F07

SEQ ID NO:2510 is the determined cDNA sequence for clone 62296045 R0400:F09

SEQ ID NO:2511 is the determined cDNA sequence for clone 62296046 R0400:F10

SEQ ID NO:2512 is the determined cDNA sequence for clone 62296047 R0400:F11

SEQ ID NO:2513 is the determined cDNA sequence for clone 62296048 R0400:F12

SEQ ID NO:2514 is the determined cDNA sequence for clone 62296049 R0400:G01

SEQ ID NO:2515 is the determined cDNA sequence for clone 62296050 R0400:G02

SEQ ID NO:2516 is the determined cDNA sequence for clone 62296051 R0400:G03

SEQ ID NO:2517 is the determined cDNA sequence for clone 62296052 R0400:G04

SEQ ID NO:2518 is the determined cDNA sequence for clone 62296053 R0400:G05

SEQ ID NO:2519 is the determined cDNA sequence for clone 62296054 R0400:G06

SEQ ID NO:2520 is the determined cDNA sequence for clone 62296057 R0400:G09

SEQ ID NO:2521 is the determined cDNA sequence for clone 62296058 R0400:G10

SEQ ID NO:2522 is the determined cDNA sequence for clone 62296059 R0400:G11

SEQ ID NO:2523 is the determined cDNA sequence for clone 62296060 R0400:G12

SEQ ID NO:2524 is the determined cDNA sequence for clone 62296062 R0400:H02

SEQ ID NO:2525 is the determined cDNA sequence for clone 62296064 R0400:H04

SEQ ID NO:2526 is the determined cDNA sequence for clone 62296065 R0400:H05

SEQ ID NO:2527 is the determined cDNA sequence for clone 62296066 R0400:H06

SEQ ID NO:2528 is the determined cDNA sequence for clone 62296067 R0400:H07

SEQ ID NO:2529 is the determined cDNA sequence for clone 62296068 R0400:H08

SEQ ID NO:2530 is the determined cDNA sequence for clone 62296069 R0400:H09

SEQ ID NO:2531 is the determined cDNA sequence for clone 62296070 R0400:H10

SEQ ID NO:2532 is the determined cDNA sequence for clone 62296071 R0400:H11

SEQ ID NO:2533 is the determined cDNA sequence for clone 4C_{—}78.SEQ

SEQ ID NO:2534 is the determined cDNA sequence for clone 4C_{—}28.SEQ

SEQ ID NO:2535 is the determined cDNA sequence for clone 4C_{—}29.SEQ

SEQ ID NO:2536 is the determined cDNA sequence for clone 4C_{—}50.SEQ

SEQ ID NO:2537 is the determined cDNA sequence for clone 4C_{—}16.SEQ

SEQ ID NO:2538 is the determined cDNA sequence for clone 4C_{—}71.SEQ

SEQ ID NO:2539 is the determined cDNA sequence for clone 4C_{—}26.SEQ

SEQ ID NO:2540 is the determined cDNA sequence for clone 4C_{—}30.SEQ

SEQ ID NO:2541 is the determined cDNA sequence for clone 4C_{—}46.SEQ

SEQ ID NO:2542 is the determined cDNA sequence for clone 4C_{—}12.SEQ

SEQ ID NO:2543 is the determined cDNA sequence for clone 4C_{—}61.SEQ

SEQ ID NO:2544 is the determined cDNA sequence for clone 4C_{—}63.SEQ

SEQ ID NO:2545 is the determined cDNA sequence for clone 4C_{—}69.SEQ

SEQ ID NO:2546 is the determined cDNA sequence for clone 4C_{—}34.SEQ

SEQ ID NO:2547 is the determined cDNA sequence for clone 4C_{—}48.SEQ

SEQ ID NO:2548 is the determined cDNA sequence for clone 4C_{—}11.SEQ

SEQ ID NO:2549 is the determined cDNA sequence for clone 4C_{—}44.SEQ

SEQ ID NO:2550 is the determined cDNA sequence for clone 4C_{—}80.SEQ

SEQ ID NO:2551 is the determined cDNA sequence for clone 4C_{—}25.SEQ

SEQ ID NO:2552 is the determined cDNA sequence for clone 4C_{—}40.SEQ

SEQ ID NO:2553 is the determined cDNA sequence for clone 4C_{—}58.SEQ

SEQ ID NO:2554 is the determined cDNA sequence for clone 4C_{—}38.SEQ

SEQ ID NO:2555 is the determined cDNA sequence for clone 4C_{—}76.SEQ

SEQ ID NO:2556 is the determined cDNA sequence for clone 4C_{—}64.SEQ

SEQ ID NO:2557 is the determined cDNA sequence for clone 4C_{—}73.SEQ

SEQ ID NO:2558 is the determined cDNA sequence for clone 4C_{—}52.SEQ

SEQ ID NO:2559 is the determined cDNA sequence for clone 4C_{—}27.SEQ

SEQ ID NO:2560 is the determined cDNA sequence for clone 4C_{—}22.SEQ

SEQ ID NO:2561 is the determined cDNA sequence for clone 4C_{—}10.SEQ

SEQ ID NO:2562 is the determined cDNA sequence for clone 4C_{—}60.SEQ

SEQ ID NO:2563 is the determined cDNA sequence for clone 4C_{—}81.SEQ

SEQ ID NO:2564 is the determined cDNA sequence for clone 4C_{—}41.SEQ

SEQ ID NO:2565 is the determined cDNA sequence for clone 4C_{—}47.SEQ

SEQ ID NO:2566 is the determined cDNA sequence for clone 4C_{—}21.SEQ

SEQ ID NO:2567 is the determined cDNA sequence for clone 4C_{—}65.SEQ

SEQ ID NO:2568 is the determined cDNA sequence for clone 4C_{—}74.SEQ

SEQ ID NO:2569 is the determined cDNA sequence for clone 4C_{—}5.SEQ

SEQ ID NO:2570 is the determined cDNA sequence for clone 4C_{—}51.SEQ

SEQ ID NO:2571 is the determined cDNA sequence for clone 4C75.SEQ

SEQ ID NO:2572 is the determined cDNA sequence for clone 4C_{—}6.SEQ

SEQ ID NO:2573 is the determined cDNA sequence for clone 4C_{—}35.SEQ

SEQ ID NO:2574 is the determined cDNA sequence for clone 4C_{—}57.SEQ

SEQ ID NO:2575 is the determined cDNA sequence for clone 4C_{—}33.SEQ

SEQ ID NO:2576 is the determined cDNA sequence for clone 4C_{—}9.SEQ

SEQ ID NO:2577 is the determined cDNA sequence for clone 4C_{—}72.SEQ

SEQ ID NO:2578 is the determined cDNA sequence for clone 4C_{—}8.SEQ

SEQ ID NO:2579 is the determined cDNA sequence for clone 4C_{—}68.SEQ

SEQ ID NO:2580 is the determined cDNA sequence for clone 4C_{—}54.SEQ

SEQ ID NO:2581 is the determined cDNA sequence for clone 4C_{—}32.SEQ

SEQ ID NO:2582 is the determined cDNA sequence for clone 4C_{—}82.SEQ

SEQ ID NO:2583 is the determined cDNA sequence for clone 4C_{—}4.SEQ

SEQ ID NO:2584 is the determined cDNA sequence for clone 4C_{—}18.SEQ

SEQ ID NO:2585 is the determined cDNA sequence for clone 4C_{—}86.SEQ

SEQ ID NO:2586 is the determined full-length cDNA sequence for clone C1466P that shows similarity to human TOP2A

SEQ ID NO:2587 is the determined full-length cDNA sequence for clone C1465P that shows similarity to human CDX1

SEQ ID NO:2588 is the determined full-length cDNA sequence for clone C1446P that shows similarity to FLJ21522

SEQ ID NO:2589 is the determined full-length cDNA sequence for clone C1444P that shows similarity to Claudin2

SEQ ID NO:2590 is the full-length protein sequence for clone C1444P, encoded by the cDNA sequence set forth in SEQ ID NO:2589

SEQ ID NO:2591 is the full-length protein sequence for clone C1446P encoded by the cDNA sequence set forth in SEQ ID NO:2588

SEQ ID NO:2592 is the full-length protein sequence for clone C1465P encoded by the cDNA sequence set forth in SEQ ID NO:2587

SEQ ID NO:2593 is the full-length protein sequence for clone C1466P encoded by the cDNA sequence set forth in SEQ ID NO:2585

SEQ ID NO:2594 is the determined cDNA sequence for clone R0363:E01

SEQ ID NO:2595 is the determined cDNA sequence for clone R0364:B08

SEQ ID NO:2596 is the determined cDNA sequence for clone R0366:G06

SEQ ID NO:2597 is the determined cDNA sequence for clone R0366:B10

SEQ ID NO:2598 is the determined cDNA sequence for clone R0369:H04

SEQ ID NO:2599 is the determined cDNA sequence for clone R0370:B06

SEQ ID NO:2600 is the determined cDNA sequence for clone R0372:A11

SEQ ID NO:2601 is the determined cDNA sequence for clone R0373:A02

SEQ ID NO:2602 is the determined full-length cDNA sequence for clone C638S_hiwi

SEQ ID NO:2603 is the full-length protein sequence for clone C638S_hiwi_ORF encoded by the cDNA sequence set forth in SEQ ID NO:2602

SEQ ID NO:2604 is the determined cDNA sequence for clone R0362:E12

SEQ ID NO:2605 is the determined full-length cDNA sequence for clone R0400:B8 that shows similarity to SCYA20

SEQ ID NO:2606 is the full-length protein sequence for SCYA20 encoded by the cDNA set forth in SEQ ID NO:2605

### DETAILED DESCRIPTION OF THE INVENTION

The present invention is directed generally to compositions and their use in the therapy and diagnosis of cancer, particularly colon cancer. As described further below, illustrative compositions of the present invention include, but are not restricted to, polypeptides, particularly immunogenic polypeptides, polynucleotides encoding such polypeptides, antibodies and other binding agents, antigen presenting cells (APCs) and immune system cells (e.g., T cells).

The practice of the present invention will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Sambrook, et al. Molecular Cloning: A Laboratory Manual (2nd Edition, 1989); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984).

All publications, patents and patent applications cited herein, whether supra or infra, are hereby incorporated by reference in their entirety.

As used in this specification and the appended claims, the singular forms “a,” “an” and “the” include plural references unless the content clearly dictates otherwise.

Polypeptide Compositions

As used herein, the term “polypeptide” “is used in its conventional meaning, i.e., as a sequence of amino acids. The polypeptides are not limited to a specific length of the product; thus, peptides, oligopeptides, and proteins are included within the definition of polypeptide, and such terms may be used interchangeably herein unless specifically indicated otherwise. This term also does not refer to or exclude post-expression modifications of the polypeptide, for example, glycosylations, acetylations, phosphorylations and the like, as well as other modifications known in the art, both naturally occurring and non-naturally occurring. A polypeptide may be an entire protein, or a subsequence thereof. Particular polypeptides of interest in the context of this invention are amino acid subsequences comprising epitopes, i.e., antigenic determinants substantially responsible for the immunogenic properties of a polypeptide and being capable of evoking an immune response.

Particularly illustrative polypeptides of the present invention comprise those encoded by a polynucleotide sequence set forth in any one of SEQ ID NO:1-2589, 2594-2602, and 2604-2605, or a sequence that hybridizes under moderately stringent conditions, or, alternatively, under highly stringent conditions, to a polynucleotide sequence set forth in any one of SEQ ID NO:1-2589, 2594-2602, and 2604-2605. Certain other illustrative polypeptides of the invention comprise amino acid sequences as set forth in any one of SEQ ID NOs:2590-2593, 2603, and 2606.

The polypeptides of the present invention are sometimes herein referred to as colon tumor proteins or colon tumor polypeptides, as an indication that their identification has been based at least in part upon their increased levels of expression in colon tumor samples. Thus, a “colon tumor polypeptide” or “colon tumor protein,” refers generally to a polypeptide sequence of the present invention, or a polynucleotide sequence encoding such a polypeptide, that is expressed in a substantial proportion of colon tumor samples, for example preferably greater than about 20%, more preferably greater than about 30%, and most preferably greater than about 50% or more of colon tumor samples tested, at a level that is at least two fold, and preferably at least five fold, greater than the level of expression in normal tissues, as determined using a representative assay provided herein. A colon tumor polypeptide sequence of the invention, based upon its increased level of expression in tumor cells, has particular utility both as a diagnostic marker as well as a therapeutic target, as further described below.

In certain preferred embodiments, the polypeptides of the invention are immunogenic, i.e., they react detectably within an immunoassay (such as an ELISA or T-cell stimulation assay) with antisera and/or T-cells from a patient with colon cancer. Screening for immunogenic activity can be performed using techniques well known to the skilled artisan. For example, such screens can be performed using methods such as those described in Harlow and Lane, Antibodies: A Laboratory Manual, Cold Spring Harbor Laboratory, 1988. In one illustrative example, a polypeptide may be immobilized on a solid support and contacted with patient sera to allow binding of antibodies within the sera to the immobilized polypeptide. Unbound sera may then be removed and bound antibodies detected using, for example, ¹²⁵I-labeled Protein A.

As would be recognized by the skilled artisan, immunogenic portions of the polypeptides disclosed herein are also encompassed by the present invention. An “immunogenic portion,” as used herein, is a fragment of an immunogenic polypeptide of the invention that itself is immunologically reactive (i.e., specifically binds) with the B-cells and/or T-cell surface antigen receptors that recognize the polypeptide. Immunogenic portions may generally be identified using well known techniques, such as those summarized in Paul, Fundamental Immunology, 3rd ed., 243-247 (Raven Press, 1993) and references cited therein. Such techniques include screening polypeptides for the ability to react with antigen-specific antibodies, antisera and/or T-cell lines or clones. As used herein, antisera and antibodies are “antigen-specific” if they specifically bind to an antigen (i.e., they react with the protein in an ELISA or other immunoassay, and do not react detectably with unrelated proteins). Such antisera and antibodies may be prepared as described herein, and using well-known techniques.

In one preferred embodiment, an immunogenic portion of a polypeptide of the present invention is a portion that reacts with antisera and/or T-cells at a level that is not substantially less than the reactivity of the full-length polypeptide (e.g., in an ELISA and/or T-cell reactivity assay). Preferably, the level of immunogenic activity of the immunogenic portion is at least about 50%, preferably at least about 70% and most preferably greater than about 90% of the immunogenicity for the full-length polypeptide. In some instances, preferred immunogenic portions will be identified that have a level of immunogenic activity greater than that of the corresponding full-length polypeptide, e.g., having greater than about 100% or 150% or more immunogenic activity.

In certain other embodiments, illustrative immunogenic portions may include peptides in which an N-terminal leader sequence and/or transmembrane domain have been deleted. Other illustrative immunogenic portions will contain a small N- and/or C-terminal deletion (e.g., 1-30 amino acids, preferably 5-15 amino acids), relative to the mature protein.

In another embodiment, a polypeptide composition of the invention may also comprise one or more polypeptides that are immunologically reactive with T cells and/or antibodies generated against a polypeptide of the invention, particularly a polypeptide having an amino acid sequence disclosed herein, or to an immunogenic fragment or variant thereof.

In another embodiment of the invention, polypeptides are provided that comprise one or more polypeptides that are capable of eliciting T cells and/or antibodies that are immunologically reactive with one or more polypeptides described herein, or one or more polypeptides encoded by contiguous nucleic acid sequences contained in the polynucleotide sequences disclosed herein, or immunogenic fragments or variants thereof, or to one or more nucleic acid sequences which hybridize to one or more of these sequences under conditions of moderate to high stringency.

The present invention, in another aspect, provides polypeptide fragments comprising at least about 5, 10, 15, 20, 25, 50, or 100 contiguous amino acids, or more, including all intermediate lengths, of polypeptide compositions set forth herein, such as those set forth in SEQ ID NOs:2590-2593, 2603, and 2606, or those encoded by a polynucleotide sequence set forth in a sequence of SEQ ID NO:1-2589, 2594-2602, and 2604-2605.

In another aspect, the present invention provides variants of the polypeptide compositions described herein. Polypeptide variants generally encompassed by the present invention will typically exhibit at least about 70%, 75%, 80%, 85%, 90%, 91%, 92%, 93%, 94%, 95%, 96%, 97%, 98%, or 99% or more identity (determined as described below), along its length, to a polypeptide sequences set forth herein.

In one preferred embodiment, the polypeptide fragments and variants provide by the present invention are immunologically reactive with an antibody and/or T-cell that reacts with a full-length polypeptide specifically set for the herein.

In another preferred embodiment, the polypeptide fragments and variants provided by the present invention exhibit a level of immunogenic activity of at least about 50%, preferably at least about 70%, and most preferably at least about 90% or more of that exhibited by a full-length polypeptide sequence specifically set forth herein.

A polypeptide “variant,” as the term is used herein, is a polypeptide that typically differs from a polypeptide specifically disclosed herein in one or more substitutions, deletions, additions and/or insertions. Such variants may be naturally occurring or may be synthetically generated, for example, by modifying one or more of the above polypeptide sequences of the invention and evaluating their immunogenic activity as described herein and/or using any of a number of techniques well known in the art.

For example, certain illustrative variants of the polypeptides of the invention include those in which one or more portions, such as an N-terminal leader sequence or transmembrane domain, have been removed. Other illustrative variants include variants in which a small portion (e.g., 1-30 amino acids, preferably 5-15 amino acids) has been removed from the N- and/or C-terminal of the mature protein.

In many instances, a variant will contain conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. As described above, modifications may be made in the structure of the polynucleotides and polypeptides of the present invention and still obtain a functional molecule that encodes a variant or derivative polypeptide with desirable characteristics, e.g., with immunogenic characteristics. When it is desired to alter the amino acid sequence of a polypeptide to create an equivalent, or even an improved, immunogenic variant or portion of a polypeptide of the invention, one skilled in the art will typically change one or more of the codons of the encoding DNA sequence according to Table 1.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences which encode said peptides without appreciable loss of their biological utility or activity.

TABLE 1

| Amino Acids | | | Codons | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Giu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Giutamine | Gin | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

In making such changes, the hydropathic index of amino acids may be considered. The importance of the hydropathic amino acid index in conferring interactive biologic function on a protein is generally understood in the art (Kyte and Doolittle, 1982, incorporated herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity. U.S. Pat. No. 4,554,101 (specifically incorporated herein by reference in its entirety), states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

In addition, any polynucleotide may be further modified to increase stability in vivo. Possible modifications include, but are not limited to, the addition of flanking sequences at the 5′ and/or 3′ ends; the use of phosphorothioate or 2′ O-methyl rather than phosphodiesterase linkages in the backbone; and/or the inclusion of nontraditional bases such as inosine, queosine and wybutosine, as well as acetyl- methyl-, thio- and other modified forms of adenine, cytidine, guanine, thymine and uridine.

Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

As noted above, polypeptides may comprise a signal (or leader) sequence at the N-terminal end of the protein, which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

When comparing polypeptide sequences, two sequences are said to be “identical” if the sequence of amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington DC Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information. For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one preferred approach, the “percentage of sequence identity” is determined by comparing two optimally aligned sequences over a window of comparison of at least 20 positions, wherein the portion of the polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the reference sequence (i.e., the window size) and multiplying the results by 100 to yield the percentage of sequence identity.

Within other illustrative embodiments, a polypeptide may be a fusion polypeptide that comprises multiple polypeptides as described herein, or that comprises at least one polypeptide as described herein and an unrelated sequence, such as a known tumor protein. A fusion partner may, for example, assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the polypeptide or to enable the polypeptide to be targeted to desired intracellular compartments. Still further fusion partners include affinity tags, which facilitate purification of the polypeptide.

Fusion polypeptides may generally be prepared using standard techniques, including chemical conjugation. Preferably, a fusion polypeptide is expressed as a recombinant polypeptide, allowing the production of increased levels, relative to a non-fused polypeptide, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion polypeptide that retains the biological activity of both component polypeptides.

A peptide linker sequence may be employed to separate the first and second polypeptide components by a distance sufficient to ensure that each polypeptide folds into its secondary and tertiary structures. Such a peptide linker sequence is incorporated into the fusion polypeptide using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

The fusion polypeptide can comprise a polypeptide as described herein together with an unrelated immunogenic protein, such as an immunogenic protein capable of eliciting a recall response. Examples of such proteins include tetanus, tuberculosis and hepatitis proteins (see, for example, Stoute et al. New Engl. J. Med., 336:86-91, 1997).

In one preferred embodiment, the immunological fusion partner is derived from a Mycobacterium sp., such as a Mycobacterium tuberculosis-derived Ra12 fragment. Ra12 compositions and methods for their use in enhancing the expression and/or immunogenicity of heterologous polynucleotide/polypeptide sequences is described in U.S. Patent Application No. 60/158,585, the disclosure of which is incorporated herein by reference in its entirety. Briefly, Ra12 refers to a polynucleotide region that is a subsequence of a Mycobacterium tuberculosis MTB32A nucleic acid. MTB32A is a serine protease of 32 KD molecular weight encoded by a gene in virulent and avirulent strains of M. tuberculosis. The nucleotide sequence and amino acid sequence of MTB32A have been described (for example, U.S. Patent Application No. 60/158,585; see also, Skeiky et al., Infection and Immun. (1999) 67:3998-4007, incorporated herein by reference). C-terminal fragments of the MTB32A coding sequence express at high levels and remain as a soluble polypeptides throughout the purification process. Moreover, Ra12 may enhance the immunogenicity of heterologous immunogenic polypeptides with which it is fused. One preferred Ra12 fusion polypeptide comprises a 14 KD C-terminal fragment corresponding to amino acid residues 192 to 323 of MTB32A. Other preferred Ra12 polynucleotides generally comprise at least about 15 consecutive nucleotides, at least about 30 nucleotides, at least about 60 nucleotides, at least about 100 nucleotides, at least about 200 nucleotides, or at least about 300 nucleotides that encode a portion of a Ra12 polypeptide. Ra12 polynucleotides may comprise a native sequence (i.e., an endogenous sequence that encodes a Ra12 polypeptide or a portion thereof) or may comprise a variant of such a sequence. Ra12 polynucleotide variants may contain one or more substitutions, additions, deletions and/or insertions such that the biological activity of the encoded fusion polypeptide is not substantially diminished, relative to a fusion polypeptide comprising a native Ra12 polypeptide. Variants preferably exhibit at least about 70% identity, more preferably at least about 80% identity and most preferably at least about 90% identity to a polynucleotide sequence that encodes a native Ra12 polypeptide or a portion thereof.

Within other preferred embodiments, an immunological fusion partner is derived from protein D, a surface protein of the gram-negative bacterium Haemophilus influenza B (WO 91/18926). Preferably, a protein D derivative comprises approximately the first third of the protein (e.g., the first N-terminal 100-110 amino acids), and a protein D derivative may be lipidated. Within certain preferred embodiments, the first 109 residues of a Lipoprotein D fusion partner is included on the N-terminus to provide the polypeptide with additional exogenous T-cell epitopes and to increase the expression level in E. coli (thus functioning as an expression enhancer). The lipid tail ensures optimal presentation of the antigen to antigen presenting cells. Other fusion partners include the non-structural protein from influenzae virus, NS1 (hemaglutinin). Typically, the N-terminal 81 amino acids are used, although different fragments that include T-helper epitopes may be used.

In another embodiment, the immunological fusion partner is the protein known as LYTA, or a portion thereof (preferably a C-terminal portion). LYTA is derived from Streptococcus pneumoniae, which synthesizes an N-acetyl-L-alanine amidase known as amidase LYTA (encoded by the LytA gene; Gene 43:265-292, 1986). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus has been described (see Biotechnology 10:795-798, 1992). Within a preferred embodiment, a repeat portion of LYTA may be incorporated into a fusion polypeptide. A repeat portion is found in the C-terminal region starting at residue 178. A particularly preferred repeat portion incorporates residues 188-305.

Yet another illustrative embodiment involves fusion polypeptides, and the polynucleotides encoding them, wherein the fusion partner comprises a targeting signal capable of directing a polypeptide to the endosomal/lysosomal compartment, as described in U.S. Pat. No. 5,633,234. An immunogenic polypeptide of the invention, when fused with this targeting signal, will associate more efficiently with MHC class II molecules and thereby provide enhanced in vivo stimulation of CD4⁺ T-cells specific for the polypeptide.

Polypeptides of the invention are prepared using any of a variety of well known synthetic and/or recombinant techniques, the latter of which are further described below. Polypeptides, portions and other variants generally less than about 150 amino acids can be generated by synthetic means, using techniques well known to those of ordinary skill in the art. In one illustrative example, such polypeptides are synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Perkin Elmer/Applied BioSystems Division (Foster City, Calif.), and may be operated according to the manufacturer's instructions.

In general, polypeptide compositions (including fusion polypeptides) of the invention are isolated. An “isolated” polypeptide is one that is removed from its original environment. For example, a naturally-occurring protein or polypeptide is isolated if it is separated from some or all of the coexisting materials in the natural system. Preferably, such polypeptides are also purified, e.g., are at least about 90% pure, more preferably at least about 95% pure and most preferably at least about 99% pure.

Polynucleotide Compositions

The present invention, in other aspects, provides polynucleotide compositions. The terms “DNA” and “polynucleotide” are used essentially interchangeably herein to refer to a DNA molecule that has been isolated free of total genomic DNA of a particular species. “Isolated,” as used herein, means that a polynucleotide is substantially away from other coding sequences, and that the DNA molecule does not contain large portions of unrelated coding DNA, such as large chromosomal fragments or other functional genes or polypeptide coding regions. Of course, this refers to the DNA molecule as originally isolated, and does not exclude genes or coding regions later added to the segment by the hand of man.

As will be understood by those skilled in the art, the polynucleotide compositions of this invention can include genomic sequences, extra-genomic and plasmid-encoded sequences and smaller engineered gene segments that express, or may be adapted to express, proteins, polypeptides, peptides and the like. Such segments may be naturally isolated, or modified synthetically by the hand of man.

As will be also recognized by the skilled artisan, polynucleotides of the invention may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes a polypeptide/protein of the invention or a portion thereof) or may comprise a sequence that encodes a variant or derivative, preferably and immunogenic variant or derivative, of such a sequence.

Therefore, according to another aspect of the present invention, polynucleotide compositions are provided that comprise some or all of a polynucleotide sequence set forth in any one of SEQ ID NO:1-2589, 2594-2602, and 2604-2605, complements of a polynucleotide sequence set forth in any one of SEQ ID NO:1-2589, 2594-2602, and 2604-2605, and degenerate variants of a polynucleotide sequence set forth in any one of SEQ ID NO:1-2589, 2594-2602, and 2604-2605. In certain preferred embodiments, the polynucleotide sequences set forth herein encode immunogenic polypeptides, as described above.

In other related embodiments, the present invention provides polynucleotide variants having substantial identity to the sequences disclosed herein in SEQ ID NOs:1-2589, 2594-2602, and 2604-2605, for example those comprising at least 70% sequence identity, preferably at least 75%, 80%, 85%, 90%, 95%, 96%, 97%, 98%, or 99% or higher, sequence identity compared to a polynucleotide sequence of this invention using the methods described herein, (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like.

Typically, polynucleotide variants will contain one or more substitutions, additions, deletions and/or insertions, preferably such that the immunogenicity of the polypeptide encoded by the variant polynucleotide is not substantially diminished relative to a polypeptide encoded by a polynucleotide sequence specifically set forth herein). The term “variants” should also be understood to encompasses homologous genes of xenogenic origin.

In additional embodiments, the present invention provides polynucleotide fragments comprising various lengths of contiguous stretches of sequence identical to or complementary to one or more of the sequences disclosed herein. For example, polynucleotides are provided by this invention that comprise at least about 10, 15, 20, 30, 40, 50, 75, 100, 150, 200, 300, 400, 500 or 1000 or more contiguous nucleotides of one or more of the sequences disclosed herein as well as all intermediate lengths there between. It will be readily understood that “intermediate lengths”, in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like.

In another embodiment of the invention, polynucleotide compositions are provided that are capable of hybridizing under moderate to high stringency conditions to a polynucleotide sequence provided herein, or a fragment thereof, or a complementary sequence thereof. Hybridization techniques are well known in the art of molecular biology. For purposes of illustration, suitable moderately stringent conditions for testing the hybridization of a polynucleotide of this invention with other polynucleotides include prewashing in a solution of 5× SSC, 0.5% SDS, 1.0 mM EDTA (pH 8.0); hybridizing at 50° C.-60° C., 5× SSC, overnight; followed by washing twice at 65° C. for 20 minutes with each of 2×, 0.5× and 0.2× SSC containing 0.1% SDS. One skilled in the art will understand that the stringency of hybridization can be readily manipulated, such as by altering the salt content of the hybridization solution and/or the temperature at which the hybridization is performed. For example, in another embodiment, suitable highly stringent hybridization conditions include those described above, with the exception that the temperature of hybridization is increased, e.g., to 60-65° C. or 65-70° C.

In certain preferred embodiments, the polynucleotides described above, e.g., polynucleotide variants, fragments and hybridizing sequences, encode polypeptides that are immunologically cross-reactive with a polypeptide sequence specifically set forth herein. In other preferred embodiments, such polynucleotides encode polypeptides that have a level of immunogenic activity of at least about 50%, preferably at least about 70%, and more preferably at least about 90% of that for a polypeptide sequence specifically set forth herein.

The polynucleotides of the present invention, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative polynucleotide segments with total lengths of about 10,000, about 5000, about 3000, about 2,000, about 1,000, about 500, about 200, about 100, about 50 base pairs in length, and the like, (including all intermediate lengths) are contemplated to be useful in many implementations of this invention.

When comparing polynucleotide sequences, two sequences are said to be “identical” if the sequence of nucleotides in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington DC Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides of the invention. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=-−4 and a comparison of both strands.

Preferably, the “percentage of sequence identity” is determined by comparing two optimally aligned sequences over a window of comparison of at least 20 positions, wherein the portion of the polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases occurs in both sequences to yield the number of matched positions, dividing the number of matched positions by the total number of positions in the reference sequence (i.e., the window size) and multiplying the results by 100 to yield the percentage of sequence identity.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Therefore, in another embodiment of the invention, a mutagenesis approach, such as site-specific mutagenesis, is employed for the preparation of immunogenic variants and/or derivatives of the polypeptides described herein. By this approach, specific modifications in a polypeptide sequence can be made through mutagenesis of the underlying polynucleotides that encode them. These techniques provides a straightforward approach to prepare and test sequence variants, for example, incorporating one or more of the foregoing considerations, by introducing one or more nucleotide sequence changes into the polynucleotide.

Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or otherwise change the properties of the polynucleotide itself, and/or alter the properties, activity, composition, stability, or primary sequence of the encoded polypeptide.

In certain embodiments of the present invention, the inventors contemplate the mutagenesis of the disclosed polynucleotide sequences to alter one or more properties of the encoded polypeptide, such as the immunogenicity of a polypeptide vaccine. The techniques of site-specific mutagenesis are well-known in the art, and are widely used to create variants of both polypeptides and polynucleotides. For example, site-specific mutagenesis is often used to alter a specific portion of a DNA molecule. In such embodiments, a primer comprising typically about 14 to about 25 nucleotides or so in length is employed, with about 5 to about 10 residues on both sides of the junction of the sequence being altered.

As will be appreciated by those of skill in the art, site-specific mutagenesis techniques have often employed a phage vector that exists in both a single stranded and double stranded form. Typical vectors useful in site-directed mutagenesis include vectors such as the M13 phage. These phage are readily commercially-available and their use is generally well-known to those skilled in the art. Double-stranded plasmids are also routinely employed in site directed mutagenesis that eliminates the step of transferring the gene of interest from a plasmid to a phage.

In general, site-directed mutagenesis in accordance herewith is performed by first obtaining a single-stranded vector or melting apart of two strands of a double-stranded vector that includes within its sequence a DNA sequence that encodes the desired peptide. An oligonucleotide primer bearing the desired mutated sequence is prepared, generally synthetically. This primer is then annealed with the single-stranded vector, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement.

The preparation of sequence variants of the selected peptide-encoding DNA segments using site-directed mutagenesis provides a means of producing potentially useful species and is not meant to be limiting as there are other ways in which sequence variants of peptides and the DNA sequences encoding them may be obtained. For example, recombinant vectors encoding the desired peptide sequence may be treated with mutagenic agents, such as hydroxylamine, to obtain sequence variants. Specific details regarding these methods and protocols are found in the teachings of Maloy et al., 1994; Segal, 1976; Prokop and Bajpai, 1991; Kuby, 1994; and Maniatis et al., 1982, each incorporated herein by reference, for that purpose.

As used herein, the term “oligonucleotide directed mutagenesis procedure” refers to template-dependent processes and vector-mediated propagation which result in an increase in the concentration of a specific nucleic acid molecule relative to its initial concentration, or in an increase in the concentration of a detectable signal, such as amplification. As used herein, the term “oligonucleotide directed mutagenesis procedure” is intended to refer to a process that involves the template-dependent extension of a primer molecule. The term template dependent process refers to nucleic acid synthesis of an RNA or a DNA molecule wherein the sequence of the newly synthesized strand of nucleic acid is dictated by the well-known rules of complementary base pairing (see, for example, Watson, 1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224, specifically incorporated herein by reference in its entirety.

In another approach for the production of polypeptide variants of the present invention, recursive sequence recombination, as described in U.S. Pat. No. 5,837,458, may be employed. In this approach, iterative cycles of recombination and screening or selection are performed to “evolve” individual polynucleotide variants of the invention having, for example, enhanced immunogenic activity.

In other embodiments of the present invention, the polynucleotide sequences provided herein can be advantageously used as probes or primers for nucleic acid hybridization. As such, it is contemplated that nucleic acid segments that comprise a sequence region of at least about 15 nucleotide long contiguous sequence that has the same sequence as, or is complementary to, a 15 nucleotide long contiguous sequence disclosed herein will find particular utility. Longer contiguous identical or complementary sequences, e.g., those of about 20, 30, 40, 50, 100, 200, 500, 1000 (including all intermediate lengths) and even up to full length sequences will also be of use in certain embodiments.

The ability of such nucleic acid probes to specifically hybridize to a sequence of interest will enable them to be of use in detecting the presence of complementary sequences in a given sample. However, other uses are also envisioned, such as the use of the sequence information for the preparation of mutant species primers, or primers for use in preparing other genetic constructions.

Polynucleotide molecules having sequence regions consisting of contiguous nucleotide stretches of 10-14, 15-20, 30, 50, or even of 100-200 nucleotides or so (including intermediate lengths as well), identical or complementary to a polynucleotide sequence disclosed herein, are particularly contemplated as hybridization probes for use in, e.g., Southern and Northern blotting. This would allow a gene product, or fragment thereof, to be analyzed, both in diverse cell types and also in various bacterial cells. The total size of fragment, as well as the size of the complementary stretch(es), will ultimately depend on the intended use or application of the particular nucleic acid segment. Smaller fragments will generally find use in hybridization embodiments, wherein the length of the contiguous complementary region may be varied, such as between about 15 and about 100 nucleotides, but larger contiguous complementarity stretches may be used, according to the length complementary sequences one wishes to detect.

The use of a hybridization probe of about 15-25 nucleotides in length allows the formation of a duplex molecule that is both stable and selective. Molecules having contiguous complementary sequences over stretches greater than 15 bases in length are generally preferred, though, in order to increase stability and selectivity of the hybrid, and thereby improve the quality and degree of specific hybrid molecules obtained. One will generally prefer to design nucleic acid molecules having gene-complementary stretches of 15 to 25 contiguous nucleotides, or even longer where desired.

Hybridization probes may be selected from any portion of any of the sequences disclosed herein. All that is required is to review the sequences set forth herein, or to any continuous portion of the sequences, from about 15-25 nucleotides in length up to and including the full length sequence, that one wishes to utilize as a probe or primer. The choice of probe and primer sequences may be governed by various factors. For example, one may wish to employ primers from towards the termini of the total sequence.

Small polynucleotide segments or fragments may be readily prepared by, for example, directly synthesizing the fragment by chemical means, as is commonly practiced using an automated oligonucleotide synthesizer. Also, fragments may be obtained by application of nucleic acid reproduction technology, such as the PCRTM technology of U.S. Pat. No. 4,683,202 (incorporated herein by reference), by introducing selected sequences into recombinant vectors for recombinant production, and by other recombinant DNA techniques generally known to those of skill in the art of molecular biology.

The nucleotide sequences of the invention may be used for their ability to selectively form duplex molecules with complementary stretches of the entire gene or gene fragments of interest. Depending on the application envisioned, one will typically desire to employ varying conditions of hybridization to achieve varying degrees of selectivity of probe towards target sequence. For applications requiring high selectivity, one will typically desire to employ relatively stringent conditions to form the hybrids, e.g., one will select relatively low salt and/or high temperature conditions, such as provided by a salt concentration of from about 0.02 M to about 0.15 M salt at temperatures of from about 50° C. to about 70° C. Such selective conditions tolerate little, if any, mismatch between the probe and the template or target strand, and would be particularly suitable for isolating related sequences.

Of course, for some applications, for example, where one desires to prepare mutants employing a mutant primer strand hybridized to an underlying template, less stringent (reduced stringency) hybridization conditions will typically be needed in order to allow formation of the heteroduplex. In these circumstances, one may desire to employ salt conditions such as those of from about 0.15 M to about 0.9 M salt, at temperatures ranging from about 20° C. to about 55° C. Cross-hybridizing species can thereby be readily identified as positively hybridizing signals with respect to control hybridizations. In any case, it is generally appreciated that conditions can be rendered more stringent by the addition of increasing amounts of formamide, which serves to destabilize the hybrid duplex in the same manner as increased temperature. Thus, hybridization conditions can be readily manipulated, and thus will generally be a method of choice depending on the desired results.

According to another embodiment of the present invention, polynucleotide compositions comprising antisense oligonucleotides are provided. Antisense oligonucleotides have been demonstrated to be effective and targeted inhibitors of protein synthesis, and, consequently, provide a therapeutic approach by which a disease can be treated by inhibiting the synthesis of proteins that contribute to the disease. The efficacy of antisense oligonucleotides for inhibiting protein synthesis is well established. For example, the synthesis of polygalactauronase and the muscarine type 2 acetylcholine receptor are inhibited by antisense oligonucleotides directed to their respective mRNA sequences (U.S. Pat. No. 5,739,119 and U.S. Pat. No. 5,759,829). Further, examples of antisense inhibition have been demonstrated with the nuclear protein cyclin, the multiple drug resistance gene (MDG1), ICAM-1, E-selectin, STK-1, striatal GABA_{A} receptor and human EGF (Jaskulski et al., Science. 1988 Jun 10;240(4858):1544-6; Vasanthakumar and Ahmed, Cancer Commun. 1989;1(4):225-32; Peris et al., Brain Res Mol Brain Res. 1998 Jun 15;57(2):310-20; U.S. Pat. No. 5,801,154; U.S. Pat. No. 5,789,573; U.S. Pat. No. 5,718,709 and U.S. Pat. No. 5,610,288). Antisense constructs have also been described that inhibit and can be used to treat a variety of abnormal cellular proliferations, e.g. cancer (U.S. Pat. No. 5,747,470; U.S. Pat. No. 5,591,317 and U.S. Pat. No. 5,783,683).

Therefore, in certain embodiments, the present invention provides oligonucleotide sequences that comprise all, or a portion of, any sequence that is capable of specifically binding to polynucleotide sequence described herein, or a complement thereof. In one embodiment, the antisense oligonuclotides comprise DNA or derivatives thereof. In another embodiment, the oligonucleotides comprise RNA or derivatives thereof. In a third embodiment, the oligonucleotides are modified DNAs comprising a phosphorothioated modified backbone. In a fourth embodiment, the oligonucleotide sequences comprise peptide nucleic acids or derivatives thereof. In each case, preferred compositions comprise a sequence region that is complementary, and more preferably substantially-complementary, and even more preferably, completely complementary to one or more portions of polynucleotides disclosed herein. Selection of antisense compositions specific for a given gene sequence is based upon analysis of the chosen target sequence and determination of secondary structure, Tₘ, binding energy, and relative stability. Antisense compositions may be selected based upon their relative inability to form dimers, hairpins, or other secondary structures that would reduce or prohibit specific binding to the target mRNA in a host cell. Highly preferred target regions of the mRNA, are those which are at or near the AUG translation initiation codon, and those sequences which are substantially complementary to 5′ regions of the mRNA. These secondary structure analyses and target site selection considerations can be performed, for example, using v.4 of the OLIGO primer analysis software and/or the BLASTN 2.0.5 algorithm software (Altschul et al., Nucleic Acids Res. 1997, 25(17):3389-402).

The use of an antisense delivery method employing a short peptide vector, termed MPG (27 residues), is also contemplated. The MPG peptide contains a hydrophobic domain derived from the fusion sequence of HIV gp41 and a hydrophilic domain from the nuclear localization sequence of SV40 T-antigen (Morris et al., Nucleic Acids Res. 1997 Jul 15;25(14):2730-6). It has been demonstrated that several molecules of the MPG peptide coat the antisense oligonucleotides and can be delivered into cultured mammalian cells in less than 1 hour with relatively high efficiency (90%). Further, the interaction with MPG strongly increases both the stability of the oligonucleotide to nuclease and the ability to cross the plasma membrane.

According to another embodiment of the invention, the polynucleotide compositions described herein are used in the design and preparation of ribozyme molecules for inhibiting expression of the tumor polypeptides and proteins of the present invention in tumor cells. Ribozymes are RNA-protein complexes that cleave nucleic acids in a site-specific fashion. Ribozymes have specific catalytic domains that possess endonuclease activity (Kim and Cech, Proc Natl Acad Sci U S A. 1987 Dec;84(24):8788-92; Forster and Symons, Cell. 1987 Apr 24;49(2):211-20). For example, a large number of ribozymes accelerate phosphoester transfer reactions with a high degree of specificity, often cleaving only one of several phosphoesters in an oligonucleotide substrate (Cech et al., Cell. 1981 Dec;27(3 Pt 2):487-96; Michel and Westhof, J Mol Biol. 1990 Dec 5;216(3):585-610; Reinhold-Hurek and Shub, Nature. 1992 May 14;357(6374):173-6). This specificity has been attributed to the requirement that the substrate bind via specific base-pairing interactions to the internal guide sequence (“IGS”) of the ribozyme prior to chemical reaction.

Six basic varieties of naturally-occurring enzymatic RNAs are known presently. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nature of a ribozyme is advantageous over many technologies, such as antisense technology (where a nucleic acid molecule simply binds to a nucleic acid target to block its translation) since the concentration of ribozyme necessary to affect a therapeutic treatment is lower than that of an antisense oligonucleotide. This advantage reflects the ability of the ribozyme to act enzymatically. Thus, a single ribozyme molecule is able to cleave many molecules of target RNA. In addition, the ribozyme is a highly specific inhibitor, with the specificity of inhibition depending not only on the base pairing mechanism of binding to the target RNA, but also on the mechanism of target RNA cleavage. Single mismatches, or base-substitutions, near the site of cleavage can completely eliminate catalytic activity of a ribozyme. Similar mismatches in antisense molecules do not prevent their action (Woolf et al., Proc Natl Acad Sci U S A. 1992 Aug 15;89(16):7305-9). Thus, the specificity of action of a ribozyme is greater than that of an antisense oligonucleotide binding the same RNA site.

The enzymatic nucleic acid molecule may be formed in a hammerhead, hairpin, a hepatitis δ virus, group I intron or RNaseP RNA (in association with an RNA guide sequence) or Neurospora VS RNA motif. Examples of hammerhead motifs are described by Rossi et al. Nucleic Acids Res. 1992 Sep 11;20(17):4559-65. Examples of hairpin motifs are described by Hampel et al. (Eur. Pat. Appl. Publ. No. EP 0360257), Hampel and Tritz, Biochemistry 1989 Jun 13;28(12):4929-33; Hampel et al., Nucleic Acids Res. 1990 Jan 25;18(2):299-304 and U.S. Pat. No. 5,631,359. An example of the hepatitis δ virus motif is described by Perrotta and Been, Biochemistry. 1992 Dec 1;31(47):11843-52; an example of the RNaseP motif is described by Guerrier-Takada et al., Cell. 1983 Dec;35(3 Pt 2):849-57; Neurospora VS RNA ribozyme motif is described by Collins (Saville and Collins, Cell. 1990 May 18;61(4):685-96; Saville and Collins, Proc Natl Acad Sci U S A. 1991 Oct 1;88(19):8826-30; Collins and Olive, Biochemistry. 1993 Mar 23;32(11):2795-9); and an example of the Group I intron is described in (U.S. Pat. No. 4,987,071). All that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule. Thus the ribozyme constructs need not be limited to specific motifs mentioned herein.

Ribozymes may be designed as described in Int. Pat. Appl. Publ. No. WO 93/23569 and Int. Pat. Appl. Publ. No. WO 94/02595, each specifically incorporated herein by reference) and synthesized to be tested in vitro and in vivo, as described. Such ribozymes can also be optimized for delivery. While specific examples are provided, those in the art will recognize that equivalent RNA targets in other species can be utilized when necessary.

Ribozyme activity can be optimized by altering the length of the ribozyme binding arms, or chemically synthesizing ribozymes with modifications that prevent their degradation by serum ribonucleases (see e.g., Int. Pat. Appl. Publ. No. WO 92/07065; Int. Pat. Appl. Publ. No. WO 93/15187; Int. Pat. Appl. Publ. No. WO 91/03162; Eur. Pat. Appl. Publ. No. 92110298.4; U.S. Pat. No. 5,334,711; and Int. Pat. Appl. Publ. No. WO 94/13688, which describe various chemical modifications that can be made to the sugar moieties of enzymatic RNA molecules), modifications which enhance their efficacy in cells, and removal of stem II bases to shorten RNA synthesis times and reduce chemical requirements.

Sullivan et al. (Int. Pat. Appl. Publ. No. WO 94/02595) describes the general methods for delivery of enzymatic RNA molecules. Ribozymes may be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, ribozymes may be directly delivered ex vivo to cells or tissues with or without the aforementioned vehicles. Alternatively, the RNA/vehicle combination may be locally delivered by direct inhalation, by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. More detailed descriptions of ribozyme delivery and administration are provided in Int. Pat. Appl. Publ. No. WO 94/02595 and Int. Pat. Appl. Publ. No. WO 93/23569, each specifically incorporated herein by reference.

Another means of accumulating high concentrations of a ribozyme(s) within cells is to incorporate the ribozyme-encoding sequences into a DNA expression vector. Transcription of the ribozyme sequences are driven from a promoter for eukaryotic RNA polymerase I (pol I), RNA polymerase II (pol II), or RNA polymerase III (pol III). Transcripts from pol II or pol III promoters will be expressed at high levels in all cells; the levels of a given pol II promoter in a given cell type will depend on the nature of the gene regulatory sequences (enhancers, silencers, etc.) present nearby. Prokaryotic RNA polymerase promoters may also be used, providing that the prokaryotic RNA polymerase enzyme is expressed in the appropriate cells Ribozymes expressed from such promoters have been shown to function in mammalian cells. Such transcription units can be incorporated into a variety of vectors for introduction into mammalian cells, including but not restricted to, plasmid DNA vectors, viral DNA vectors (such as adenovirus or adeno-associated vectors), or viral RNA vectors (such as retroviral, semliki forest virus, sindbis virus vectors).

In another embodiment of the invention, peptide nucleic acids (PNAs) compositions are provided. PNA is a DNA mimic in which the nucleobases are attached to a pseudopeptide backbone (Good and Nielsen, Antisense Nucleic Acid Drug Dev. 1997 7(4) 431-37). PNA is able to be utilized in a number methods that traditionally have used RNA or DNA. Often PNA sequences perform better in techniques than the corresponding RNA or DNA sequences and have utilities that are not inherent to RNA or DNA. A review of PNA including methods of making, characteristics of, and methods of using, is provided by Corey (Trends Biotechnol 1997 Jun;15(6):224-9). As such, in certain embodiments, one may prepare PNA sequences that are complementary to one or more portions of the ACE mRNA sequence, and such PNA compositions may be used to regulate, alter, decrease, or reduce the translation of ACE-specific mRNA, and thereby alter the level of ACE activity in a host cell to which such PNA compositions have been administered.

PNAs have 2-aminoethyl-glycine linkages replacing the normal phosphodiester backbone of DNA (Nielsen et al., Science 1991 Dec 6;254(5037):1497-500; Hanvey et al., Science. 1992 Nov 27;258(5087):1481-5; Hyrup and Nielsen, Bioorg Med Chem. 1996 Jan;4(l):5-23). This chemistry has three important consequences: firstly, in contrast to DNA or phosphorothioate oligonucleotides, PNAs are neutral molecules; secondly, PNAs are achiral, which avoids the need to develop a stereoselective synthesis; and thirdly, PNA synthesis uses standard Boc or Fmoc protocols for solid-phase peptide synthesis, although other methods, including a modified Merrifield method, have been used.

PNA monomers or ready-made oligomers are commercially available from PerSeptive Biosystems (Framingham, Mass.). PNA syntheses by either Boc or Fmoc protocols are straightforward using manual or automated protocols (Norton et al., Bioorg Med Chem. 1995 Apr;3(4):437-45). The manual protocol lends itself to the production of chemically modified PNAs or the simultaneous synthesis of families of closely related PNAs.

As with peptide synthesis, the success of a particular PNA synthesis will depend on the properties of the chosen sequence. For example, while in theory PNAs can incorporate any combination of nucleotide bases, the presence of adjacent purines can lead to deletions of one or more residues in the product. In expectation of this difficulty, it is suggested that, in producing PNAs with adjacent purines, one should repeat the coupling of residues likely to be added inefficiently. This should be followed by the purification of PNAs by reverse-phase high-pressure liquid chromatography, providing yields and purity of product similar to those observed during the synthesis of peptides.

Modifications of PNAs for a given application may be accomplished by coupling amino acids during solid-phase synthesis or by attaching compounds that contain a carboxylic acid group to the exposed N-terminal amine. Alternatively, PNAs can be modified after synthesis by coupling to an introduced lysine or cysteine. The ease with which PNAs can be modified facilitates optimization for better solubility or for specific functional requirements. Once synthesized, the identity of PNAs and their derivatives can be confirmed by mass spectrometry. Several studies have made and utilized modifications of PNAs (for example, Norton et al., Bioorg Med Chem. 1995 Apr;3(4):437-45; Petersen et al., J Pept Sci. 1995 May-Jun;1(3):175-83; Orum et al., Biotechniques. 1995 Sep;19(3):472-80; Footer et al., Biochemistry. 1996 Aug 20;35(33):10673-9; Griffith et al., Nucleic Acids Res. 1995 Aug 1 1;23(15):3003-8; Pardridge et al., Proc Natl Acad Sci U S A. 1995 Jun 6;92(12):5592-6; Boffa et al., Proc Natl Acad Sci U S A. 1995 Mar 14;92(6):1901-5; Gambacorti-Passerini et al., Blood. 1996 Aug 15;88(4):1411-7; Armitage et al., Proc Natl Acad Sci U S A. 1997 Nov 11;94(23):12320-5; Seeger et al., Biotechniques. 1997 Sep;23(3):512-7). U.S. Pat. No. 5,700,922 discusses PNA-DNA-PNA chimeric molecules and their uses in diagnostics, modulating protein in organisms, and treatment of conditions susceptible to therapeutics.

Methods of characterizing the antisense binding properties of PNAs are discussed in Rose (Anal Chem. 1993 Dec 15;65(24):3545-9) and Jensen et al. (Biochemistry. 1997 Apr 22;36(16):5072-7). Rose uses capillary gel electrophoresis to determine binding of PNAs to their complementary oligonucleotide, measuring the relative binding kinetics and stoichiometry. Similar types of measurements were made by Jensen et al. using BIAcore™ technology.

Other applications of PNAs that have been described and will be apparent to the skilled artisan include use in DNA strand invasion, antisense inhibition, mutational analysis, enhancers of transcription, nucleic acid purification, isolation of transcriptionally active genes, blocking of transcription factor binding, genome cleavage, biosensors, in situ hybridization, and the like.

Polynucleotide Identification, Characterization and Expression

Polynucleotides compositions of the present invention may be identified, prepared and/or manipulated using any of a variety of well established techniques (see generally, Sambrook et al., Molecular Cloning: A Laboratory Manual, Cold Spring Harbor Laboratories, Cold Spring Harbor, N.Y., 1989, and other like references). For example, a polynucleotide may be identified, as described in more detail below, by screening a microarray of cDNAs for tumor-associated expression (i.e., expression that is at least two fold greater in a tumor than in normal tissue, as determined using a representative assay provided herein). Such screens may be performed, for example, using the microarray technology of Affymetrix, Inc. (Santa Clara, Calif.) according to the manufacturer's instructions (and essentially as described by Schena et al., Proc. Natl. Acad. Sci. USA 93:10614-10619, 1996 and Heller et al., Proc. Natl. Acad. Sci. USA 94:2150-2155, 1997). Alternatively, polynucleotides may be amplified from cDNA prepared from cells expressing the proteins described herein, such as tumor cells.

Many template dependent processes are available to amplify a target sequences of interest present in a sample. One of the best known amplification methods is the polymerase chain reaction (PCR™) which is described in detail in U.S. Pat. Nos. 4,683,195, 4,683,202 and 4,800,159, each of which is incorporated herein by reference in its entirety. Briefly, in PCR™, two primer sequences are prepared which are complementary to regions on opposite complementary strands of the target sequence. An excess of deoxynucleoside triphosphates is added to a reaction mixture along with a DNA polymerase (e.g., Taq polymerase). If the target sequence is present in a sample, the primers will bind to the target and the polymerase will cause the primers to be extended along the target sequence by adding on nucleotides. By raising and lowering the temperature of the reaction mixture, the extended primers will dissociate from the target to form reaction products, excess primers will bind to the target and to the reaction product and the process is repeated. Preferably reverse transcription and PCR™ amplification procedure may be performed in order to quantify the amount of mRNA amplified. Polymerase chain reaction methodologies are well known in the art.

Any of a number of other template dependent processes, many of which are variations of the PCR™ amplification technique, are readily known and available in the art. Illustratively, some such methods include the ligase chain reaction (referred to as LCR), described, for example, in Eur. Pat. Appl. Publ. No. 320,308 and U.S. Pat. No. 4,883,750; Qbeta Replicase, described in PCT Intl. Pat. Appl. Publ. No. PCT/US87/00880; Strand Displacement Amplification (SDA) and Repair Chain Reaction (RCR). Still other amplification methods are described in Great Britain Pat. Appl. No. 2 202 328, and in PCT Intl. Pat. Appl. Publ. No. PCT/US89/01025. Other nucleic acid amplification procedures include transcription-based amplification systems (TAS) (PCT Intl. Pat. Appl. Publ. No. WO 88/10315), including nucleic acid sequence based amplification (NASBA) and 3SR. Eur. Pat. Appl. Publ. No. 329,822 describes a nucleic acid amplification process involving cyclically synthesizing single-stranded RNA (“ssRNA”), ssDNA, and double-stranded DNA (dsDNA). PCT Intl. Pat. Appl. Publ. No. WO 89/06700 describes a nucleic acid sequence amplification scheme based on the hybridization of a promoter/primer sequence to a target single-stranded DNA (“ssDNA”) followed by transcription of many RNA copies of the sequence. Other amplification methods such as “RACE” (Frohman, 1990), and “one-sided PCR” (Ohara, 1989) are also well-known to those of skill in the art.

An amplified portion of a polynucleotide of the present invention may be used to isolate a full length gene from a suitable library (e.g., a tumor cDNA library) using well known techniques. Within such techniques, a library (cDNA or genomic) is screened using one or more polynucleotide probes or primers suitable for amplification. Preferably, a library is size-selected to include larger molecules. Random primed libraries may also be preferred for identifying 5′ and upstream regions of genes. Genomic libraries are preferred for obtaining introns and extending 5′ sequences.

For hybridization techniques, a partial sequence may be labeled (e.g., by nick-translation or end-labeling with ³²p) using well known techniques. A bacterial or bacteriophage library is then generally screened by hybridizing filters containing denatured bacterial colonies (or lawns containing phage plaques) with the labeled probe (see Sambrook et al., Molecular Cloning: A Laboratory Manual, Cold Spring Harbor Laboratories, Cold Spring Harbor, N.Y., 1989). Hybridizing colonies or plaques are selected and expanded, and the DNA is isolated for further analysis. cDNA clones may be analyzed to determine the amount of additional sequence by, for example, PCR using a primer from the partial sequence and a primer from the vector. Restriction maps and partial sequences may be generated to identify one or more overlapping clones. The complete sequence may then be determined using standard techniques, which may involve generating a series of deletion clones. The resulting overlapping sequences can then assembled into a single contiguous sequence. A full length cDNA molecule can be generated by ligating suitable fragments, using well known techniques.

Alternatively, amplification techniques, such as those described above, can be useful for obtaining a fall length coding sequence from a partial cDNA sequence. One such amplification technique is inverse PCR (see Triglia et al., Nucl. Acids Res. 16:8186, 1988), which uses restriction enzymes to generate a fragment in the known region of the gene. The fragment is then circularized by intramolecular ligation and used as a template for PCR with divergent primers derived from the known region. Within an alternative approach, sequences adjacent to a partial sequence may be retrieved by amplification with a primer to a linker sequence and a primer specific to a known region. The amplified sequences are typically subjected to a second round of amplification with the same linker primer and a second primer specific to the known region. A variation on this procedure, which employs two primers that initiate extension in opposite directions from the known sequence, is described in WO 96/38591. Another such technique is known as “rapid amplification of cDNA ends” or RACE. This technique involves the use of an internal primer and an external primer, which hybridizes to a polyA region or vector sequence, to identify sequences that are 5′ and 3′ of a known sequence. Additional techniques include capture PCR (Lagerstrom et al., PCR Methods Applic. 1:111-19, 1991) and walking PCR (Parker et al., Nucl. Acids. Res. 19:3055-60, 1991). Other methods employing amplification may also be employed to obtain a full length cDNA sequence.

In certain instances, it is possible to obtain a full length cDNA sequence by analysis of sequences provided in an expressed sequence tag (EST) database, such as that available from GenBank. Searches for overlapping ESTs may generally be performed using well known programs (e.g., NCBI BLAST searches), and such ESTs may be used to generate a contiguous full length sequence. Full length DNA sequences may also be obtained by analysis of genomic fragments.

In other embodiments of the invention, polynucleotide sequences or fragments thereof which encode polypeptides of the invention, or fusion proteins or functional equivalents thereof, may be used in recombinant DNA molecules to direct expression of a polypeptide in appropriate host cells. Due to the inherent degeneracy of the genetic code, other DNA sequences that encode substantially the same or a functionally equivalent amino acid sequence may be produced and these sequences may be used to clone and express a given polypeptide.

As will be understood by those of skill in the art, it may be advantageous in some instances to produce polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence.

Moreover, the polynucleotide sequences of the present invention can be engineered using methods generally known in the art in order to alter polypeptide encoding sequences for a variety of reasons, including but not limited to, alterations which modify the cloning, processing, and/or expression of the gene product. For example, DNA shuffling by random fragmentation and PCR reassembly of gene fragments and synthetic oligonucleotides may be used to engineer the nucleotide sequences. In addition, site-directed mutagenesis may be used to insert new restriction sites, alter glycosylation patterns, change codon preference, produce splice variants, or introduce mutations, and so forth.

In another embodiment of the invention, natural, modified, or recombinant nucleic acid sequences may be ligated to a heterologous sequence to encode a fusion protein. For example, to screen peptide libraries for inhibitors of polypeptide activity, it may be useful to encode a chimeric protein that can be recognized by a commercially available antibody. A fusion protein may also be engineered to contain a cleavage site located between the polypeptide-encoding sequence and the heterologous protein sequence, so that the polypeptide may be cleaved and purified away from the heterologous moiety.

Sequences encoding a desired polypeptide may be synthesized, in whole or in part, using chemical methods well known in the art (see Caruthers, M. H. et al. (1980) Nucl. Acids Res. Symp. Ser. 215-223, Horn, T. et al. (1980) Nucl. Acids Res. Symp. Ser. 225-232). Alternatively, the protein itself may be produced using chemical methods to synthesize the amino acid sequence of a polypeptide, or a portion thereof. For example, peptide synthesis can be performed using various solid-phase techniques (Roberge, J. Y. et al. (1995) Science 269:202-204) and automated synthesis may be achieved, for example, using the ABI 431 A Peptide Synthesizer (Perkin Elmer, Palo Alto, Calif.).

A newly synthesized peptide may be substantially purified by preparative high performance liquid chromatography (e.g., Creighton, T. (1983) Proteins, Structures and Molecular Principles, W H Freeman and Co., New York, N.Y.) or other comparable techniques available in the art. The composition of the synthetic peptides may be confirmed by amino acid analysis or sequencing (e.g., the Edman degradation procedure). Additionally, the amino acid sequence of a polypeptide, or any part thereof, may be altered during direct synthesis and/or combined using chemical methods with sequences from other proteins, or any part thereof, to produce a variant polypeptide.

In order to express a desired polypeptide, the nucleotide sequences encoding the polypeptide, or functional equivalents, may be inserted into appropriate expression vector, i.e., a vector which contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described, for example, in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York. N.Y.

A variety of expression vector/host systems may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

The “control elements” or “regulatory sequences” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the PBLUESCRIPT phagemid (Stratagene, La Jolla, Calif.) or PSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, any of a number of expression vectors may be selected depending upon the use intended for the expressed polypeptide. For example, when large quantities are needed, for example for the induction of antibodies, vectors which direct high level expression of fusion proteins that are readily purified may be used. Such vectors include, but are not limited to, the multifunctional E. Coli cloning and expression vectors such as BLUESCRIPT (Stratagene), in which the sequence encoding the polypeptide of interest may be ligated into the vector in frame with sequences for the amino-terminal Met and the subsequent 7 residues of beta.-galactosidase so that a hybrid protein is produced; pIN vectors (Van Heeke, G. and S. M. Schuster (1989) J. Biol. Chem. 264:5503-5509); and the like. pGEX Vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH may be used. For reviews, see Ausubel et al. (supra) and Grant et al. (1987) Methods Enzymol. 153:516-544.

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, N. (1987) EMBO J. 6:307-31 1. Alternatively, plant promoters such as the small subunit of RUBISCO or heat shock promoters may be used (Coruzzi, G. et al. (1984) EMBO J. 3:1671-1680; Broglie, R. et al. (1984) Science 224:838-843; and Winter, J. et al. (1991) Results Probl. Cell Differ. 17:85-105). These constructs can be introduced into plant cells by direct DNA transformation or pathogen-mediated transfection. Such techniques are described in a number of generally available reviews (see, for example, Hobbs, S. or Murry, L. E. in McGraw Hill Yearbook of Science and Technology (1992) McGraw Hill, New York, N.Y.; pp. 191-196).

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia larvae. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia larvae in which the polypeptide of interest may be expressed (Engelhard, E. K. et al. (1994) Proc. Natl. Acad. Sci. 91 :3224-3227).

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan, J. and Shenk, T. (1984) Proc. Natl. Acad. Sci. 81:3655-3659). In addition, transcription enhancers, such as the Rous sarcoma virus (RSV) enhancer, may be used to increase expression in mammalian host cells.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf, D. et al. (1994) Results Probl. Cell Differ. 20:125-162).

In addition, a host cell strain may be chosen for its ability to modulate the expression of the inserted sequences or to process the expressed protein in the desired fashion. Such modifications of the polypeptide include, but are not limited to, acetylation, carboxylation. glycosylation, phosphorylation, lipidation, and acylation. Post-translational processing which cleaves a “prepro” form of the protein may also be used to facilitate correct insertion, folding and/or function. Different host cells such as CHO, COS, HeLa, MDCK, HEK293, and W138, which have specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines which stably express a polynucleotide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase (Wigler, M. et al. (1977) Cell 11:223-32) and adenine phosphoribosyltransferase (Lowy, I. et al. (1990) Cell 22:817-23) genes which can be employed in tk.sup.- or aprt.sup.-cells, respectively. Also, antimetabolite, antibiotic or herbicide resistance can be used as the basis for selection; for example, dhfr which confers resistance to methotrexate (Wigler, M. et al. (1980) Proc. Natl. Acad. Sci. 77:3567-70); npt, which confers resistance to the aminoglycosides, neomycin and G-418 (Colbere-Garapin, F. et al (1981) J. Mol. Biol. 150:1-14); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman, S. C. and R. C. Mulligan (1988) Proc. Natl. Acad. Sci. 85:8047-51). The use of visible markers has gained popularity with such markers as anthocyanins, beta-glucuronidase and its substrate GUS, and luciferase and its substrate luciferin, being widely used not only to identify transformants, but also to quantify the amount of transient or stable protein expression attributable to a specific vector system (Rhodes, C. A. et al. (1995) Methods Mol. Biol. 55:121-131).

Although the presence/absence of marker gene expression suggests that the gene of interest is also present, its presence and expression may need to be confirmed. For example, if the sequence encoding a polypeptide is inserted within a marker gene sequence, recombinant cells containing sequences can be identified by the absence of marker gene function. Alternatively, a marker gene can be placed in tandem with a polypeptide-encoding sequence under the control of a single promoter. Expression of the marker gene in response to induction or selection usually indicates expression of the tandem gene as well.

Alternatively, host cells that contain and express a desired polynucleotide sequence may be identified by a variety of procedures known to those of skill in the art. These procedures include, but are not limited to, DNA-DNA or DNA-RNA hybridizations and protein bioassay or immunoassay techniques which include, for example, membrane, solution, or chip based technologies for the detection and/or quantification of nucleic acid or protein.

A variety of protocols for detecting and measuring the expression of polynucleotide-encoded products, using either polyclonal or monoclonal antibodies specific for the product are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on a given polypeptide may be preferred for some applications, but a competitive binding assay may also be employed. These and other assays are described, among other places, in Hampton, R. et al. (1990; Serological Methods, a Laboratory Manual, APS Press, St Paul. Minn.) and Maddox, D. E. et al. (1983; J. Exp. Med. 158:1211-1216).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with a polynucleotide sequence of interest may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides of the invention may be designed to contain signal sequences which direct secretion of the encoded polypeptide through a prokaryotic or eukaryotic cell membrane. Other recombinant constructions may be used to join sequences encoding a polypeptide of interest to nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences such as those specific for Factor XA or enterokinase (Invitrogen. San Diego, Calif.) between the purification domain and the encoded polypeptide may be used to facilitate purification. One such expression vector provides for expression of a fusion protein containing a polypeptide of interest and a nucleic acid encoding 6 histidine residues preceding a thioredoxin or an enterokinase cleavage site. The histidine residues facilitate purification on IMIAC (immobilized metal ion affinity chromatography) as described in Porath, J. et al. (1992, Prot. Exp. Purif 3:263-281) while the enterokinase cleavage site provides a means for purifying the desired polypeptide from the fusion protein. A discussion of vectors which contain fusion proteins is provided in Kroll, D. J. et al. (1993; DNA Cell Biol. 12:441-453).

In addition to recombinant production methods, polypeptides of the invention, and fragments thereof, may be produced by direct peptide synthesis using solid-phase techniques (Merrifield J. (1963) J. Am. Chem. Soc. 85:2149-2154). Protein synthesis may be performed using manual techniques or by automation. Automated synthesis may be achieved, for example, using Applied Biosystems 431A Peptide Synthesizer (Perkin Elmer). Alternatively, various fragments may be chemically synthesized separately and combined using chemical methods to produce the full length molecule.

Antibody Compositions, Fragrments Thereof and Other Binding Agents

According to another aspect, the present invention further provides binding agents, such as antibodies and antigen-binding fragments thereof, that exhibit immunological binding to a tumor polypeptide disclosed herein, or to a portion, variant or derivative thereof. An antibody, or antigen-binding fragment thereof, is said to “specifically bind,” “immunogically bind,” and/or is “immunologically reactive” to a polypeptide of the invention if it reacts at a detectable level (within, for example, an ELISA assay) with the polypeptide, and does not react detectably with unrelated polypeptides under similar conditions.

Immunological binding, as used in this context, generally refers to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kₒₙ) and the “off rate constant” (K_{off}) can be determined by calculation of the concentrations and the actual rates of association and dissociation. The ratio of K_{off}/Kₒₙ enables cancellation of all parameters not related to affinity, and is thus equal to the dissociation constant K_{d}. See, generally, Davies et al. (1990) Annual Rev. Biochem. 59:439-473.

An “antigen-binding site,” or “binding portion” of an antibody refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FRs”. Thus the term “FR” refers to amino acid sequences which are naturally found between and adjacent to hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

Binding agents may be further capable of differentiating between patients with and without a cancer, such as colon cancer, using the representative assays provided herein. For example, antibodies or other binding agents that bind to a tumor protein will preferably generate a signal indicating the presence of a cancer in at least about 20% of patients with the disease, more preferably at least about 30% of patients. Alternatively, or in addition, the antibody will generate a negative signal indicating the absence of the disease in at least about 90% of individuals without the cancer. To determine whether a binding agent satisfies this requirement, biological samples (e.g., blood, sera, sputum, urine and/or tumor biopsies) from patients with and without a cancer (as determined using standard clinical tests) may be assayed as described herein for the presence of polypeptides that bind to the binding agent. Preferably, a statistically significant number of samples with and without the disease will be assayed. Each binding agent should satisfy the above criteria; however, those of ordinary skill in the art will recognize that binding agents may be used in combination to improve sensitivity.

Any agent that satisfies the above requirements may be a binding agent. For example, a binding agent may be a ribosome, with or without a peptide component, an RNA molecule or a polypeptide. In a preferred embodiment, a binding agent is an antibody or an antigen-binding fragment thereof. Antibodies may be prepared by any of a variety of techniques known to those of ordinary skill in the art. See, e.g., Harlow and Lane, Antibodies: A Laboratory Manual, Cold Spring Harbor Laboratory, 1988. In general, antibodies can be produced by cell culture techniques, including the generation of monoclonal antibodies as described herein, or via transfection of antibody genes into suitable bacterial or mammalian cell hosts, in order to allow for the production of recombinant antibodies. In one technique, an immunogen comprising the polypeptide is initially injected into any of a wide variety of mammals (e.g., mice, rats, rabbits, sheep or goats). In this step, the polypeptides of this invention may serve as the immunogen without modification. Alternatively, particularly for relatively short polypeptides, a superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Monoclonal antibodies specific for an antigenic polypeptide of interest may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the polypeptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the polypeptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

A number of therapeutically useful molecules are known in the art which comprise antigen-binding sites that are capable of exhibiting immunological binding properties of an antibody molecule. The proteolytic enzyme papain preferentially cleaves IgG molecules to yield several fragments, two of which (the “F(ab)” fragments) each comprise a covalent heterodimer that includes an intact antigen-binding site. The enzyme pepsin is able to cleave IgG molecules to provide several fragments, including the “F(ab′)₂” fragment which comprises both antigen-binding sites. An “Fv” fragment can be produced by preferential proteolytic cleavage of an IgM, and on rare occasions IgG or IgA immunoglobulin molecule. Fv fragments are, however, more commonly derived using recombinant techniques known in the art. The Fv fragment includes a non-covalent V_{H}::V_{L} heterodimer including an antigen-binding site which retains much of the antigen recognition and binding capabilities of the native antibody molecule. Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

A single chain Fv (“sFv”) polypeptide is a covalently linked V_{H}::V_{L} heterodimer which is expressed from a gene fusion including V_{H}- and V_{L}-encoding genes linked by a peptide-encoding linker. Huston et al. (1988) Proc. Nat. Acad. Sci. USA 85(16):5879-5883. A number of methods have been described to discern chemical structures for converting the naturally aggregated—but chemically separated—light and heavy polypeptide chains from an antibody V region into an sFv molecule which will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

Each of the above-described molecules includes a heavy chain and a light chain CDR set, respectively interposed between a heavy chain and a light chain FR set which provide support to the CDRS and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRS. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures—regardless of the precise CDR amino acid sequence. Further, certain FR residues are known to participate in non-covalent interdomain contacts which stabilize the interaction of the antibody heavy and light chains.

A number of “humanized” antibody molecules comprising an antigen-binding site derived from a non-human immunoglobulin have been described, including chimeric antibodies having rodent V regions and their associated CDRs fused to human constant domains (Winter et al. (1991) Nature 349:293-299; Lobuglio et al. (1989) Proc. Nat. Acad. Sci. USA 86:4220-4224; Shaw et al. (1987) J Immunol. 138:4534-4538; and Brown et al. (1987) Cancer Res. 47:3577-3583), rodent CDRs grafted into a human supporting FR prior to fusion with an appropriate human antibody constant domain (Riechmann et al. (1988) Nature 332:323-327; Verhoeyen et al. (1988) Science 239:1534-1536; and Jones et al. (1986) Nature 321:522-525), and rodent CDRs supported by recombinantly veneered rodent FRs (European Patent Publication No. 519,596, published Dec. 23, 1992). These “humanized” molecules are designed to minimize unwanted immunological response toward rodent antihuman antibody molecules which limits the duration and effectiveness of therapeutic applications of those moieties in human recipients.

As used herein, the terms “veneered FRs” and “recombinantly veneered FRs” refer to the selective replacement of FR residues from, e.g., a rodent heavy or light chain V region, with human FR residues in order to provide a xenogeneic molecule comprising an antigen-binding site which retains substantially all of the native FR polypeptide folding structure. Veneering techniques are based on the understanding that the ligand binding characteristics of an antigen-binding site are determined primarily by the structure and relative disposition of the heavy and light chain CDR sets within the antigen-binding surface. Davies et al. (1990) Ann. Rev. Biochem. 59:439-473. Thus, antigen binding specificity can be preserved in a humanized antibody only wherein the CDR structures, their interaction with each other, and their interaction with the rest of the V region domains are carefully maintained. By using veneering techniques, exterior (e.g., solvent-accessible) FR residues which are readily encountered by the immune system are selectively replaced with human residues to provide a hybrid molecule that comprises *: i either a weakly immunogenic, or substantially non-immunogenic veneered surface.

The process of veneering makes use of the available sequence data for human antibody variable domains compiled by Kabat et al., in Sequences of Proteins of Immunological Interest, 4th ed., (U.S. Dept. of Health and Human Services, U.S. Government Printing Office, 1987), updates to the Kabat database, and other accessible U.S. and foreign databases (both nucleic acid and protein). Solvent accessibilities of V region amino acids can be deduced from the known three-dimensional structure for human and murine antibody fragments. There are two general steps in veneering a murine antigen-binding site. Initially, the FRs of the variable domains of an antibody molecule of interest are compared with corresponding FR sequences of human variable domains obtained from the above-identified sources. The most homologous human V regions are then compared residue by residue to corresponding murine amino acids. The residues in the murine FR which differ from the human counterpart are replaced by the residues present in the human moiety using recombinant techniques well known in the art. Residue switching is only carried out with moieties which are at least partially exposed (solvent accessible), and care is exercised in the replacement of amino acid residues which may have a significant effect on the tertiary structure of V region domains, such as proline, glycine and charged amino acids.

In this manner, the resultant “veneered” murine antigen-binding sites are thus designed to retain the murine CDR residues, the residues substantially adjacent to the CDRs, the residues identified as buried or mostly buried (solvent inaccessible), the residues believed to participate in non-covalent (e.g., electrostatic and hydrophobic) contacts between heavy and light chain domains, and the residues from conserved structural regions of the FRs which are believed to influence the “canonical” tertiary structures of the CDR loops. These design criteria are then used to prepare recombinant nucleotide sequences which combine the CDRs of both the heavy and light chain of a murine antigen-binding site into human-appearing FRs that can be used to transfect mammalian cells for the expression of recombinant human antibodies which exhibit the antigen specificity of the murine antibody molecule.

In another embodiment of the invention, monoclonal antibodies of the present invention may be coupled to one or more therapeutic agents. Suitable agents in this regard include radionuclides, differentiation inducers, drugs, toxins, and derivatives thereof. Preferred radionuclides include ⁹⁰Y, ¹²³I, ¹²⁵I, ¹³¹I, ¹⁸⁶Re, ¹⁸⁸Re, ²¹¹At, and ²¹²Bi. Preferred drugs include methotrexate, and pyrimidine and purine analogs. Preferred differentiation inducers include phorbol esters and butyric acid. Preferred toxins include ricin, abrin, diptheria toxin, cholera toxin, gelonin, Pseudomonas exotoxin, Shigella toxin, and pokeweed antiviral protein.

A therapeutic agent may be coupled (e.g., covalently bonded) to a suitable monoclonal antibody either directly or indirectly (e.g., via a linker group). A direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

Alternatively, it may be desirable to couple a therapeutic agent and an antibody via a linker group. A linker group can function as a spacer to distance an antibody from an agent in order to avoid interference with binding capabilities. A linker group can also serve to increase the chemical reactivity of a substituent on an agent or an antibody, and thus increase the coupling efficiency. An increase in chemical reactivity may also facilitate the use of agents, or functional groups on agents, which otherwise would not be possible.

It will be evident to those skilled in the art that a variety of bifunctional or polyfunctional reagents, both homo- and hetero-functional (such as those described in the catalog of the Pierce Chemical Co., Rockford, Ill.), may be employed as the linker group. Coupling may be effected, for example, through amino groups, carboxyl groups, sulfhydryl groups or oxidized carbohydrate residues. There are numerous references describing such methodology, e.g., U.S. Pat. No. 4,671,958, to Rodwell et al.

Where a therapeutic agent is more potent when free from the antibody portion of the immunoconjugates of the present invention, it may be desirable to use a linker group which is cleavable during or upon internalization into a cell. A number of different cleavable linker groups have been described. The mechanisms for the intracellular release of an agent from these linker groups include cleavage by reduction of a disulfide bond (e.g., U.S. Pat. No. 4,489,710, to Spitler), by irradiation of a photolabile bond (e.g., U.S. Pat. No. 4,625,014, to Senter et al.), by hydrolysis of derivatized amino acid side chains (e.g., U.S. Pat. No. 4,638,045, to Kohn et al.), by serum complement-mediated hydrolysis (e.g., U.S. Pat. No. 4,671,958, to Rodwell et al.), and acid-catalyzed hydrolysis (e.g., U.S. Pat. No. 4,569,789, to Blattler et al.).

It may be desirable to couple more than one agent to an antibody. In one embodiment, multiple molecules of an agent are coupled to one antibody molecule. In another embodiment, more than one type of agent may be coupled to one antibody. Regardless of the particular embodiment, immunoconjugates with more than one agent may be prepared in a variety of ways. For example, more than one agent may be coupled directly to an antibody molecule, or linkers that provide multiple sites for attachment can be used. Alternatively, a carrier can be used.

A carrier may bear the agents in a variety of ways, including covalent bonding either directly or via a linker group. Suitable carriers include proteins such as albumins (e.g., U.S. Pat. No. 4,507,234, to Kato et al.), peptides and polysaccharides such as aminodextran (e.g., U.S. Pat. No. 4,699,784, to Shih et al.). A carrier may also bear an agent by noncovalent bonding or by encapsulation, such as within a liposome vesicle (e.g., U.S. Pat. Nos. 4,429,008 and 4,873,088). Carriers specific for radionuclide agents include radiohalogenated small molecules and chelating compounds. For example, U.S. Pat. No. 4,735,792 discloses representative radiohalogenated small molecules and their synthesis. A radionuclide chelate may be formed from chelating compounds that include those containing nitrogen and sulfur atoms as the donor atoms for binding the metal, or metal oxide, radionuclide. For example, U.S. Pat. No. 4,673,562, to Davison et al. discloses representative chelating compounds and their synthesis.

T Cell Compositions

The present invention, in another aspect, provides T cells specific for a tumor polypeptide disclosed herein, or for a variant or derivative thereof. Such cells may generally be prepared in vitro or ex vivo, using standard procedures. For example, T cells may be isolated from bone marrow, peripheral blood, or a fraction of bone marrow or peripheral blood of a patient, using a commercially available cell separation system, such as the Isolex™ System, available from Nexell Therapeutics, Inc. (Irvine, Calif.; see also U.S. Pat. No. 5,240,856; U.S. Pat. No. 5,215,926; WO 89/06280; WO 91/16116 and WO 92/07243). Alternatively, T cells may be derived from related or unrelated humans, non-human mammals, cell lines or cultures.

T cells may be stimulated with a polypeptide, polynucleotide encoding a polypeptide and/or an antigen presenting cell (APC) that expresses such a polypeptide. Such stimulation is performed under conditions and for a time sufficient to permit the generation of T cells that are specific for the polypeptide of interest. Preferably, a tumor polypeptide or polynucleotide of the invention is present within a delivery vehicle, such as a microsphere, to facilitate the generation of specific T cells.

T cells are considered to be specific for a polypeptide of the present invention if the T cells specifically proliferate, secrete cytokines or kill target cells coated with the polypeptide or expressing a gene encoding the polypeptide. T cell specificity may be evaluated using any of a variety of standard techniques. For example, within a chromium release assay or proliferation assay, a stimulation index of more than two fold increase in lysis and/or proliferation, compared to negative controls, indicates T cell specificity. Such assays may be performed, for example, as described in Chen et al., Cancer Res. 54:1065-1070, 1994. Alternatively, detection of the proliferation of T cells may be accomplished by a variety of known techniques. For example, T cell proliferation can be detected by measuring an increased rate of DNA synthesis (e.g., by pulse-labeling cultures of T cells with tritiated thymidine and measuring the amount of tritiated thymidine incorporated into DNA). Contact with a tumor polypeptide (100 ng/ml-100 μg/ml, preferably 200 ng/ml-25 μg/ml) for 3-7 days will typically result in at least a two fold increase in proliferation of the T cells. Contact as described above for 2-3 hours should result in activation of the T cells, as measured using standard cytokine assays in which a two fold increase in the level of cytokine release (e.g., TNF or IFN-γ) is indicative of T cell activation (see Coligan et al., Current Protocols in Immunology, vol. 1, Wiley Interscience (Greene 1998)). T cells that have been activated in response to a tumor polypeptide, polynucleotide or polypeptide-expressing APC may be CD4⁺ and/or CD8⁺. Tumor polypeptide-specific T cells may be expanded using standard techniques. Within preferred embodiments, the T cells are derived from a patient, a related donor or an unrelated donor, and are administered to the patient following stimulation and expansion.

For therapeutic purposes, CD4⁺ or CD8⁺ T cells that proliferate in response to a tumor polypeptide, polynucleotide or APC can be expanded in number either in vitro or in vivo. Proliferation of such T cells in vitro may be accomplished in a variety of ways. For example, the T cells can be re-exposed to a tumor polypeptide, or a short peptide corresponding to an immunogenic portion of such a polypeptide, with or without the addition of T cell growth factors, such as interleukin-2, and/or stimulator cells that synthesize a tumor polypeptide. Alternatively, one or more T cells that proliferate in the presence of the tumor polypeptide can be expanded in number by cloning. Methods for cloning cells are well known in the art, and include limiting dilution.

Pharmaceutical Compositions In additional embodiments, the present invention concerns formulation of one or more of the polynucleotide, polypeptide, T-cell and/or antibody compositions disclosed herein in pharmaceutically-acceptable carriers for administration to a cell or an animal, either alone, or in combination with one or more other modalities of therapy.

It will be understood that, if desired, a composition as disclosed herein may be administered in combination with other agents as well, such as, e.g., other proteins or polypeptides or various pharmaceutically-active agents. In fact, there is virtually no limit to other components that may also be included, given that the additional agents do not cause a significant adverse effect upon contact with the target cells or host tissues. The compositions may thus be delivered along with various other agents as required in the particular instance. Such compositions may be purified from host cells or other biological sources, or alternatively may be chemically synthesized as described herein. Likewise, such compositions may further comprise substituted or derivatized RNA or DNA compositions.

Therefore, in another aspect of the present invention, pharmaceutical compositions are provided comprising one or more of the polynucleotide, polypeptide, antibody, and/or T-cell compositions described herein in combination with a physiologically acceptable carrier. In certain preferred embodiments, the pharmaceutical compositions of the invention comprise immunogenic polynucleotide and/or polypeptide compositions of the invention for use in prophylactic and theraputic vaccine applications. Vaccine preparation is generally described in, for example, M. F. Powell and M. J. Newman, eds., “Vaccine Design (the subunit and adjuvant approach),” Plenum Press (NY, 1995). Generally, such compositions will comprise one or more polynucleotide and/or polypeptide compositions of the present invention in combination with one or more immunostimulants.

It will be apparent that any of the pharmaceutical compositions described herein can contain pharmaceutically acceptable salts of the polynucleotides and polypeptides of the invention. Such salts can be prepared, for example, from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts).

In another embodiment, illustrative immunogenic compositions, e.g., vaccine compositions, of the present invention comprise DNA encoding one or more of the polypeptides as described above, such that the polypeptide is generated in situ. As noted above, the polynucleotide may be administered within any of a variety of delivery systems known to those of ordinary skill in the art. Indeed, numerous gene delivery techniques are well known in the art, such as those described by Rolland, Crit. Rev. Therap. Drug Carrier Systems 15:143-198, 1998, and references cited therein. Appropriate polynucleotide expression systems will, of course, contain the necessary regulatory DNA regulatory sequences for expression in a patient (such as a suitable promoter and terminating signal). Alternatively, bacterial delivery systems may involve the administration of a bacterium (such as Bacillus-Calmette-Guerrin) that expresses an immunogenic portion of the polypeptide on its cell surface or secretes such an epitope.

Therefore, in certain embodiments, polynucleotides encoding immunogenic polypeptides described herein are introduced into suitable mammalian host cells for expression using any of a number of known viral-based systems. In one illustrative embodiment, retroviruses provide a convenient and effective platform for gene delivery systems. A selected nucleotide sequence encoding a polypeptide of the present invention can be inserted into a vector and packaged in retroviral particles using techniques known in the art. The recombinant virus can then be isolated and delivered to a subject. A number of illustrative retroviral systems have been described (e.g., U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Bums et al. (1993) Proc. Natl. Acad. Sci. USA 90:8033-8037; and Boris-Lawrie and Temin (1993) Cur. Opin. Genet. Develop. 3:102-109.

In addition, a number of illustrative adenovirus-based systems have also been described. Unlike retroviruses which integrate into the host genome, adenoviruses persist extrachromosomally thus minimizing the risks associated with insertional mutagenesis (Haj-Ahmad and Graham (1986) J. Virol. 57:267-274; Bett et al. (1993) J. Virol. 67:5911-5921; Mittereder et al. (1994) Human Gene Therapy 5:717-729; Seth et al. (1994) J. Virol. 68:933-940; Barr et al. (1994) Gene Therapy 1:51-58; Berkner, K. L. (1988) BioTechniques 6:616-629; and Rich et al. (1993) Human Gene Therapy 4:461-476).

Various adeno-associated virus (AAV) vector systems have also been developed for polynucleotide delivery. AAV vectors can be readily constructed using techniques well known in the art. See, e.g., U.S. Pat. Nos. 5,173,414 and 5,139,941; International Publication Nos. WO 92/01070 and WO 93/03769; Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; Kotin, R. M. (1994) Human Gene Therapy 5:793-801; Shelling and Smith (1994) Gene Therapy 1:165-169; and Zhou et al. (1994) J. Exp. Med. 179:1867-1875.

Additional viral vectors useful for delivering the polynucleotides encoding polypeptides of the present invention by gene transfer include those derived from the pox family of viruses, such as vaccinia virus and avian poxvirus. By way of example, vaccinia virus recombinants expressing the novel molecules can be constructed as follows. The DNA encoding a polypeptide is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the polypeptide of interest into the viral genome. The resulting TK.sup.(-) recombinant can be selected by culturing the cells in the presence of 5-bromodeoxyuridine and picking viral plaques resistant thereto.

A vaccinia-based infection/transfection system can be conveniently used to provide for inducible, transient expression or coexpression of one or more polypeptides described herein in host cells of an organism. In this particular system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the polynucleotide or polynucleotides of interest, driven by a T7 promoter. The polymerase expressed in the cytoplasm from the vaccinia virus recombinant transcribes the transfected DNA into RNA which is then translated into polypeptide by the host translational machinery. The method provides for high level, transient, cytoplasmic production of large quantities of RNA and its translation products. See, e.g., Elroy-Stein and Moss, Proc. Natl. Acad. Sci. USA (1990) 87:6743-6747; Fuerst et al. Proc. Natl. Acad. Sci. USA (1986) 83:8122-8126.

Alternatively, avipoxviruses, such as the fowlpox and canarypox viruses, can also be used to deliver the coding sequences of interest. Recombinant avipox viruses, expressing immunogens from mammalian pathogens, are known to confer protective immunity when administered to non-avian species. The use of an Avipox vector is particularly desirable in human and other mammalian species since members of the Avipox genus can only productively replicate in susceptible avian species and therefore are not infective in mammalian cells. Methods for producing recombinant Avipoxviruses are known in the art and employ genetic recombination, as described above with respect to the production of vaccinia viruses. See, e.g., WO 91/12882; WO 89/03429; and WO 92/03545.

Any of a number of alphavirus vectors can also be used for delivery of polynucleotide compositions of the present invention, such as those vectors described in U.S. Pat. Nos. 5,843,723; 6,015,686; 6,008,035 and 6,015,694. Certain vectors based on Venezuelan Equine Encephalitis (VEE) can also be used, illustrative examples of which can be found in U.S. Pat. Nos. 5,505,947 and 5,643,576.

Moreover, molecular conjugate vectors, such as the adenovirus chimeric vectors described in Michael et al. J. Biol. Chem. (1993) 268:6866-6869 and Wagner et al. Proc. Natl. Acad. Sci. USA (1992) 89:6099-6103, can also be used for gene delivery under the invention.

Additional illustrative information on these and other known viral-based delivery systems can be found, for example, in Fisher-Hoch et al., Proc. Natl. Acad. Sci. USA 86:317-321, 1989; Flexner et al., Ann. N.Y. Acad. Sci. 569:86-103, 1989; Flexner et al., Vaccine 8:17-21, 1990; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6:616-627, 1988; Rosenfeld et al., Science 252:431-434, 1991; Kolls et al., Proc. Natl. Acad. Sci. USA 91:215-219, 1994; Kass-Eisler et al., Proc. Natl. Acad. Sci. USA 90:11498-11502, 1993; Guzman et al., Circulation 88:2838-2848, 1993; and Guzman et al., Cir. Res. 73:1202-1207, 1993.

In certain embodiments, a polynucleotide may be integrated into the genome of a target cell. This integration may be in the specific location and orientation via homologous recombination (gene replacement) or it may be integrated in a random, non-specific location (gene augmentation). In yet further embodiments, the polynucleotide may be stably maintained in the cell as a separate, episomal segment of DNA. Such polynucleotide segments or “episomes” encode sequences sufficient to permit maintenance and replication independent of or in synchronization with the host cell cycle. The manner in which the expression construct is delivered to a cell and where in the cell the polynucleotide remains is dependent on the type of expression construct employed.

In another embodiment of the invention, a polynucleotide is administered/delivered as “naked” DNA, for example as described in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

In still another embodiment, a composition of the present invention can be delivered via a particle bombardment approach, many of which have been described. In one illustrative example, gas-driven particle acceleration can be achieved with devices such as those manufactured by Powderject Pharmaceuticals PLC (Oxford, UK) and Powderject Vaccines Inc. (Madison, Wis.), some examples of which are described in U.S. Pat. Nos. 5,846,796; 6,010,478; 5,865,796; 5,584,807; and EP Patent No. 0500 799. This approach offers a needle-free delivery approach wherein a dry powder formulation of microscopic particles, such as polynucleotide or polypeptide particles, are accelerated to high speed within a helium gas jet generated by a hand held device, propelling the particles into a target tissue of interest.

In a related embodiment, other devices and methods that may be useful for gas-driven needle-less injection of compositions of the present invention include those provided by Bioject, Inc. (Portland, Oreg.), some examples of which are described in U.S. Pat. Nos. 4,790,824; 5,064,413; 5,312,335; 5,383,851; 5,399,163; 5,520,639 and 5,993,412.

According to another embodiment, the pharmaceutical compositions described herein will comprise one or more immunostimulants in addition to the immunogenic polynucleotide, polypeptide, antibody, T-cell and/or APC compositions of this invention. An immunostimulant refers to essentially any substance that enhances or potentiates an immune response (antibody and/or cell-mediated) to an exogenous antigen. One preferred type of immunostimulant comprises an adjuvant. Many adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Certain adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, interleukin-2,-7,-12, and other like growth factors, may also be used as adjuvants.

Within certain embodiments of the invention, the adjuvant composition is preferably one that induces an immune response predominantly of the Th1 type. High levels of Th1-type cytokines (e.g., IFN-γ, TNFα, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, Ann. Rev. Immunol. 7:145-173, 1989.

Certain preferred adjuvants for eliciting a predominantly Th1-type response include, for example, a combination of monophosphoryl lipid A, preferably 3-de-O-acylated monophosphoryl lipid A, together with an aluminum salt. MPL® adjuvants are available from Corixa Corporation (Seattle, Wash.; see, for example, US Pat. Nos. 4,436,727; 4,877,611; 4,866,034 and 4,912,094). CpG-containing oligonucleotides (in which the CpG dinucleotide is unrethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996. Another preferred adjuvant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins. Other preferred formulations include more than one saponin in the adjuvant combinations of the present invention, for example combinations of at least two of the following group comprising QS21, QS7, Quil A, β-escin, or digitonin.

Alternatively the saponin formulations may be combined with vaccine vehicles composed of chitosan or other polycationic polymers, polylactide and polylactide-co-glycolide particles, poly-N-acetyl glucosamine-based polymer matrix, particles composed of polysaccharides or chemically modified polysaccharides, liposomes and lipid-based particles, particles composed of glycerol monoesters, etc. The saponins may also be formulated in the presence of cholesterol to form particulate structures such as liposomes or ISCOMs. Furthermore, the saponins may be formulated together with a polyoxyethylene ether or ester, in either a non-particulate solution or suspension, or in a particulate structure such as a paucilamelar liposome or ISCOM. The saponins may also be formulated with excipients such as Carbopol^{R} to increase viscosity, or may be formulated in a dry powder form with a powder excipient such as lactose.

In one preferred embodiment, the adjuvant system includes the combination of a monophosphoryl lipid A and a saponin derivative, such as the combination of QS21 and 3D-MPL® adjuvant, as described in WO 94/00153, or a less reactogenic composition where the QS21 is quenched with cholesterol, as described in WO 96/33739. Other preferred formulations comprise an oil-in-water emulsion and tocopherol. Another particularly preferred adjuvant formulation employing QS21, 3D-MPL® adjuvant and tocopherol in an oil-in-water emulsion is described in WO 95/17210.

Another enhanced adjuvant system involves the combination of a CpG-containing oligonucleotide and a saponin derivative particularly the combination of CpG and QS21 is disclosed in WO 00/09159. Preferably the formulation additionally comprises an oil in water emulsion and tocopherol.

Additional illustrative adjuvants for use in the pharmaceutical compositions of the invention include Montanide ISA 720 (Seppic, France), SAF (Chiron, Calif., United States), ISCOMS (CSL), MF-59 (Chiron), the SBAS series of adjuvants (e.g., SBAS-2 or SBAS-4, available from SmithKline Beecham, Rixensart, Belgium), Detox (Enhanzyn®) (Corixa, Hamilton, Mont.), RC-529 (Corixa, Hamilton, Mont.) and other aminoalkyl glucosaminide 4-phosphates (AGPs), such as those described in pending U.S. patent application Ser. Nos. 08/853,826 and 09/074,720, the disclosures of which are incorporated herein by reference in their entireties, and polyoxyethylene ether adjuvants such as those described in WO 99/52549A1.

Other preferred adjuvants include adjuvant molecules of the general formula

HO(CH₂CH₂O)ₙ—A—R, (I)

wherein, n is 1-50, A is a bond or —C(O)—, R is C₁₋₅₀ alkyl or Phenyl C₁₋₅₀ alkyl.

One embodiment of the present invention consists of a vaccine formulation comprising a polyoxyethylene ether of general formula (I), wherein n is between 1 and 50, preferably 4-24, most preferably 9; the R component is C₁₋₅₀, preferably C₄-C₂₀ alkyl and most preferably C₁₂ alkyl, and A is a bond. The concentration of the polyoxyethylene ethers should be in the range 0.1-20%, preferably from 0.1-10%, and most preferably in the range 0.1-1%. Preferred polyoxyethylene ethers are selected from the following group: polyoxyethylene-9-lauryl ether, polyoxyethylene-9-steoryl ether, polyoxyethylene-8-steoryl ether, polyoxyethylene-4-lauryl ether, polyoxyethylene-35-lauryl ether, and polyoxyethylene-23-lauryl ether. Polyoxyethylene ethers such as polyoxyethylene lauryl ether are described in the Merck index (12^{th} edition: entry 7717). These adjuvant molecules are described in WO 99/52549.

The polyoxyethylene ether according to the general formula (I) above may, if desired, be combined with another adjuvant. For example, a preferred adjuvant combination is preferably with CpG as described in the pending UK patent application GB 9820956.2.

According to another embodiment of this invention, an immunogenic composition described herein is delivered to a host via antigen presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-tumor effects per se and/or to be immunologically compatible with the receiver (i.e., matched HLA haplotype). APCs may generally be isolated from any of a variety of biological fluids and organs, including tumor and peritumoral tissues, and may be autologous, allogeneic, syngeneic or xenogeneic cells.

Certain preferred embodiments of the present invention use dendritic cells or progenitors thereof as antigen-presenting cells. Dendritic cells are highly potent APCs (Banchereau and Steinman, Nature 392:245-251, 1998) and have been shown to be effective as a physiological adjuvant for eliciting prophylactic or therapeutic antitumor immunity (see Timmerman and Levy, Ann. Rev. Med. 50:507-529, 1999). In general, dendritic cells may be identified based on their typical shape (stellate in situ, with marked cytoplasmic processes (dendrites) visible in vitro), their ability to take up, process and present antigens with high efficiency and their ability to activate naive T cell responses. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within a vaccine (see Zitvogel et al., Nature Med. 4:594-600, 1998).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, flt3 ligand and/or other compound(s) that induce differentiation, maturation and proliferation of dendritic cells.

Dendritic cells are conveniently categorized as “immature” and “mature” cells, which allows a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fcγ receptor and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1BB).

APCs may generally be transfected with a polynucleotide of the invention (or portion or other variant thereof) such that the encoded polypeptide, or an immunogenic portion thereof, is expressed on the cell surface. Such transfection may take place ex vivo, and a pharmaceutical composition comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., Immunology and cell Biology 75:456-460, 1997. Antigen loading of dendritic cells may be achieved by incubating dendritic cells or progenitor cells with the tumor polypeptide, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule). Alternatively, a dendritic cell may be pulsed with a non-conjugated immunological partner, separately or in the presence of the polypeptide.

While any suitable carrier known to those of ordinary skill in the art may be employed in the pharmaceutical compositions of this invention, the type of carrier will typically vary depending on the mode of administration. Compositions of the present invention may be formulated for any appropriate manner of administration, including for example, topical, oral, nasal, mucosal, intravenous, intracranial, intraperitoneal, subcutaneous and intramuscular administration.

Carriers for use within such pharmaceutical compositions are biocompatible, and may also be biodegradable. In certain embodiments, the formulation preferably provides a relatively constant level of active component release. In other embodiments, however, a more rapid rate of release immediately upon administration may be desired. The formulation of such compositions is well within the level of ordinary skill in the art using known techniques. Illustrative carriers useful in this regard include microparticles of poly(lactide-co-glycolide), polyacrylate, latex, starch, cellulose, dextran and the like. Other illustrative delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see e.g., U.S. Pat. No. 5,151,254 and PCT applications WO 94/20078, WO/94/23701 and WO 96/06638). The amount of active compound contained within a sustained release formulation depends upon the site of implantation, the rate and expected duration of release and the nature of the condition to be treated or prevented.

In another illustrative embodiment, biodegradable microspheres (e.g., polylactate polyglycolate) are employed as carriers for the compositions of this invention. Suitable biodegradable microspheres are disclosed, for example, in U.S. Pat. Nos.4,897,268; 5,075,109; 5,928,647; 5,811,128; 5,820,883; 5,853,763; 5,814,344, 5,407,609 and 5,942,252. Modified hepatitis B core protein carrier systems such as described in WO/99 40934, and references cited therein, will also be useful for many applications. Another illustrative carrier/delivery system employs a carrier comprising particulate-protein complexes, such as those described in U.S. Pat. No. 5,928,647, which are capable of inducing a class I-restricted cytotoxic T lymphocyte responses in a host.

The pharmaceutical compositions of the invention will often further comprise one or more buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate.

The pharmaceutical compositions described herein may be presented in unit-dose or multi-dose containers, such as sealed ampoules or vials. Such containers are typically sealed in such a way to preserve the sterility and stability of the formulation until use. In general, formulations may be stored as suspensions, solutions or emulsions in oily or aqueous vehicles. Alternatively, a pharmaceutical composition may be stored in a freeze-dried condition requiring only the addition of a sterile liquid carrier immediately prior to use.

The development of suitable dosing and treatment regimens for using the particular compositions described herein in a variety of treatment regimens, including e.g., oral, parenteral, intravenous, intranasal, and intramuscular administration and formulation, is well known in the art, some of which are briefly discussed below for general purposes of illustration.

In certain applications, the pharmaceutical compositions disclosed herein may be delivered via oral administration to an animal. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

The active compounds may even be incorporated with excipients and used in the form of ingestible tablets, buccal tables, troches, capsules, elixirs, suspensions, syrups, wafers, and the like (see, for example, Mathiowitz et al., Nature 1997 Mar 27;386(6623):410-4; Hwang et al., Crit Rev Ther Drug Carrier Syst 1998;15(3):243-84; U.S. Pat. No. 5,641,515; U.S. Pat. No. 5,580,579 and U.S. Pat. No. 5,792,451). Tablets, troches, pills, capsules and the like may also contain any of a variety of additional components, for example, a binder, such as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Of course, any material used in preparing any dosage unit form should be pharmaceutically pure and substantially non-toxic in the amounts employed. In addition, the active compounds may be incorporated into sustained-release preparation and formulations.

Typically, these formulations will contain at least about 0.1% of the active compound or more, although the percentage of the active ingredient(s) may, of course, be varied and may conveniently be between about 1 or 2% and about 60% or 70% or more of the weight or volume of the total formulation. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

For oral administration the compositions of the present invention may alternatively be incorporated with one or more excipients in the form of a mouthwash, dentifrice, buccal tablet, oral spray, or sublingual orally-administered formulation. Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

In certain circumstances it will be desirable to deliver the pharmaceutical compositions disclosed herein parenterally, intravenously, intramuscularly, or even intraperitoneally. Such approaches are well known to the skilled artisan, some of which are further described, for example, in U.S. Pat. No. 5,543,158; U.S. Pat. No. 5,641,515 and U.S. Pat. No. 5,399,363. In certain embodiments, solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations generally will contain a preservative to prevent the growth of microorganisms.

Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and/or by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In one embodiment, for parenteral administration in an aqueous solution, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. Moreover, for human administration, preparations will of course preferably meet sterility, pyrogenicity, and the general safety and purity standards as required by FDA Office of Biologics standards.

In another embodiment of the invention, the compositions disclosed herein may be formulated in a neutral or salt form. Illustrative pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective.

The carriers can further comprise any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically-acceptable” refers to molecular entities and compositions that do not produce an allergic or similar untoward reaction when administered to a human.

In certain embodiments, the pharmaceutical compositions may be delivered by intranasal sprays, inhalation, and/or other aerosol delivery vehicles. Methods for delivering genes, nucleic acids, and peptide compositions directly to the lungs via nasal aerosol sprays has been described, e.g., in U.S. Pat. No. 5,756,353 and U.S. Pat. No. 5,804,212. Likewise, the delivery of drugs using intranasal microparticle resins (Takenaga et al., J Controlled Release 1998 Mar 2;52(1-2):81-7) and lysophosphatidyl-glycerol compounds (U.S. Pat. No. 5,725,871) are also well-known in the pharmaceutical arts. Likewise, illustrative transmucosal drug delivery in the form of a polytetrafluoroetheylene support matrix is described in U.S. Pat. No. 5,780,045.

In certain embodiments, liposomes, nanocapsules, microparticles, lipid particles, vesicles, and the like, are used for the introduction of the compositions of the present invention into suitable host cells/organisms. In particular, the compositions of the present invention may be formulated for delivery either encapsulated in a lipid particle, a liposome, a vesicle, a nanosphere, or a nanoparticle or the like. Alternatively, compositions of the present invention can be bound, either covalently or non-covalently, to the surface of such carrier vehicles.

The formation and use of liposome and liposome-like preparations as potential drug carriers is generally known to those of skill in the art (see for example, Lasic, Trends Biotechnol 1998 Jul;16(7):307-21; Takakura, Nippon Rinsho 1998 Mar;56(3):691-5; Chandran et al., Indian J Exp Biol. 1997 Aug;35(8):801-9; Margalit, Crit Rev Ther Drug Carrier Syst. 1995;12(2-3):233-61; U.S. Pat. No. 5,567,434; U.S. Pat. No. 5,552,157; U.S. Pat. No. 5,565,213; U.S. Pat. No. 5,738,868 and U.S. Pat. No. 5,795,587, each specifically incorporated herein by reference in its entirety).

Liposomes have been used successfully with a number of cell types that are normally difficult to transfect by other procedures, including T cell suspensions, primary hepatocyte cultures and PC 12 cells (Renneisen et al., J Biol Chem. 1990 Sep 25;265(27):16337-42; Muller et al., DNA Cell Biol. 1990 Apr;9(3):221-9). In addition, liposomes are free of the DNA length constraints that are typical of viral-based delivery systems. Liposomes have been used effectively to introduce genes, various drugs, radiotherapeutic agents, enzymes, viruses, transcription factors, allosteric effectors and the like, into a variety of cultured cell lines and animals. Furthermore, he use of liposomes does not appear to be associated with autoimmune responses or unacceptable toxicity after systemic delivery.

In certain embodiments, liposomes are formed from phospholipids that are dispersed in an aqueous medium and spontaneously form multilamellar concentric bilayer vesicles (also termed multilamellar vesicles (MLVs).

Alternatively, in other embodiments, the invention provides for pharmaceutically-acceptable nanocapsule formulations of the compositions of the present invention. Nanocapsules can generally entrap compounds in a stable and reproducible way (see, for example, Quintanar-Guerrero et al., Drug Dev Ind Pharm. 1998 Dec;24(12):1113-28). To avoid side effects due to intracellular polymeric overloading, such ultrafine particles (sized around 0.1 μm) may be designed using polymers able to be degraded in vivo. Such particles can be made as described, for example, by Couvreur et al., Crit Rev Ther Drug Carrier Syst. 1988;5(1):1-20; Eur Muhlen et al., Eur J Pharm Biopharm. 1998 Mar;45(2):149-55; Zambaux et al. J Controlled Release. 1998 Jan 2;50(1-3):31-40; and U.S. Pat. No. 5,145,684.

Cancer Therapeutic Methods

In further aspects of the present invention, the pharmaceutical compositions described herein may be used for the treatment of cancer, particularly for the immunotherapy of colon cancer. Within such methods, the pharmaceutical compositions described herein are administered to a patient, typically a warm-blooded animal, preferably a human. A patient may or may not be afflicted with cancer. Accordingly, the above pharmaceutical compositions may be used to prevent the development of a cancer or to treat a patient afflicted with a cancer. Pharmaceutical compositions and vaccines may be administered either prior to or following surgical removal of primary tumors and/or treatment such as administration of radiotherapy or conventional chemotherapeutic drugs. As discussed above, administration of the pharmaceutical compositions may be by any suitable method, including administration by intravenous, intraperitoneal, intramuscular, subcutaneous, intranasal, intradermal, anal, vaginal, topical and oral routes.

Within certain embodiments, immunotherapy may be active immunotherapy, in which treatment relies on the in vivo stimulation of the endogenous host immune system to react against tumors with the administration of immune response-modifying agents (such as polypeptides and polynucleotides as provided herein).

Within other embodiments, immunotherapy may be passive immunotherapy, in which treatment involves the delivery of agents with established tumor-immune reactivity (such as effector cells or antibodies) that can directly or indirectly mediate antitumor effects and does not necessarily depend on an intact host immune system. Examples of effector cells include T cells as discussed above, T lymphocytes (such as CD8⁺ cytotoxic T lymphocytes and CD4⁺ T-helper tumor-infiltrating lymphocytes), killer cells (such as Natural Killer cells and lymphokine-activated killer cells), B cells and antigen-presenting cells (such as dendritic cells and macrophages) expressing a polypeptide provided herein. T cell receptors and antibody receptors specific for the polypeptides recited herein may be cloned, expressed and transferred into other vectors or effector cells for adoptive immunotherapy. The polypeptides provided herein may also be used to generate antibodies or anti-idiotypic antibodies (as described above and in U.S. Pat. No. 4,918,164) for passive immunotherapy.

Effector cells may generally be obtained in sufficient quantities for adoptive immunotherapy by growth in vitro, as described herein. Culture conditions for expanding single antigen-specific effector cells to several billion in number with retention of antigen recognition in vivo are well known in the art. Such in vitro culture conditions typically use intermittent stimulation with antigen, often in the presence of cytokines (such as IL-2) and non-dividing feeder cells. As noted above, immunoreactive polypeptides as provided herein may be used to rapidly expand antigen-specific T cell cultures in order to generate a sufficient number of cells for immunotherapy. In particular, antigen-presenting cells, such as dendritic, macrophage, monocyte, fibroblast and/or B cells, may be pulsed with immunoreactive polypeptides or transfected with one or more polynucleotides using standard techniques well known in the art. For example, antigen-presenting cells can be transfected with a polynucleotide having a promoter appropriate for increasing expression in a recombinant virus or other expression system. Cultured effector cells for use in therapy must be able to grow and distribute widely, and to survive long term in vivo. Studies have shown that cultured effector cells can be induced to grow in vivo and to survive long term in substantial numbers by repeated stimulation with antigen supplemented with IL-2 (see, for example, Cheever et al., Immunological Reviews 157:177, 1997).

Alternatively, a vector expressing a polypeptide recited herein may be introduced into antigen presenting cells taken from a patient and clonally propagated ex vivo for transplant back into the same patient. Transfected cells may be reintroduced into the patient using any means known in the art, preferably in sterile form by intravenous, intracavitary, intraperitoneal or intratumor administration.

Routes and frequency of administration of the therapeutic compositions described herein, as well as dosage, will vary from individual to individual, and may be readily established using standard techniques. In general, the pharmaceutical compositions and vaccines may be administered by injection (e.g., intracutaneous, intramuscular, intravenous or subcutaneous), intranasally (e.g., by aspiration) or orally. Preferably, between 1 and 10 doses may be administered over a 52 week period. Preferably, 6 doses are administered, at intervals of 1 month, and booster vaccinations may be given periodically thereafter. Alternate protocols may be appropriate for individual patients. A suitable dose is an amount of a compound that, when administered as described above, is capable of promoting an anti-tumor immune response, and is at least 10-50% above the basal (i.e., untreated) level. Such response can be monitored by measuring the anti-tumor antibodies in a patient or by vaccine-dependent generation of cytolytic effector cells capable of killing the patient's tumor cells in vitro. Such vaccines should also be capable of causing an immune response that leads to an improved clinical outcome (e.g., more frequent remissions, complete or partial or longer disease-free survival) in vaccinated patients as compared to non-vaccinated patients. In general, for pharmaceutical compositions and vaccines comprising one or more polypeptides, the amount of each polypeptide present in a dose ranges from about 25 μg to 5 mg per kg of host. Suitable dose sizes will vary with the size of the patient, but will typically range from about 0.1 mL to about 5 mL.

In general, an appropriate dosage and treatment regimen provides the active compound(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients. Increases in preexisting immune responses to a tumor protein generally correlate with an improved clinical outcome. Such immune responses may generally be evaluated using standard proliferation, cytotoxicity or cytokine assays, which may be performed using samples obtained from a patient before and after treatment.

Cancer Detection and Diagnostic Compositions, Methods and Kits

In general, a cancer may be detected in a patient based on the presence of one or more colon tumor proteins and/or polynucleotides encoding such proteins in a biological sample (for example, blood, sera, sputum urine and/or tumor biopsies) obtained from the patient. In other words, such proteins may be used as markers to indicate the presence or absence of a cancer such as colon cancer. In addition, such proteins may be useful for the detection of other cancers. The binding agents provided herein generally permit detection of the level of antigen that binds to the agent in the biological sample. Polynucleotide primers and probes may be used to detect the level of mRNA encoding a tumor protein, which is also indicative of the presence or absence of a cancer. In general, a colon tumor sequence should be present at a level that is at least three fold higher in tumor tissue than in normal tissue.

There are a variety of assay formats known to those of ordinary skill in the art for using a binding agent to detect polypeptide markers in a sample. See, e.g., Harlow and Lane, Antibodies: A Laboratory Manual, Cold Spring Harbor Laboratory, 1988. In general, the presence or absence of a cancer in a patient may be determined by (a) contacting a biological sample obtained from a patient with a binding agent; (b) detecting in the sample a level of polypeptide that binds to the binding agent; and (c) comparing the level of polypeptide with a predetermined cut-off value.

In a preferred embodiment, the assay involves the use of binding agent immobilized on a solid support to bind to and remove the polypeptide from the remainder of the sample. The bound polypeptide may then be detected using a detection reagent that contains a reporter group and specifically binds to the binding agent/polypeptide complex. Such detection reagents may comprise, for example, a binding agent that specifically binds to the polypeptide or an antibody or other agent that specifically binds to the binding agent, such as an anti-immunoglobulin, protein G, protein A or a lectin. Alternatively, a competitive assay may be utilized, in which a polypeptide is labeled with a reporter group and allowed to bind to the immobilized binding agent after incubation of the binding agent with the sample. The extent to which components of the sample inhibit the binding of the labeled polypeptide to the binding agent is indicative of the reactivity of the sample with the immobilized binding agent. Suitable polypeptides for use within such assays include full length colon tumor proteins and polypeptide portions thereof to which the binding agent binds, as described above.

The solid support may be any material known to those of ordinary skill in the art to which the tumor protein may be attached. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681. The binding agent may be immobilized on the solid support using a variety of techniques known to those of skill in the art, which are amply described in the patent and scientific literature. In the context of the present invention, the term “immobilization” refers to both noncovalent association, such as adsorption, and covalent attachment (which may be a direct linkage between the agent and functional groups on the support or may be a linkage by way of a cross-linking agent). Immobilization by adsorption to a well in a microtiter plate or to a membrane is preferred. In such cases, adsorption may be achieved by contacting the binding agent, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of binding agent ranging from about 10 ng to about 10 μg, and preferably about 100 ng to about 1 μg, is sufficient to immobilize an adequate amount of binding agent.

Covalent attachment of binding agent to a solid support may generally be achieved by first reacting the support with a bifunctional reagent that will react with both the support and a functional group, such as a hydroxyl or amino group, on the binding agent. For example, the binding agent may be covalently attached to supports having an appropriate polymer coating using benzoquinone or by condensation of an aldehyde group on the support with an amine and an active hydrogen on the binding partner (see, e.g., Pierce Immunotechnology Catalog and Handbook, 1991, at A12-A13).

In certain embodiments, the assay is a two-antibody sandwich assay. This assay may be performed by first contacting an antibody that has been immobilized on a solid support, commonly the well of a microtiter plate, with the sample, such that polypeptides within the sample are allowed to bind to the immobilized antibody. Unbound sample is then removed from the immobilized polypeptide-antibody complexes and a detection reagent (preferably a second antibody capable of binding to a different site on the polypeptide) containing a reporter group is added. The amount of detection reagent that remains bound to the solid support is then determined using a method appropriate for the specific reporter group.

More specifically, once the antibody is immobilized on the support as described above, the remaining protein binding sites on the support are typically blocked. Any suitable blocking agent known to those of ordinary skill in the art, such as bovine serum albumin or Tween 20™ (Sigma Chemical Co., St. Louis, Mo.). The immobilized antibody is then incubated with the sample, and polypeptide is allowed to bind to the antibody. The sample may be diluted with a suitable diluent, such as phosphate-buffered saline (PBS) prior to incubation. In general, an appropriate contact time (i.e., incubation time) is a period of time that is sufficient to detect the presence of polypeptide within a sample obtained from an individual with colon cancer. Preferably, the contact time is sufficient to achieve a level of binding that is at least about 95% of that achieved at equilibrium between bound and unbound polypeptide. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% Tween 20™. The second antibody, which contains a reporter group, may then be added to the solid support. Preferred reporter groups include those groups recited above.

The detection reagent is then incubated with the immobilized antibody-polypeptide complex for an amount of time sufficient to detect the bound polypeptide. An appropriate amount of time may generally be determined by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of a cancer, such as colon cancer, the signal detected from the reporter group that remains bound to the solid support is generally compared to a signal that corresponds to a predetermined cut-off value. In one preferred embodiment, the cut-off value for the detection of a cancer is the average mean signal obtained when the immobilized antibody is incubated with samples from patients without the cancer. In general, a sample generating a signal that is three standard deviations above the predetermined cut-off value is considered positive for the cancer. In an alternate preferred embodiment, the cut-off value is determined using a Receiver Operator Curve, according to the method of Sackett et al., Clinical Epidemiology: A Basic Science for Clinical Medicine, Little Brown and Co., 1985, p. 106-7. Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for a cancer.

In a related embodiment, the assay is performed in a flow-through or strip test format, wherein the binding agent is immobilized on a membrane, such as nitrocellulose. In the flow-through test, polypeptides within the sample bind to the immobilized binding agent as the sample passes through the membrane. A second, labeled binding agent then binds to the binding agent-polypeptide complex as a solution containing the second binding agent flows through the membrane. The detection of bound second binding agent may then be performed as described above. In the strip test format, one end of the membrane to which binding agent is bound is immersed in a solution containing the sample. The sample migrates along the membrane through a region containing second binding agent and to the area of immobilized binding agent. Concentration of second binding agent at the area of immobilized antibody indicates the presence of a cancer. Typically, the concentration of second binding agent at that site generates a pattern, such as a line, that can be read visually. The absence of such a pattern indicates a negative result. In general, the amount of binding agent immobilized on the membrane is selected to generate a visually discernible pattern when the biological sample contains a level of polypeptide that would be sufficient to generate a positive signal in the two-antibody sandwich assay, in the format discussed above. Preferred binding agents for use in such assays are antibodies and antigen-binding fragments thereof. Preferably, the amount of antibody immobilized on the membrane ranges from about 25 ng to about 1 μg, and more preferably from about 50 ng to about 500 ng. Such tests can typically be performed with a very small amount of biological sample.

Of course, numerous other assay protocols exist that are suitable for use with the tumor proteins or binding agents of the present invention. The above descriptions are intended to be exemplary only. For example, it will be apparent to those of ordinary skill in the art that the above protocols may be readily modified to use tumor polypeptides to detect antibodies that bind to such polypeptides in a biological sample. The detection of such tumor protein specific antibodies may correlate with the presence of a cancer.

A cancer may also, or alternatively, be detected based on the presence of T cells that specifically react with a tumor protein in a biological sample. Within certain methods, a biological sample comprising CD4⁺ and/or CD8⁺ T cells isolated from a patient is incubated with a tumor polypeptide, a polynucleotide encoding such a polypeptide and/or an APC that expresses at least an immunogenic portion of such a polypeptide, and the presence or absence of specific activation of the T cells is detected. Suitable biological samples include, but are not limited to, isolated T cells. For example, T cells may be isolated from a patient by routine techniques (such as by Ficoll/Hypaque density gradient centrifugation of peripheral blood lymphocytes). T cells may be incubated in vitro for 2-9 days (typically 4 days) at 37° C. with polypeptide (e.g., 5-25 μg/ml). It may be desirable to incubate another aliquot of a T cell sample in the absence of colontumor polypeptide to serve as a control. For CD4⁺ T cells, activation is preferably detected by evaluating proliferation of the T cells. For CD8⁺ T cells, activation is preferably detected by evaluating cytolytic activity. A level of proliferation that is at least two fold greater and/or a level of cytolytic activity that is at least 20% greater than in disease-free patients indicates the presence of a cancer in the patient.

As noted above, a cancer may also, or alternatively, be detected based on the level of mRNA encoding a colontumor protein in a biological sample. For example, at least two oligonucleotide primers may be employed in a polymerase chain reaction (PCR) based assay to amplify a portion of a tumor cDNA derived from a biological sample, wherein at least one of the oligonucleotide primers is specific for (i.e., hybridizes to) a polynucleotide encoding the tumor protein. The amplified cDNA is then separated and detected using techniques well known in the art, such as gel electrophoresis. Similarly, oligonucleotide probes that specifically hybridize to a polynucleotide encoding a tumor protein may be used in a hybridization assay to detect the presence of polynucleotide encoding the tumor protein in a biological sample.

To permit hybridization under assay conditions, oligonucleotide primers and probes should comprise an oligonucleotide sequence that has at least about 60%, preferably at least about 75% and more preferably at least about 90%, identity to a portion of a polynucleotide encoding a tumor protein of the invention that is at least 10 nucleotides, and preferably at least 20 nucleotides, in length. Preferably, oligonucleotide primers and/or probes hybridize to a polynucleotide encoding a polypeptide described herein under moderately stringent conditions, as defined above. Oligonucleotide primers and/or probes which may be usefully employed in the diagnostic methods described herein preferably are at least 10-40 nucleotides in length. In a preferred embodiment, the oligonucleotide primers comprise at least 10 contiguous nucleotides, more preferably at least 15 contiguous nucleotides, of a DNA molecule having a sequence as disclosed herein. Techniques for both PCR based assays and hybridization assays are well known in the art (see, for example, Mullis et al., Cold Spring Harbor Symp. Quant. Biol., 51:263, 1987; Erlich ed., PCR Technology, Stockton Press, N.Y., 1989).

One preferred assay employs RT-PCR, in which PCR is applied in conjunction with reverse transcription. Typically, RNA is extracted from a biological sample, such as biopsy tissue, and is reverse transcribed to produce cDNA molecules. PCR amplification using at least one specific primer generates a cDNA molecule, which may be separated and visualized using, for example, gel electrophoresis. Amplification may be performed on biological samples taken from a test patient and from an individual who is not afflicted with a cancer. The amplification reaction may be performed on several dilutions of cDNA spanning two orders of magnitude. A two-fold or greater increase in expression in several dilutions of the test patient sample as compared to the same dilutions of the non-cancerous sample is typically considered positive.

In another embodiment, the compositions described herein may be used as markers for the progression of cancer. In this embodiment, assays as described above for the diagnosis of a cancer may be performed over time, and the change in the level of reactive polypeptide(s) or polynucleotide(s) evaluated. For example, the assays may be performed every 24-72 hours for a period of 6 months to 1 year, and thereafter performed as needed. In general, a cancer is progressing in those patients in whom the level of polypeptide or polynucleotide detected increases over time. In contrast, the cancer is not progressing when the level of reactive polypeptide or polynucleotide either remains constant or decreases with time.

Certain in vivo diagnostic assays may be performed directly on a tumor. One such assay involves contacting tumor cells with a binding agent. The bound binding agent may then be detected directly or indirectly via a reporter group. Such binding agents may also be used in histological applications. Alternatively, polynucleotide probes may be used within such applications.

As noted above, to improve sensitivity, multiple tumor protein markers may be assayed within a given sample. It will be apparent that binding agents specific for different proteins provided herein may be combined within a single assay. Further, multiple primers or probes may be used concurrently. The selection of tumor protein markers may be based on routine experiments to determine combinations that results in optimal sensitivity. In addition, or alternatively, assays for tumor proteins provided herein may be combined with assays for other known tumor antigens.

The present invention further provides kits for use within any of the above diagnostic methods. Such kits typically comprise two or more components necessary for performing a diagnostic assay. Components may be compounds, reagents, containers and/or equipment. For example, one container within a kit may contain a monoclonal antibody or fragment thereof that specifically binds to a tumor protein. Such antibodies or fragments may be provided attached to a support material, as described above. One or more additional containers may enclose elements, such as reagents or buffers, to be used in the assay. Such kits may also, or alternatively, contain a detection reagent as described above that contains a reporter group suitable for direct or indirect detection of antibody binding.

Alternatively, a kit may be designed to detect the level of mRNA encoding a tumor protein in a biological sample. Such kits generally comprise at least one oligonucleotide probe or primer, as described above, that hybridizes to a polynucleotide encoding a tumor protein. Such an oligonucleotide may be used, for example, within a PCR or hybridization assay. Additional components that may be present within such kits include a second oligonucleotide and/or a diagnostic reagent or container to facilitate the detection of a polynucleotide encoding a tumor protein.

The following Examples are offered by way of illustration and not by way of limitation.

### EXAMPLES

### Example 1

### Identification Of Colon Tumor Protein cDNAs

This Example illustrates the identification of cDNA molecules differentially expressed in colon tumors versus normal tissues.

A colon tumor cell line subtracted library (CTLS1) was generated by conventional, biotin-streptavidin subtraction. Briefly, 10 μg plasmid DNA from the colon tumor cell line 391-12 library (754-1) was subtracted against 100 μg biotinylated driver [25% normal colon library, 25% normal liver and salivary gland library, and 50% pooled driver library (liver, pancreas, skin, bone marrow, resting PBMC, stomach, and whole brain)]. Two biotin-streptavidin subtractions were performed, one after an overnight hybridization and one after a 2-hour hybridization. cDNA remaining after the two subtractions was ligated into a Not I-cut pcDNA3.1 (+) vector, electroporated into ElectroMAX DH10B cells, and grown on agar plates containing ampicillin. Clones were randomly selected for individual amplification. Turbo miniprep DNA was prepared from each clone and characterized by sequencing and database analysis.

A total of 1248 clones from the CTLS1 conventional subtraction library were analyzed on Colon Chip 4 using cDNA microarray technology, essentially as described in Shena et al.(Shena, M. et al., 1995 Science 270:467-70). Briefly, clones are arrayed onto glass slides as multiple replicas, with each location corresponding to a unique cDNA clone. 30-35 replica chips are typically hybridized with a wide variety of cDNA probe pairs, derived from tumor-specific and normal tissue cDNA sources, that are fluorescence-labeled with Cy3 and Cy5, respectively. After hybridization, the chips are scanned and the fluorescence intensities recorded for both Cy3 and Cy5 channels. The relative intensities of the Cy3 and Cy5 signals thereby provide information about the expression of the cDNA clones in tumors versus normal tissues.

From this analysis, 146 clones were sequenced based on the fact that they showed greater than 2-fold overexpression in colon tumors versus a set of normal tissues. 71 separate cDNA sequences were derived from these clones, and are set forth in SEQ ID NOs:1-71.

Additional subtracted libraries, CCLS1 and CCLS2, were made by a PCR-based subtraction method Briefly, colon tumor cell lines were established from colon adenocarcinoma tissue grown in SCID mice. cDNAs from three pools of colon tumor cell lines were synthesized and subtracted with a set of transcripts from normal lung, adrenal gland, bone marrow, small intestine, stomach, pancreas, normal colon, HMEC (human mammary epithelial cell line) and SCID mouse liver/spleen samples. For the ADPS library, cDNAs from three colon adenocarcinoma samples were synthesized and subtracted with a set of transcripts from normal lung, adrenal gland, bone marrow, small intestine, stomach, pancreas, normal colon, HMEC (human mammary epithelial cell line) and SCID mouse liver/spleen samples. 288 CCLS1 cDNA sequences, 960 CCLS2 cDNA sequences and 1344 ADPS cDNA sequences were evaluated by microarray analysis on Colon Chip 4, as described above. From this analysis, 275 cDNA sequences were identified that exhibited greater than 2-fold overexpression in colon tumors versus a comprehensive set of normal tissues. These sequences are set forth in SEQ ID NOs:72-348.

### Example 2

### Identification Of Additional Colon Tumor Protein cDNAs

Further analysis of microarray data revealed 3 additional clones that showed greater than 2-fold overexpression in colon tumors versus a set of normal tissues. These cDNAs are disclosed in SEQ ID NOs:349-351.

Additional sequence was obtained from subtraction clone inserts described in Example 2 above that were designated as prospective candidates for further analysis (disclosed in SEQ ID NOS:5, 26, 28, 29, 34, 41, 50, 52, 54, 60, 65, and 69). The corresponding cDNA sequences with the additional sequence of these inserts are disclosed in SEQ ID NOS:363, 354, 356, 358, 360, 362, 359, 361, 357, 352, 355, and 353, respectively. When searched against Genbank, these prospective candidate sequences showed some degree of similarity to sequences disclosed in SEQ ID NOS:364-370. The Genbank search results are summarized in Table 2.

TABLE 2
Prospective candidate sequences show some similarity to
sequences in Genbank

| SEQ ID | Clone | Description of Genbank search hits | |
|---|---|---|---|
| SEARCHED | Identifier | showing some sequence similarity | |
| 355 | 63693 | FLJ21409 fis clone COL03924 | (SEQ ID NO: 364) |
| 361 | 63663 | Human eukaryotic initiation factor 4E | (SEQ ID NO: 365) |
| 359 | 63659 | KIAA0905 | (SEQ ID NO: 366) |
| 353 | 63949 | p35 CAK1-associated protein | (SEQ ID NO: 367) |
| 357 | 63677 | L6 Human Tumore Antigen | (SEQ ID NO: 368) |
| 358 | 62294 | ARF GTPase-activating protein GIT1 | (SEQ ID NO: 369) |
| 352 | 63676 | KIAA0104 | (SEQ ID NO: 370) |

### Example 4

### Identification and Analysis of Additional Colon Tumor Protein cDNAS

2,162 additional clones were subjected to DNA sequence analysis (disclosed herein as SEQ ID NOs:371-2,532). Of these, the cDNA clones depicted in SEQ ID NOs:371-1,452 were derived from PCR subtracted libraries CCLA1 and CCLS2. The cDNA clones depicted in SEQ ID NOs:1,453-2,532 were derived from the ADPS PCR-subtracted library.

To assess the utility of colon genes and/or antigens as immunological targets for immunothereapy in individuals with colon and/or metastatic colon cancer, and a potential diagnostic markers of colon and/or metastatic colon cancer, cDNA clones disclosed herein were further subjected to microarray expression analysis. Fifty three individual clones that showed two-fold overexpression in colon tumors comparing to normal tissues by microarray were identified by DNA sequencing and are disclosed herein as SEQ ID NOs:2,533-2,585. All 53 clones represented cDNA fragments from PCR-based subtracted cDNA libraries of colon tumors and metastatic colon tumors. Based on the combination of subraction library preparation and confirmed overexpression by microarray analysis, it was concluded that these 53 clones are over expressed in colon tumors and/or in metastatic colon tumors. These microarray expression data are presented herein as Table 3.

TABLE 3

| Element (384) | Element (96) | Ratio | Tumor Signal | Normal Signal |
|---|---|---|---|---|
| p0093r15c03 | R0359 E2 | 3.11 | 0.499 | 0.16 |
| p0091r16c23 | R0351 G12 | 3.5 | 0.148 | 0.042 |
| p0092r02c02 | R0352 D1 | 2.69 | 0.146 | 0.054 |
| p0092r13c22 | R0355 B11 | 2.02 | 0.348 | 0.172 |
| p0091r08c14 | R0349 H7 | 6.53 | 0.459 | 0.07 |
| p0093r10c12 | R0358 D6 | 2.63 | 0.265 | 0.101 |
| p0091r15c03 | R0351 E2 | 2.5 | 1.099 | 0.439 |
| p0092r04c21 | R0352 G11 | 2.88 | 0.461 | 0.16 |
| p0092r11c12 | R0354 F6 | 2.93 | 0.481 | 0.164 |
| p0091r07c13 | R0349 E7 | 2.3 | 0.108 | 0.047 |
| p0093r05c09 | R0357 A5 | 6.47 | 1.875 | 0.29 |
| p0093r06c23 | R0357 C12 | 6.65 | 1.387 | 0.209 |
| p0093r09c04 | R0358 B2 | 2.64 | 0.251 | 0.095 |
| p0092r06c04 | R0353 D2 | 2.29 | 1.42 | 0.619 |
| p0092r12c20 | R0354 H10 | 5.46 | 0.497 | 0.091 |
| p0091r06c08 | R0349 D4 | 2.68 | 0.677 | 0.252 |
| p0092r11c24 | R0354 F12 | 2.2 | 0.084 | 0.038 |
| p0093r16c19 | R0359 G10 | 3 | 0.229 | 0.076 |
| p0091r14c18 | R0351 D9 | 4.62 | 0.741 | 0.16 |
| p0092r10c19 | R0354 C10 | 2.39 | 0.045 | 0.019 |
| p0093r04c19 | R0356 G10 | 2.88 | 0.171 | 0.059 |
| p0092r09c03 | R0354 A2 | 2.51 | 0.34 | 0.136 |
| p0093r14c15 | R0359 C8 | 2.05 | 1.023 | 0.499 |
| p0093r06c11 | R0357 C6 | 2.29 | 0.332 | 0.145 |
| p0093r12c07 | R0358 G4 | 4.93 | 0.578 | 0.117 |
| p0092r15c24 | R0355 F12 | 2.22 | 0.061 | 0.027 |
| p0091r15c04 | R0351 F2 | 2.29 | 0.747 | 0.327 |
| p0091r13c20 | R0351 B10 | 2.1 | 0.051 | 0.024 |
| p0091r06c06 | R0349 D3 | 2.67 | 0.184 | 0.069 |
| p0093r05c23 | R0357 A12 | 2.95 | 0.199 | 0.067 |
| p0093r16c15 | R0359 G8 | 2.47 | 0.133 | 0.054 |
| p0092r10c20 | R0354 D10 | 2.78 | 0.128 | 0.046 |
| p0092r12c02 | R0354 H1 | 2.02 | 0.859 | 0.425 |
| p0091r12c19 | R0350 G10 | 8.76 | 0.792 | 0.09 |
| p0093r07c19 | R0357 E10 | 2.71 | 0.148 | 0.055 |
| p0093r12c11 | R0358 G6 | 2.58 | 0.244 | 0.094 |
| p0091r03c06 | R0348 F3 | 5.41 | 0.626 | 0.116 |
| p0092r14c11 | R0355 C6 | 2.39 | 0.141 | 0.059 |
| p0093r14c09 | R0359 C5 | 2.05 | 0.185 | 0.09 |
| p0091r03c12 | R0348 F6 | 2.29 | 0.164 | 0.072 |
| p0092r06c16 | R0353 D8 | 2.36 | 0.207 | 0.088 |
| p0093r01c16 | R0356 B8 | 2.39 | 0.388 | 0.162 |
| p0092r06c11 | R0353 C6 | 2.11 | 0.365 | 0.172 |
| p0091r05c14 | R0349 B7 | 6.47 | 0.861 | 0.133 |
| p0093r12c01 | R0358 G1 | 3.89 | 1.047 | 0.269 |
| p0091r05c08 | R0349 B4 | 4.86 | 0.359 | 0.074 |
| p0093r08c22 | R0357 H11 | 3.33 | 0.815 | 0.245 |
| p0092r16c19 | R0355 G10 | 2.7 | 2.19 | 0.812 |
| p0092r06c07 | R0353 C4 | 2.15 | 0.057 | 0.027 |
| p0093r16c20 | R0359 H10 | 2.16 | 0.233 | 0.108 |
| p0091r03c20 | R0348 F10 | 2.67 | 0.215 | 0.08 |
| p0091r10c20 | R0350 D10 | 2.84 | 0.161 | 0.056 |
| p0094r03c05 | R0360 E3 | 3.06 | 0.208 | 0.068 |

In subsequent analyses, additional clones were identified that showed at least two-fold overexpression by microarray in colon tumor cells as compared to colon normal cells. These clones are presented herein as Table 4.

TABLE 4

| Element (386) | Element (96) | Calculation | Ratio | Tumor Signal | Normal Signal |
|---|---|---|---|---|---|
| p0097r11c09 | R0374 E5 | Median | 2.07 | 0.165 | 0.08 |
| p0097r11c08 | R0374 F4 | Mean | 3.51 | 0.339 | 0.097 |
| p0097r12c20 | R0374 H10 | Mean | 2.83 | 0.334 | 0.118 |
| p0097r15c19 | R0375 E10 | Median | 2.7 | 0.211 | 0.078 |
| p0097r15c16 | R0375 F8 | Mean | 2.13 | 0.39 | 0.183 |
| p0097r16c14 | R0375 H7 | Mean | 2.01 | 0.139 | 0.069 |
| p0098r01c07 | R0376 A4 | Mean | 4 | 0.483 | 0.121 |
| p0098r01c22 | R0376 B11 | Median | 2.55 | 0.393 | 0.154 |
| p0098r01c24 | R0376 B12 | Mean | 2.05 | 0.298 | 0.145 |
| p0098r02c24 | R0376 D12 | Mean | 5.04 | 0.638 | 0.126 |
| p0098r03c10 | R0376 E5 | Median | 2.17 | 0.137 | 0.063 |
| p0098r04c03 | R0376 G2 | Median | 6.44 | 0.96 | 0.149 |
| p0098r06c17 | R0377 C9 | Median | 14.09 | 3.05 | 0.216 |
| p0098r06c24 | R0377 012 | Median | 2.1 | 0.241 | 0.115 |
| p0098r06c12 | R0377 06 | Median | 2.22 | 0.12 | 0.054 |
| p0098r07c22 | R0377 E11 | Mean | 2.49 | 0.17 | 0.068 |
| p0098r07c06 | R0377 E3 | Mean | 7.43 | 0.802 | 0.108 |
| p0098r08c20 | R0377 H10 | Mean | 2.77 | 0.15 | 0.054 |
| p0098r08c04 | R0377 H2 | Mean | 7.84 | 0.691 | 0.088 |
| p0098r08c16 | R0377 H8 | Median | 2.36 | 0.476 | 0.202 |
| p0098r09c07 | R0378 A4 | Mean | 2.12 | 0.369 | 0.174 |
| p0098r10c07 | R0378 C4 | Mean | 3.41 | 0.633 | 0.186 |
| p0098r10c04 | R0378 D2 | Mean | 5.57 | 0.656 | 0.118 |
| p0098r11c07 | R0378 E4 | Mean | 3.65 | 0.163 | 0.045 |
| p0098r11c10 | R0378 E5 | Mean | 2.29 | 0.437 | 0.191 |
| p0098r13c05 | R0379 A3 | Mean | 5.24 | 0.778 | 0.148 |
| p0098r13c06 | R0379 B3 | Mean | 2.21 | 0.297 | 0.135 |
| p0098r15c03 | R0379 E2 | Mean | 3.45 | 0.236 | 0.068 |
| p0098r15c24 | R0379 E12 | Median | 2.55 | 0.119 | 0.047 |
| p0098r16c22 | R0379 H11 | Median | 2.12 | 0.208 | 0.098 |
| p0099r01c19 | R0380 A10 | Median | 2.74 | 0.409 | 0.149 |
| p0099r01c22 | R0380 B11 | Median | 2.66 | 0.563 | 0.212 |
| p0099r02c03 | R0380 C2 | Median | 2.93 | 0.578 | 0.197 |
| p0099r02c07 | R0380 C4 | Mean | 2.84 | 0.297 | 0.105 |
| p0099r03c21 | R0380 E11 | Mean | 5.06 | 0.742 | 0.147 |
| p0099r03c09 | R0380 E5 | Mean | 2.05 | 0.338 | 0.165 |
| p0099r03c08 | R0380 E4 | Median | 6.45 | 1.31 | 0.203 |
| p0099r03c10 | R0380 E5 | Mean | 2.54 | 0.217 | 0.085 |
| p0099r03c14 | R0380 F7 | Mean | 2.28 | 0.11 | 0.048 |
| p0099r03c18 | R0380 F9 | Mean | 2.2 | 0.195 | 0.088 |
| p0099r05c23 | R0381 A12 | Mean | 2.04 | 0.248 | 0.122 |
| p0099r05c09 | R0381 A5 | Mean | 3.76 | 0.744 | 0.198 |
| p0099r05c20 | R0381 B10 | Mean | 4.42 | 0.115 | 0.026 |
| p0099r06c16 | R0381 D8 | Mean | 7.17 | 0.806 | 0.112 |
| p0099r07c02 | R0381 F1 | Mean | 2.52 | 0.14 | 0.056 |
| p0099r07c14 | R0381 F7 | Mean | 6.68 | 0.58 | 0.087 |
| p0099r08c19 | R0381 G10 | Median | 3 | 0.501 | 0.167 |
| p0099r08c22 | R0381 H11 | Mean | 4.99 | 0.419 | 0.084 |
| p0099r11c05 | R0382 E3 | Mean | 2.4 | 0.261 | 0.109 |
| p0099r11c24 | R0382 F12 | Mean | 2.49 | 0.232 | 0.093 |
| p0099r11c16 | R0382 F8 | Mean | 2.05 | 0.287 | 0.14 |
| p0099r12c11 | R0382 G6 | Mean | 2.22 | 0.29 | 0.131 |
| p0099r12c13 | R0382 G7 | Mean | 7.1 | 0.849 | 0.12 |
| p0099r13c16 | R0383 B8 | Median | 2.64 | 0.474 | 0.179 |
| p0099r15c21 | R0383 E11 | Mean | 2.11 | 0.317 | 0.15 |
| p0099r15c02 | R0383 F1 | Mean | 5.36 | 0.896 | 0.167 |
| p0099r16c05 | R0383 G3 | Mean | 2.75 | 0.415 | 0.151 |
| p0099r16c06 | R0383 H3 | Mean | 2.07 | 0.333 | 0.161 |
| p0100r01c17 | R0384 A9 | Median | 2.25 | 0.319 | 0.142 |
| p0100r01c04 | R0384 B2 | Mean | 6.98 | 1.341 | 0.192 |
| p0100r01c16 | R0384 B8 | Mean | 8.51 | 0.949 | 0.112 |
| p0100r03c02 | R0384 F1 | Median | 2.39 | 0.468 | 0.196 |
| p0100r06c07 | R0385 C4 | Mean | 3.21 | 0.407 | 0.127 |
| p0100r06c17 | R0385 C9 | Mean | 2.6 | 0.299 | 0.115 |
| p0100r06c12 | R0385 D6 | Mean | 2.48 | 0.271 | 0.109 |
| p0100r07c10 | R0385 E5 | Mean | 2.36 | 0.286 | 0.121 |
| p0100r10c12 | R0386 D6 | Mean | 2.47 | 0.199 | 0.081 |
| p0100r12c18 | R0386 H9 | Mean | 2.75 | 0.355 | 0.129 |
| p0100r13c20 | R0387 B10 | Mean | 2.25 | 0.266 | 0.118 |
| p0100r15c15 | R0387 E8 | Mean | 2.42 | 0.159 | 0.066 |
| p0100r15c17 | R0387 E9 | Median | 2.01 | 0.187 | 0.093 |
| p0101r01c22 | R0388 B11 | Mean | 2.09 | 0.238 | 0.114 |
| p0101r01c08 | R0388 B4 | Mean | 3.77 | 0.191 | 0.051 |
| p0101r03c08 | R0388 F4 | Median | 3.11 | 0.621 | 0.2 |
| p0101r04c11 | R0388 G6 | Mean | 2.06 | 0.119 | 0.058 |
| p0101r04c16 | R0388 H8 | Mean | 2.02 | 0.129 | 0.064 |
| p0101r05c07 | R0389 A4 | Mean | 2.05 | 0.228 | 0.111 |
| p0101r05c08 | R0389 B4 | Mean | 5.08 | 0.962 | 0.189 |
| p0101r06c05 | R0389 C3 | Median | 2.04 | 0.319 | 0.157 |
| p0101r06c22 | R0389 D11 | Mean | 2.21 | 0.307 | 0.139 |
| p0101r07c01 | R0389 E1 | Median | 3.02 | 0.339 | 0.112 |
| p0101r07c15 | R0389 E8 | Median | 2.54 | 0.213 | 0.084 |
| p0101r08c20 | R0389 H10 | Median | 2.3 | 0.202 | 0.088 |
| p0101r08c24 | R0389 H12 | Mean | 2.28 | 0.311 | 0.136 |
| p0101r09c24 | R0390 B12 | Mean | 4.93 | 0.635 | 0.129 |
| p0101r11c23 | R0390 E12 | Mean | 3.97 | 0.281 | 0.071 |
| p0101r11c14 | R0390 F7 | Median | 2.26 | 0.332 | 0.147 |
| p0101r11c16 | R0390 F8 | Mean | 3.77 | 0.259 | 0.069 |
| p0101r12c11 | R0390 G6 | Mean | 4.2 | 0.39 | 0.093 |
| p0101r13c02 | R0391 B1 | Mean | 5.03 | 0.742 | 0.148 |
| p0101r13c16 | R0391 B8 | Mean | 2.69 | 0.335 | 0.125 |
| p0101r15c06 | R0391 F3 | Median | 2.3 | 0.194 | 0.085 |
| p0102r01c17 | R0392 A9 | Mean | 2.03 | 0.286 | 0.141 |
| p0102r01c08 | R0392 B4 | Mean | 2.54 | 0.23 | 0.091 |
| p0102r01c12 | R0392 B6 | Mean | 2.11 | 0.342 | 0.162 |
| p0102r01c18 | R0392 B9 | Mean | 4.29 | 0.833 | 0.194 |
| p0102r02c19 | R0392 C10 | Median | 2.08 | 0.295 | 0.142 |
| p0102r03c13 | R0392 E7 | Mean | 3.67 | 0.33 | 0.09 |
| p0102r03c10 | R0392 F5 | Mean | 3.06 | 0.446 | 0.146 |
| p0102r04c01 | R0392 G1 | Median | 2.57 | 0.191 | 0.074 |
| p0102r04c11 | R0392 G6 | Median | 4.85 | 0.986 | 0.204 |
| p0102r05c08 | R0393 B4 | Mean | 2.41 | 0.361 | 0.149 |
| p0102r05c16 | R0393 B8 | Mean | 3.77 | 0.439 | 0.116 |
| p0102r06c09 | R0393 C5 | Mean | 2.05 | 0.37 | 0.181 |
| p0102r08c15 | R0393 G8 | Median | 2.05 | 0.149 | 0.073 |
| p0102r08c04 | R0393 H2 | Mean | 3.68 | 0.54 | 0.147 |
| p0102r08c08 | R0393 H4 | Mean | 3.4 | 0.378 | 0.111 |
| p0102r08c10 | R0393 H5 | Mean | 7.64 | 0.672 | 0.088 |
| p0102r08c12 | R0393 H6 | Mean | 2.56 | 0.357 | 0.139 |
| p0102r10c19 | R0394 C10 | Mean | 2.28 | 0.332 | 0.145 |
| p0102r10c04 | R0394 D2 | Median | 3.46 | 0.594 | 0.171 |
| p0102r10c08 | R0394 D4 | Mean | 5 | 0.715 | 0.143 |
| p0102r11c21 | R0394 E11 | Mean | 4.38 | 0.769 | 0.175 |
| p0102r11c11 | R0394 E6 | Median | 6.5 | 0.46 | 0.071 |
| p0102r11c14 | R0394 F7 | Mean | 6.42 | 0.948 | 0.148 |
| p0102r12c06 | R0394 H3 | Mean | 2.65 | 0.334 | 0.126 |
| p0102r13c12 | R0395 B6 | Median | 2.15 | 0.136 | 0.063 |
| p0102r14c09 | R0395 C5 | Mean | 2.64 | 0.519 | 0.196 |
| p0102r15c08 | R0395 F4 | Mean | 5.44 | 0.59 | 0.108 |
| p0102r15c18 | R0395 F9 | Median | 5.86 | 0.931 | 0.159 |
| p0102r16c24 | R0395 H12 | Mean | 7.7 | 0.821 | 0.107 |
| p0102r16c04 | R0395 H2 | Median | 2.11 | 0.155 | 0.073 |
| p0103r02c23 | R0396 C12 | Mean | 2.34 | 0.213 | 0.091 |
| p0103r02c03 | R0396 C2 | Mean | 3.09 | 0.541 | 0.175 |
| p0103r02c20 | R0396 D10 | Median | 2.4 | 0.214 | 0.089 |
| p0103r03c19 | R0396 E10 | Mean | 2.19 | 0.15 | 0.069 |
| p0103r03c23 | R0396 E12 | Mean | 3.44 | 0.595 | 0.173 |
| p0103r03c05 | R0396 E3 | Mean | 4.39 | 0.726 | 0.165 |
| p0103r04c22 | R0396 H11 | Mean | 5.37 | 0.832 | 0.155 |
| p0103r06c01 | R0397 C1 | Mean | 2.64 | 0.384 | 0.145 |
| p0103r08c17 | R0397 G9 | Mean | 3.57 | 0.57 | 0.16 |
| p0103r09c09 | R0398 A5 | Mean | 2.81 | 0.297 | 0.106 |
| p0103r09c06 | R0398 B3 | Mean | 4.28 | 0.791 | 0.185 |
| p0103r10c21 | R0398 C11 | Mean | 2.91 | 0.36 | 0.124 |
| p0103r11c09 | R0398 E5 | Mean | 10.75 | 1.524 | 0.142 |
| p0103r12c04 | R0398 H2 | Mean | 5.51 | 0.765 | 0.139 |
| p0103r14c23 | R0399 C12 | Mean | 2.8 | 0.295 | 0.105 |
| p0103r14c06 | R0399 D3 | Median | 2.07 | 0.275 | 0.133 |
| p0103r14c16 | R0399 D8 | Mean | 6.45 | 1.184 | 0.184 |
| p0103r15c21 | R0399 E11 | Mean | 3.01 | 0.6 | 0.2 |
| p0103r15c03 | R0399 E2 | Median | 2.1 | 0.197 | 0.094 |
| p0103r15c05 | R0399 E3 | Mean | 2.62 | 0.325 | 0.124 |
| p0103r15c12 | R0399 F6 | Mean | 2.24 | 0.296 | 0.132 |
| p0103r16c21 | R0399 G11 | Median | 2.12 | 0.264 | 0.124 |
| p0103r16c20 | R0399 H10 | Median | 6.23 | 1.42 | 0.228 |
| p0104r01c20 | R0400 B10 | Mean | 2.29 | 0.259 | 0.113 |
| p0104r01c12 | R0400 B6 | Mean | 3.29 | 0.461 | 0.14 |
| p0104r01c16 | R0400 B8 | Mean | 2.75 | 0.244 | 0.089 |
| p0104r02c14 | R0400 D7 | Mean | 2.03 | 0.41 | 0.201 |
| p0104r03c16 | R0400 F8 | Median | 2.03 | 0.246 | 0.121 |
| p0104r04c21 | R0400 G11 | Mean | 2.67 | 0.417 | 0.156 |
| p0104r04c16 | R0400 H8 | Mean | 8.29 | 1.275 | 0.154 |
| p0104r04c18 | R0400 H9 | Median | 2.05 | 0.208 | 0.102 |
| p0104r13c21 | R0403 A11 | Mean | 2.11 | 0.149 | 0.07 |
| p0104r13c05 | R0403 A3 | Median | 18.7 | 2.15 | 0.115 |
| p0104r13c07 | R0403 A4 | Mean | 3.15 | 0.585 | 0.186 |
| p0104r14c21 | R0403 C11 | Mean | 2.1 | 0.299 | 0.143 |
| p0104r15c05 | R0403 E3 | Median | 2.07 | 0.208 | 0.101 |
| p0104r15c06 | R0403 F3 | Mean | 2.57 | 0.421 | 0.164 |
| p0104r16c22 | R0403 H11 | Mean | 2.14 | 0.169 | 0.079 |
| p0104r16c24 | R0403 H12 | Mean | 2.54 | 0.503 | 0.198 |
| p0105r01c03 | R0404 A2 | Median | 10.31 | 0.687 | 0.067 |
| p0105r02c02 | R0404 D1 | Median | 3.45 | 0.303 | 0.088 |
| p0105r02c04 | R0404 D2 | Median | 2.57 | 0.112 | 0.044 |
| p0105r02c12 | R0404 D6 | Median | 2.33 | 0.1 | 0.043 |
| p0105r02c14 | R0404 D7 | Median | 2.41 | 0.269 | 0.112 |
| p0105r02c18 | R0404 D9 | Median | 2.96 | 0.287 | 0.097 |
| p0105r03c09 | R0404 E5 | Median | 2.45 | 0.319 | 0.13 |
| p0105r04c17 | R0404 G9 | Median | 4.67 | 0.296 | 0.063 |
| p0105r05c04 | R0405 B2 | Median | 2.37 | 0.258 | 0.109 |
| p0105r06c23 | R0405 C12 | Median | 2.01 | 0.114 | 0.057 |
| p0105r06c11 | R0405 C6 | Median | 7.24 | 0.194 | 0.027 |
| p0105r06c13 | R0405 C7 | Median | 2.37 | 0.158 | 0.067 |
| p0105r06c15 | R0405 C8 | Median | 7.17 | 0.21 | 0.029 |
| p0105r06c06 | R0405 D3 | Median | 2.09 | 0.262 | 0.126 |
| p0105r06c08 | R0405 04 | Median | 3.17 | 0.214 | 0.067 |
| p0105r07c22 | R0405 E11 | Median | 3.78 | 0.367 | 0.097 |

### Example 5

### Analysis of mRNA Expression Profiles of Colon Tumor Protein cDNAs Using Real-Time PCR

Four colon chip 4a clones, referred to as C1466P (SEQ ID NOs:854, 1346, 2586, and 2593), C1465P (SEQ ID NOs:1469, 1821, 149, 2515, 2417, 2587, and 2592), C1446P (SEQ ID NOs:188, 2183, 2415, 1554, 2588, and 2591), and C1444P (SEQ ID NOs:139, 142, 1433, 1788, 1810, 1651, 1875, 2589, and 2590) were further analyzed by real-time PCR. The first-strand cDNA used in the quantitative real-time PCR was synthesized from 20 μg of total RNA that was treated with DNase I (Amplification Grade, Gibco BRL Life Technology, Gaithersburg, Md.), using Superscript Reverse Transcriptase (RT) (Gibco BRL Life Technology, Gaithersburg, Md.). Real-time PCR was performed with a GeneAmp™ 5700 sequence detection system (PE Biosystems, Foster City, Calif.). The 5700 system uses SYBR™ green, a fluorescent dye that only intercalates into double stranded DNA, and a set of gene-specific forward and reverse primers. The increase in fluorescence was monitored during the whole amplification process. The optimal concentration of primers was determined using a checkerboard approach and a pool of cDNAs from breast tumors was used in this process. The PCR reaction was performed in 25 μl volumes that included 2.5 μl of SYBR green buffer, 2 μl of cDNA template and 2.5 μl each of the forward and reverse primers for the gene of interest. The cDNAs used for RT reactions were diluted 1:10 for each gene of interest and 1:100 for the β-actin control. In order to quantitate the amount of specific cDNA (and hence initial mRNA) in the sample, a standard curve was generated for each run using the plasmid DNA containing the gene of interest. Standard curves were generated using the Ct values determined in the real-time PCR which were related to the initial cDNA concentration used in the assay. Standard dilution ranging from 20-2×10⁶ copies of the gene of interest was used for this purpose. In addition, a standard curve was generated for β-actin ranging from 200 fg-2000 fg. This enabled standardization of the initial RNA content of a tissue sample to the amount of β-actin for comparison purposes. The mean copy number for each group of tissues tested was normalized to a constant amount of β-actin, allowing the evaluation of the over-expression levels seen with each of the genes.

Table 5 summarizes the real-time expression ratios and the 1-plate real time PCR data (colon tumors versus normal tissues) for the above-mentioned clones. Also included in Table 5 are the Genbank search results and the library of origin for each clone. Three PCR subtraction isolates were found for the C1444P candidate gene. The full length cDNA (SEQ ID NOs:2586-2589) and protein sequences (SEQ ID NOs:2590-2593) for each candidate are disclosed herein. Real-time PCR analysis shows that these candidate genes are overexpressed in most colon tumors when compared to normal colon and other normal tissues.

TABLE 5

| SEQ ID NO | | | | | | |
|---|---|---|---|---|---|---|
| FOR FL | ELEMENT | | | | REAL TIME RESULTS, 1 PLATE | CANDIDATE |
| cDNA/PRO: | (96) | RATIO | LIBRARY | GENBANK | PANEL* | NAME |
| 2589/2590 | R0391 E5 | 6.65 | ADPS | No match | CT overexpression, low/no | C1444P |
| | | | | | CN, high pancreas | |
| 2589/2590 | R0391 H1 | 2.83 | ADPS | Homo sapiens claudin-2 | CT overexpression, low/no | C1444P |
| | | | | mRNA, complete cds | CN, high pancreas, liver | |
| 2589/2590 | R0394 E10 | 3.56 | ADPS | Claudin 2 | CT overexpression, low/no | C1444P |
| | | | | | CN, high pancreas, liver | |
| 2587/2592 | R0392 A9 | 4.74 | ADPS | Human homeobox protein | Tissue specific, all CT and | C1465P |
| | | | | Cdx1 mRNA, complete cds | CN. Clean normals | C1465P |
| 2586/2593 | R0385 F5 | 9.07 | CCLS2 | Human DNA topoisomerase | Colon tumor overexpressed; | C1466P |
| | | | | II (top2) mRNA, complete | CN, esoph, low in all others | |
| | | | | cds | | |

*CT: colon tumor;
CN: colon normal;
esoph: esophagus.

### Example 6

### Identification and Analysis of Additional Colon Tumor Protein cDNAs

Further analysis of microarray data from the CTLS 1 subtraction library described in Example 1 revealed 8 additional clones that showed greater than 2-fold overexpression in colon tumors versus a set of normal tissues including normal colon. These cDNAs are disclosed in SEQ ID NOs:2594-2602. The sequences were searched against Genbank and the results are shown in Table 6. The probes used for the microarray analysis are listed in Table 7.

One clone, (element RO372 A11, C638S) was further analyzed using real-time PCR as described in Example 5. The real-time analysis showed that this gene is overexpressed in 75% of colon tumors but has low expression in normal colon tissue. Overexpression was also observed in skeletal muscle and adrenal gland (see Table 6). When searched against Genbank, C638S showed similarity to H. sapiens hiwi mRNA. The full-length cDNA and protein sequence for C638S are disclosed in SEQ ID NOs:2602 and 2603, respectively.

TABLE 6

| SEQ ID | | | | | | | REAL TIME PCR NORMAL |
|---|---|---|---|---|---|---|---|
| NO: | GENBANK IDENTITY | ELEMENT | RATIO | NAME | CT | CN | TISSUE EXPRESSION |
| 2600 | Homo sapiens hiwi mRNA | R0372 A11 | 2.13 | C638S | 75% | Low | Low levels in skeletal |
| | | | | | | | muscle and adrenal gland |
| 2601 | Homo sapiens cDNA: FLJ21212 | R0373 A2 | 2.11 | | | | |
| | fis, clone COL00502 | | | | | | |
| 2596 | Homo sapiens ribosomal protein | R0366 G6 | 2.03 | | | | |
| | S4, X-linked (RPS4X) mRNA | | | | | | |
| 2595 | Human carbohydrate | R0364 B8 | 2.32 | | | | |
| | sulfotransferase 4 | | | | | | |
| 2598 | Homo sapiens H2A histone | R0369 H4 | 2.01 | | | | |
| | family, member Z (H2AFZ) | | | | | | |
| | mRNA | | | | | | |
| 2594 | Homo sapiens hypothetical | R0363 E1 | 2.65 | | | | |
| | protein (HSPC236), mRNA | | | | | | |
| 2604 | Human proteasome (prosome, | R0362 E12 | 2.03 | | | | |
| | macropain) subunit, alpha type, 5 | | | | | | |
| 2599 | Homo sapiens S100 calcium- | R0370 B6 | 2.44 | | | | |
| | binding protein A6 (calcyclin) | | | | | | |
| 2597 | Serine protease inhibitor, Kunitz | R0366 B10 | 2.44 | | | | |
| | type, 2 | | | | | | |

TABLE 7

| PROBE | CY3 | | CY5 | CY5 |
|---|---|---|---|---|
| PAIR | PROBE | CY3 TISSUE | PROBE | TISSUE |
| 1 | 1016A | Colon Tumor, Duke's D | SPACT94 | Bone |
| | | | | Marrow |
| 2 | 1000A | Colon Tumor, Duke's A | 1003a | Normal |
| | | | | Colon |
| 3 | 647A | Colon Tumor, Duke's A | SPACT87 | Heart N |
| 4 | 645A | Colon Tumor, Duke's A | SPACT103 | Lung N |
| 5 | 1026A | Colon Tumor, Duke's B | 1065B | Aorta N |
| 6 | 633A | Colon Tumor, Duke's B | 119A | Kidney N |
| 7 | 235A | Colon Tumor, Duke's B | SPACT97 | Salivary |
| | | | | Gland N |
| 8 | 653A | Colon Tumor, Duke's C | 490/610C | Skin N |
| 9 | 1018A | Colon Tumor, Duke's C | SPACT85 | Brain N |
| 10 | 863A2 | Colon Tumor, Duke's C | SPACT89 | Pancreas N |
| 11 | 240A | Colon Tumor, Duke's C | SPACT93 | Trachea N |
| 12 | 659A | Colon Tumor, Duke's D | 1195A | PBMA |
| | | | | Resting |
| 13 | 672A | Colon Tumor, Duke's D | SPACT104 | Lymph |
| | | | | Node N |

### Example 7

### Identification and Analysis of an Additional Colon Tumor Antigen

Clone R0400 B8 originated from the ADPS PCR subtraction library, in which colon tumor was subtracted with normal colon as well as other tissues according to the usual protocol as described in Example 1. The ADPS subtraction was performed using Duke stage A and Duke stage D colon adenocarcinoma samples. This clone was identified as having greater than 2-fold overexpression in colon tumors as compared to normal tissue by microarray analysis. The sequence was searched against Genbank and showed some degree of similarity to a known gene referred to as small inducible cytokine subfamily A (SCYA20) or MIP3a. The full-length cDNA and protein sequences for this gene are disclosed in SEQ ID NOs:2605 and 2606, respectively.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed:

1. An isolated polynucleotide comprising a sequence selected from the group consisting of:
(a) sequences provided in SEQ ID NO:1-2589, 2594-2602, and 2604-2605;
(b) complements of the sequences provided in SEQ ID NO:1-2589, 2594-2602, and 2604-2605;
(c) sequences consisting of at least 20 contiguous residues of a sequence provided in SEQ ID NO:1-2589, 2594-2602, and 2604-2605;
(d) sequences that hybridize to a sequence provided in SEQ ID NO:1-2589, 2594-2602, and 2604-2605, under moderately stringent conditions;
(e) sequences having at least 75% identity to a sequence of SEQ ID NO:1-2589, 2594-2602, and 2604-2605;
(f) sequences having at least 90% identity to a sequence of SEQ ID NO:1-2589, 2594-2602, and 2604-2605; and
(g) degenerate variants of a sequence provided in SEQ ID NO:1-2589, 2594-2602, and 2604-2605.

2. An isolated polypeptide comprising an amino acid sequence selected from the group consisting of:
(a) sequences encoded by a polynucleotide of claim 1; and
(b) sequences having at least 70% identity to a sequence encoded by a polynucleotide of claim 1; and
(c) sequences having at least 90% identity to a sequence encoded by a polynucleotide of claim 1.
(d) sequences set forth in SEQ ID NO:2590-2593, 2603, and 2606;
(e) sequences having at least 70% identity to a sequence set forth in SEQ ID NO:2590-2593, 2603, and 2606; and
(f) sequences having at least 90% identity to a sequence set forth in SEQ ID NO:2590-2593, 2603, and 2606.

3. An expression vector comprising a polynucleotide of claim 1 operably linked to an expression control sequence.

4. A host cell transformed or transfected with an expression vector according to claim 3.

5. An isolated antibody, or antigen-binding fragment thereof, that specifically binds to a polypeptide of claim 2.

6. A method for detecting the presence of a cancer in a patient, comprising the steps of:
(a) obtaining a biological sample from the patient;
(b) contacting the biological sample with a binding agent that binds to a polypeptide of claim 2;
(c) detecting in the sample an amount of polypeptide that binds to the binding agent; and
(d) comparing the amount of polypeptide to a predetermined cut-off value and therefrom determining the presence of a cancer in the patient.

7. A fusion protein comprising at least one polypeptide according to claim 2.

8. An oligonucleotide that hybridizes to a sequence recited in SEQ ID NO:1-2589, 2594-2602, and 2604-2605 under moderately stringent conditions.

9. A method for stimulating and/or expanding T cells specific for a tumor protein, comprising contacting T cells with at least one component selected from the group consisting of:
(a) polypeptides according to claim 2;
(b) polynucleotides according to claim 1; and
(c) antigen-presenting cells that express a polynucleotide according to claim 1,
under conditions and for a time sufficient to permit the stimulation and/or expansion of T cells.

10. An isolated T cell population, comprising T cells prepared according to the method of claim 9.

11. A composition comprising a first component selected from the group consisting of physiologically acceptable carriers and immunostimulants, and a second component selected from the group consisting of:
(a) polypeptides according to claim 2;
(b) polynucleotides according to claim 1;
(c) antibodies according to claim 5;
(d) fusion proteins according to claim 7;
(e) T cell populations according to claim 10; and
(f) antigen presenting cells that express a polypeptide according to claim 2.

12. A method for stimulating an immune response in a patient, comprising administering to the patient a composition of claim 11.

13. A method for the treatment of a cancer in a patient, comprising administering to the patient a composition of claim 11.

14. A method for determining the presence of a cancer in a patient, comprising the steps of:
(a) obtaining a biological sample from the patient;
(b) contacting the biological sample with an oligonucleotide according to claim 8;
(c) detecting in the sample an amount of a polynucleotide that hybridizes to the oligonucleotide; and
(d) compare the amount of polynucleotide that hybridizes to the oligonucleotide to a predetermined cut-off value, and therefrom determining the presence of the cancer in the patient.

15. A diagnostic kit comprising at least one oligonucleotide according to claim 8.

16. A diagnostic kit comprising at least one antibody according to claim 5 and a detection reagent, wherein the detection reagent comprises a reporter group.

17. A method for inhibiting the development of a cancer in a patient, comprising the steps of:
(a) incubating CD4+ and/or CD8+ T cells isolated from a patient with at least one component selected from the group consisting of: (i) polypeptides according to claim 2; (ii) polynucleotides according to claim 1; and (iii) antigen presenting cells that express a polypeptide of claim 2, such that T cell proliferate;
(b) administering to the patient an effective amount of the proliferated T cells,
and thereby inhibiting the development of a cancer in the patient.
